SEQUENCE LISTING (267 sequences)

SEQ ID NO: 1 - DNA, 400 bp, Artificial sequence
  Oligo-5_GFP-siRNA1__hsa-mir150
ccaacctgtccctgccccttcctgccctctttgatgcggccccacttcctctggcaggaacccccgccctccctggacctgggtataaggcagggactgggcccacggggaggcagcgtccccgaggcagcagcggcagcggcggctcctctccccatggccctgtaagtcgtgctgcttcatgtggctgggctcagacaacatgaacaggccgacttacagggacctggggaccccggcaccggcaggccccaaggggtgaggtgagcgggcattgggacctcccctccctgtactcccatctctgctgcggcttttatgcgtctctccccttcgggtcccacatatcctctggtgcgctcctgcctcaccgcccccaccccatgcctgtcgtccccac

SEQ ID NO: 2 - DNA, 400 bp, Artificial sequence
  Oligo-6_GFP-siRNA6__hsa-mir150
ccaacctgtccctgccccttcctgccctctttgatgcggccccacttcctctggcaggaacccccgccctccctggacctgggtataaggcagggactgggcccacggggaggcagcgtccccgaggcagcagcggcagcggcggctcctctccccatggccctgtagttgtactccagcttgtgccctgggctcagagtcacaagcggaccacaactacagggacctggggaccccggcaccggcaggccccaaggggtgaggtgagcgggcattgggacctcccctccctgtactcccatctctgctgcggcttttatgcgtctctccccttcgggtcccacatatcctctggtgcgctcctgcctcaccgcccccaccccatgcctgtcgtccccac

SEQ ID NO: 3 - DNA, 400 bp, Artificial sequence
  Oligo-7_TP53-siRNA1__hsa-mir150
ccaacctgtccctgccccttcctgccctctttgatgcggccccacttcctctggcaggaacccccgccctccctggacctgggtataaggcagggactgggcccacggggaggcagcgtccccgaggcagcagcggcagcggcggctcctctccccatggccctgtagattctcttcctctgtgcgcctgggctcagagagcacagagaaccgaatctacagggacctggggaccccggcaccggcaggccccaaggggtgaggtgagcgggcattgggacctcccctccctgtactcccatctctgctgcggcttttatgcgtctctccccttcgggtcccacatatcctctggtgcgctcctgcctcaccgcccccaccccatgcctgtcgtccccac

SEQ ID NO: 4 - DNA, 400 bp, Artificial sequence
  Oligo-8_TP53-siRNA2__hsa-mir150
ccaacctgtccctgccccttcctgccctctttgatgcggccccacttcctctggcaggaacccccgccctccctggacctgggtataaggcagggactgggcccacggggaggcagcgtccccgaggcagcagcggcagcggcggctcctctccccatggccctgttattctccatccagtggtttcctgggctcagagcaaccactgatcaagaataacagggacctggggaccccggcaccggcaggccccaaggggtgaggtgagcgggcattgggacctcccctccctgtactcccatctctgctgcggcttttatgcgtctctccccttcgggtcccacatatcctctggtgcgctcctgcctcaccgcccccaccccatgcctgtcgtccccac

SEQ ID NO: 5 - DNA, 23 bp, Artificial sequence
  sgRNA seq for human miR-150
ccagcactggtacaagggttggg

SEQ ID NO: 6 - DNA, 23 bp, Artificial Sequence
  sgRNA seq for human miR-150
ccaacccttgtaccagtgctggg

SEQ ID NO: 7 - DNA, 2451 bp, homo sapiens
cgtgctttccacgacggtgacacgcttccctggattggccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgtccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttctttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccaaaaaaaaaaaaaaaaaaaa

SEQ ID NO: 8 - DNA, 869 bp, homo sapiens
tcacgtgacccgggcgcgctgcggccgcccgcgcggacccggcgagaggcggcggcgggagcggcggtgatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggggctgcccctggccgagtcactgaagcgactgatgtccctgtctccaggacggcctcctctcctactttgggacgcccacgtggcagaccgtgaccatctttgtggcgggagtgctcaccgcctcactcaccatctggaagaagatgggctgaggcccccagctgccttggactgtgtttttcctccataaattatggcatttttctgggaggggtggggattgggggacgtgggcatttttcttacttttgtaattattggggggtgtggggaagagtggtcttgagggggtaataaacctccttcgggacacaaaaaaaaaaa

SEQ ID NO: 9 - DNA, 1839 bp, homo sapiens
gaggcgattgcgattgggtgagacccagtaaggatggaaagtgtagaggagacaggaatccacggctttggaaaaaggaaggacaaaactcaccaaaccagagcagggcaggaagtaacaatgagaaactgaaaaagaaacggaatggaaagctatgagacaggatgaaatttggcatggggtctgcccaggcatgtccatgccaggtgcccagggctgcttccacgacgtgggtcccctgccagatttgtggccccagggagcgccatggcccgcgcacgccaggagggcagctccccggagcccgtagagggcctggcccgcgacggcccgcgccccttcccgctcggccgcctggtgccctcggcagtgtcctgcggcctctgcgagcccggcctggctgccgcccccgccgcccccaccctgctgcccgctgcctacctctgcgcccccaccgccccacccgccgtcaccgccgccctggggggttcccgctggcctgggggtccccgcagccggccccgaggcccgcgcccggacggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattaggtgcctgcacccgcccggtggacgtcagggactcggggggcaggcccctcccacctcctgacaccctggccagcgcgggggactttctctgcaccatgtagcatactggactcccagccctgcctgtcccgggggcgggccggggcagccactccagccccagcccagcctggggtgcactgacggagatgcggactcctgggtccctggccaagaagccaggagagggacggctgatggactcagcatcggaaggtggcggtgaccgagggggtggggactgagccgcccgcctctgccgcccaccaccatctcaggaaaggctgttgtgctggtgcccgttccagctgcaggggtgacactggggggggggggctctcctctcggtgctccttcactctgggcctggcctcaggcccctggtgcttccccccctcctcctgggagggggcccgtgaagagcaaatgagccaaacgtgaccactagcctcctggagccagagagtggggctcgtttgccggttgctccagcccggcgcccagccatcttccctgagccagccggcgggtggtgggcatgcctgcctcaccttcatcagggggtggccaggaggggcccagactgtgaatcctgtgctctgcccgtgaccgccccccgccccatcaatcccattgcataggtttagagagagcacgtgtgaccactggcattcatttggggggtgggagattttggctgaagccgccccagccttagtccccagggccaagcgctggggggaagacggggagtcagggagggggggaaatctcggaagagggaggagtctgggagtggggagggatggcccagcctgtaagatactgtatatgcgctgctgtagataccggaatgaattttctgtacatgtttggttaattttttttgtacatgatttttgtatgtttccttttcaataaaatcagattggaacagtggaaaaaaaaaaa

SEQ ID NO: 10 - DNA, 1954 bp, homo sapiens
actggacaaaagcgtggtctctggcgcggggatctcagagtttcccgggcactcaccgtgtgtagttggcatctccgcgcgtccggacacccgatcccagcatccctgcctgcaggactgttcgtgttcagctcgcgtcctgcagctgtccgaggtgctccagttggaggctgaggttcccgggctctgtagctgagtgggcggcggcaccggcggagatgcctgggaagaaggcgcgcaagaacgctcaaccgagccccgcgcgggctccagcagagctggaagtcgagtgtgctactcaactcaggagatttggagacaaactgaacttccggcagaaacttctgaatctgatatccaaactcttctgctcaggaacctgactgcatcaaaaacttgcatgaggggactccttcaaaagagttttctcaggaggtgcacgtttcatcaatttgaagaaagactgcattgtaattgagaggaatgtgaaggtgcattcatgggtgcccttggaaacggaagatggaatacatcaaagtgaatttctgttcaagttttcccagattatcattctttgggatgagagaacattataaaaccactttgtttattttaaagcaagaatggaagacccttgaaaataaagaagtaattattgacacatttcttttttacttagagaatcgttctagtgtttttgccgaagattaccgctggcctactgtgaagggagatgacctgtgattagactgggcggctggggagaaacagttcagtgcattgttgttgttgctgtttttggtgttttgcttttcagtgccaactcagcacattgtatatgattcggtttatacatattaccttgttataatgaaaaaactcattctgagaacactgaaatgttatactcagtgttgatttcttcggtcactacacaacgtaaaatcatttgtttcttttgactcaaattgtattgcttctgttcagatgatctttcattcaatgtgttcctgttgggcgttactagaaactatggaaaactggaaaataactttgaaaaaattggataaagtataggagggttacttggggccagtaaatcagtagactgaacattcaatataataaaagaacatggggattttgtataaccagggataataaaaagaaaaaagaagttaatttttaattgatgtttttgaaacttagtagaacaaatattcagaagtaacttgataagatatgaatgtttctaaagaagtttctaaaggttcggaaaatgctccttgtcacattagtgtgcatcctacaaaaagtgatctcttaatgtaaattaagaatattttcataattggaatatacttttcttaaaaaaaaggaacagttagttctcatctagaatgaaagttccatatatgcattggtgaatatatatgtatacacatacttacatacttatatgggtatctgtatagataatttgtattagagtattatatagcttcttagtagggtctcaagtaagtttcattttttttatctgggctatatacagtcctcaaataaataatgtcttgattttatttcagcaggaataattttatttattttgcctatttataattaaagtatttttctttagtttgaaaatgtgtattaaagttacatttttgagttacaagagtcttataactacttgaatttttagttaaaatgtcttaatgtaggttgtagtcactttagatggaaaattacctcacatctgttttcttcagtattacttaagattgtttatttagtggtagagagttttttttttcagcctagaggcagctattttaccatctggtatttatggtctaatttgtatttaaacatatgcacacatataaaagttgatactgtggcagtaaactattaaaagttttcactgttcaaaaaaaaaaaaaaaaaa

SEQ ID NO: 11 - DNA, 3951 bp, homo sapiens
atcaatggagccctccccaacccgggcgttccccagcgaggcttccttcccatcctcctgaccaccggggcttttcgtgagctcgtctctgatctcgcgcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaacaaaaaaaaaaaaaaa

SEQ ID NO: 12 - DNA, 1147 bp, Artificial sequence
  eGFP nucleic acid sequence
atgtctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggaggtagatttatgcatcctcttgtcatgagaagtcgaattgttcccattctgtgtgttgcagctacagatggagatacatagagatactcgtggattttgcttagtgttgagttttgttctggttgtgaactaaaagtttatacatttgcaggaaataaatagccttttgtttaaatcaaaaggtcttacctatgttagtgtgaagcattggatcccaaagaactccaaaatgcgatgaggcatatttaatcttgtctggactagtaacaggttgggatgaccacctgtgaagctccaacaggattgcctcctcacgcaatgtttgaggtctgatgttcaatagcttgttttgtttcactttgctttggactttcttttcgccaatgagctatgtttctgatggttttcactcttttggtgtgtagagaaccatcttcttcaaggacgacggcaactacaagacccgcgccgaggtgaagttcgagggcgacaccctggtgaaccgcatcgagctgaagggcatcgacttcaaggaggacggcaacacctggggcacaagctggagtacaactacaacagccacaacgtctatatcatggccgacaagcagaagaaggcatcaaggtgaacttcaagatccgccacaacatcgaggacggcagcgtgcagctcgccgaccactacagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 13 - RNA, 84 nt, homo sapiens
cuccccauggcccugucucccaacccuuguaccagugcugggcucagacccugguacaggccugggggacagggaccuggggac

SEQ ID NO: 14 - RNA, 110 nt, homo sapiens
acccggcagugccuccaggcgcagggcagccccugcccaccgcacacugcgcugccccagacccacugugcgugugacagcggcugaucugugccugggcagcgcgaccc

SEQ ID NO: 15 - RNA, 83 nt, homo sapiens
cggggugagguaguagguugugugguuucagggcagugauguugccccucggaagauaacuauacaaccuacugccuucccug

SEQ ID NO: 16 - RNA, 84 nt, homo sapiens
ccagucacguccccuuaucacuuuuccagcccagcuuugugacuguaaguguuggacggagaacugauaaggguaggugauuga

SEQ ID NO: 17 - RNA, 110 nt, homo sapiens
ggcuacagucuuucuucaugugacucguggacuucccuuugucauccuaugccugagaauauaugaaggaggcugggaaggcaaagggacguucaauugucaucacuggc

SEQ ID NO: 18 - RNA, 84 nt, homo sapiens
ggccaguguugagaggcggagacuugggcaauugcuggacgcugcccugggcauugcacuugucucggucugacagugccggcc

SEQ ID NO: 19 - RNA, 110 nt, homo sapiens
ggccagcugugaguguuucuuuggcagugucuuagcugguuguugugagcaauaguaaggaagcaaucagcaaguauacugcccuagaagugcugcacguuguggggccc

SEQ ID NO: 20 - RNA, 84 nt, homo sapiens
gugcucgguuuguaggcagugucauuagcugauuguacuguggugguuacaaucacuaacuccacugccaucaaaacaaggcac

SEQ ID NO: 21 - RNA, 23 nt, homo sapiens
aggcaguguaguuagcugauugc

SEQ ID NO: 22 - DNA, 576 bp, Artificial sequence
  CMV sequence promoter
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 23 - DNA, 225 bp, Artificial sequence
  BGH poly(A)signal termination sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 24 - DNA, 1147 bp, Artificial sequence; identical to SEQ ID NO: 12
  Synthetic construct clone eGFP-OsP5SM_E/R eGFP (eGFP) gene

SEQ ID NO: 25 - DNA, 4140 bp, Artificial sequence
  Cas9 human codon-optimized
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 26 - DNA, 1182 bp, Artificial sequence
  EF1a core promoter
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 27 - DNA, 257 bp, Artificial sequence
  pol III (U6) promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacg

SEQ ID NO: 28 - DNA, 711 bp, Artificial sequence
  mCherry ORF nucleic acid sequence
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggcccagtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 29 - DNA, 22 bp, Artificial sequence
  Single strand oligonucleotide
ttcgcttgcagagagaaatcac

SEQ ID NO: 30 - DNA, 21 bp, Artificial sequence
  Single strand oligonucleotide
aagctcaggagggatagcgcc

SEQ ID NO: 31 - DNA, 23 bp, Artificial sequence
  Single strand oligonucleotide
cttgcagagagaaatcacagtgg

SEQ ID NO: 32 - DNA, 23 bp, Artificial sequence
  Single strand oligonucleotide
gcttacacagagaatcacagagg

SEQ ID NO: 33 - DNA, 23 bp, Artificial sequence
  Single strand oligonucleotide
aagaatctgtaaagctcaggagg

SEQ ID NO: 34 - DNA, 23 bp, Artificial sequence
  Single strand oligonucleotide
ctatccatcctgagtttcattgg

SEQ ID NO: 35 - DNA, 21 bp, Artificial sequence
  Single strand oligonucleotide
aagtcgtgctgcttcatgtgg

SEQ ID NO: 36 - DNA, 22 bp, Artificial sequence
  Single strand oligonucleotide
ccacataagcaggacgagttaa

SEQ ID NO: 37 - DNA, 21 bp, Artificial sequence
  Single strand oligonucleotide
agttgtactccagcttgtgcc

SEQ ID NO: 38 - DNA, 21 bp, Artificial sequence
  Single strand oligonucleotide
gcaaagctgcagtacaactaa

SEQ ID NO: 39 - DNA, 21 bp, Artificial sequence
  Single strand oligonucleotide
tatccacacaaactacctgca

SEQ ID NO: 40 - DNA, 21 bp, Artificial sequence
  Single strand oligonucleotide
gcagtagttagtgtggataaa

SEQ ID NO: 41 - DNA, 21 bp, Artificial sequence
  Single strand oligonucleotide
tgacaatccagccaatccagc

SEQ ID NO: 42 - DNA, 21 bp, Artificial sequence
  Single strand oligonucleotide
ctgattggcaggattgtcaaa

SEQ ID NO: 43 - DNA, 21 bp, Artificial sequence
  Single strand oligonucleotide
taaagatcggcaacacatgat

SEQ ID NO: 44 - DNA, 19 bp, Artificial sequence
  Single strand oligonucleotide
tcagtgttggcgatcttta

SEQ ID NO: 45 - DNA, 21 bp, Artificial sequence
  Single strand oligonucleotide
tgacctttcttgggtttagcc

SEQ ID NO: 46 - DNA, 19 bp, Artificial sequence
  Single strand oligonucleotide
gctaacccatgaaaggtca

SEQ ID NO: 47 - DNA, 102 bp, Artificial sequence
  Single strand oligonucleotide
taagtactttcgcttgcagagagaaatcacagtggtcaaaaaagttgtagttttcttaaagtctctttcctctgtgattctctgtgtaagcgaaagagcttg

SEQ ID NO: 48 - DNA, 102 bp, Artificial sequence
  Single strand oligonucleotide
taagtacttaagtcgtgctgcttcatgtggagtggtcaaaaaagttgtagttttcttaaagtctctttcctctccacataagcaggacgagttaagagcttg

SEQ ID NO: 49 - DNA, 102 bp, Artificial sequence
  Single strand oligonucleotide
taagtacttagttgtactccagcttgtgccagtggtcaaaaaagttgtagttttcttaaagtctctttcctctggcaaagctgcagtacaactaagagcttg

SEQ ID NO: 50 - DNA, 102 bp, Artificial sequence
  Single strand oligonucleotide
taagtactttatccacacaaactacctgcaagtggtcaaaaaagttgtagttttcttaaagtctctttcctcttgcagtagttagtgtggataaagagcttg

SEQ ID NO: 51 - DNA, 102 bp, Artificial sequence
  Single strand oligonucleotide
taagtactttgacaatccagccaatccagcagtggtcaaaaaagttgtagttttcttaaagtctctttcctctgctgattggcaggattgtcaaagagcttg

SEQ ID NO: 52 - DNA, 102 bp, Artificial sequence
  Single strand oligonucleotide
taagtactttaaagatcggcaacacatgatagtggtcaaaaaagttgtagttttcttaaagtctctttcctctgatcagtgttggcgatctttaagagcttg

SEQ ID NO: 53 - DNA, 102 bp, Artificial sequence
  Single strand oligonucleotide
taagtactttgacctttcttgggtttagccagtggtcaaaaaagttgtagttttcttaaagtctctttcctctgggctaacccatgaaaggtcaagagcttg

SEQ ID NO: 54 - DNA, 107 bp, Artificial sequence
  Single strand oligonucleotide
gtagagaagaatctgtaaagctcaggagggatagcgccatgatgatcacattcgttatctattttttggcgctatccatcctgagtttcattggctcttcttactac

SEQ ID NO: 55 - DNA, 107 bp, Artificial sequence
  Single strand oligonucleotide
gtagagaagaatctgtaaagtcgtgctgcttcatgtggatgatgatcacattcgttatctattttttccacatgaagaagcacgacttgattggctcttcttactac

SEQ ID NO: 56 - DNA, 107 bp, Artificial sequence
  Single strand oligonucleotide
gtagagaagaatctgtaagttgtactccagcttgtgccatgatgatcacattcgttatctattttttggcacaagcttgagtacaactgattggctcttcttactac

SEQ ID NO: 57 - DNA, 107 bp, Artificial sequence
  Single strand oligonucleotide
gtagagaagaatctgtatatccacacaaactacctgcaatgatgatcacattcgttatctatttttttgcaggtagtgtgtgtggatagattggctcttcttactac

SEQ ID NO: 58 - DNA, 107 bp, Artificial sequence
  Single strand oligonucleotide
gtagagaagaatctgtatgacaatccagccaatccagcatgatgatcacattcgttatctattttttgctggattggatggattgtcagattggctcttcttactac

SEQ ID NO: 59 - DNA, 107 bp, Artificial sequence
  Single strand oligonucleotide
gtagagaagaatctgtataaagatcggcaacacatgatatgatgatcacattcgttatctattttttatcatgtgttaccgatctttacattggctcttcttactac

SEQ ID NO: 60 - DNA, 107 bp, Artificial sequence
  Single strand oligonucleotide
gtagagaagaatctgtatgacctttcttgggtttagccatgatgatcacattcgttatctattttttggctaaaccccagaaaggtcacattggctcttcttactac

SEQ ID NO: 61 - DNA, 4100 bp, Artificial sequence
  Single strand oligonucleotide
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttcgcttgcagagagaaatcacagtggtcaaaaaagttgtagttttcttaaagtctctttcctctgtgattctctgtgtaagcgaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 62 - DNA, 4100 bp, Artificial sequence
  Single strand oligonucleotide
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtacttaagtcgtgctgcttcatgtggagtggtcaaaaaagttgtagttttcttaaagtctctttcctctccacataagcaggacgagttaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 63 - DNA, 4100 bp, Artificial sequence
  Single strand oligonucleotide
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtacttagttgtactccagcttgtgccagtggtcaaaaaagttgtagttttcttaaagtctctttcctctggcaaagctgcagtacaactaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 64 - DNA, 4100 bp, Artificial sequence
  Single strand oligonucleotide
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttatccacacaaactacctgcaagtggtcaaaaaagttgtagttttcttaaagtctctttcctcttgcagtagttagtgtggataaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 65 - DNA, 4100 bp, Artificial sequence
  Single strand oligonucleotide
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttgacaatccagccaatccagcagtggtcaaaaaagttgtagttttcttaaagtctctttcctctgctgattggcaggattgtcaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 66 - DNA, 4100 bp, Artificial sequence
  Single strand oligonucleotide
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttaaagatcggcaacacatgatagtggtcaaaaaagttgtagttttcttaaagtctctttcctctgatcagtgttggcgatctttaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 67 - DNA, 4100 bp, Artificial sequence
  Single strand oligonucleotide
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttgacctttcttgggtttagccagtggtcaaaaaagttgtagttttcttaaagtctctttcctctgggctaacccatgaaaggtcaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 68 - DNA, 4000 bp, Artificial sequence
  Single strand oligonucleotide
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaaagctcaggagggatagcgccatgatgatcacattcgttatctattttttggcgctatccatcctgagtttcattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 69 - DNA, 4000 bp, Artificial sequence
  Single strand oligonucleotide
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaaagtcgtgctgcttcatgtggatgatgatcacattcgttatctattttttccacatgaagaagcacgacttgattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 70 - DNA, 4000 bp, Artificial sequence
  Single strand oligonucleotide
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaagttgtactccagcttgtgccatgatgatcacattcgttatctattttttggcacaagcttgagtacaactgattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 71 - DNA, 4000 bp, Artificial sequence
  Single strand oligonucleotide
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatatccacacaaactacctgcaatgatgatcacattcgttatctatttttttgcaggtagtgtgtgtggatagattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 72 - DNA, 4000 bp, Artificial sequence
  Single strand oligonucleotide
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatgacaatccagccaatccagcatgatgatcacattcgttatctattttttgctggattggatggattgtcagattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 73 - DNA, 4000 bp, Artificial sequence
  Single strand oligonucleotide
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtataaagatcggcaacacatgatatgatgatcacattcgttatctattttttatcatgtgttaccgatctttacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 74 - DNA, 4000 bp, Artificial sequence
  Single strand oligonucleotide
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatgacctttcttgggtttagccatgatgatcacattcgttatctattttttggctaaaccccagaaaggtcacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 75 - DNA, 19 bp, Artificial sequence
  Single strand oligonucleotide
acaccctgggaattggttt

SEQ ID NO: 76 - DNA, 20 bp, Artificial sequence
  Single strand oligonucleotide
gtatgcgccaataagaccac

SEQ ID NO: 77 - DNA, 20 bp, Artificial sequence
  Single strand oligonucleotide
gtactgctggtcctttgcag

SEQ ID NO: 78 - DNA, 20 bp, Artificial sequence
  Single strand oligonucleotide
aggagcactacggaaggatg

SEQ ID NO: 79 - DNA, 22 bp, Artificial sequence
  Single strand oligonucleotide
gttgagagtgttggagaaggag

SEQ ID NO: 80 - DNA, 21 bp, Artificial sequence
  Single strand oligonucleotide
ctcggtgttgatcctgagaag

SEQ ID NO: 81 - DNA, 23 bp, Artificial sequence
  Single strand oligonucleotide
agcttccttcatttctgtgaatc

SEQ ID NO: 82 - DNA, 21 bp, Artificial sequence
  Single strand oligonucleotide
ctgttccccacttaccctgac

SEQ ID NO: 83 - DNA, 21 bp, Artificial sequence
  Single strand oligonucleotide
ctcctcatctgattccttctc

SEQ ID NO: 84 - DNA, 21 bp, Artificial sequence
  Single strand oligonucleotide
ctgttccccacttaccctgac

SEQ ID NO: 85 - DNA, 23 bp, Artificial sequence
  Single strand oligonucleotide
gtgtgtggaaagtttatcaacac

SEQ ID NO: 86 - DNA, 27 bp, Artificial sequence
  Single strand oligonucleotide
gttagtatgtaacatctccgattctac

SEQ ID NO: 87 - DNA, 20 bp, Artificial sequence
  Single strand oligonucleotide
agagtggtcgtgtgagttgc

SEQ ID NO: 88 - DNA, 25 bp, Artificial sequence
  Single strand oligonucleotide
ccagtcaagatttcatggatctgtt

SEQ ID NO: 89 - DNA, 28 bp, Artificial sequence
  Single strand oligonucleotide
ctaacataatcgagaacagatggaagac

SEQ ID NO: 90 - DNA, 32 bp, Artificial sequence
  Single strand oligonucleotide
ttctcatgtccttgatttatcaatttaacaac

SEQ ID NO: 91 - DNA, 20 bp, Artificial sequence
  Single strand oligonucleotide
agagtggtcgtgtgagttgc

SEQ ID NO: 92 - DNA, 26 bp, Artificial sequence
  Single strand oligonucleotide
cgcatcagatctatcaaacacataga

SEQ ID NO: 93 - DNA, 110 bp, Artificial sequence
  Single strand DNA oligonucleotide
gccgcaggaucuagggguuacucucuaggggguaugguauagcuuguaaccagccgccagaaaacuguccgcaaguuuaugcuguaucucacagacagcaaccgacuacg

SEQ ID NO: 94 - DNA, 63 bp, Artificial sequence
  Single strand DNA oligonucleotide
gucagccugaaccugcugcugaaaaaccucuaaauagggacccuccugguggguuagcucggc

SEQ ID NO: 95 - DNA, 110 bp, Artificial sequence
  Single strand DNA oligonucleotide
gccgccggaagguuugggaucccccuaugguggacuagggugguuuuacugcguucuagguaaucguuaaaaaguuuguuuuaguuuaucccaggagguuucugugaccg

SEQ ID NO: 96 - DNA, 95 bp, Artificial sequence
  Single strand DNA oligonucleotide
aggccucguagauuggauggccguaguggacugugaggguccccagaaaggucggauucgugggguucacuaauccgagauguagccgccucacc

SEQ ID NO: 97 - DNA, 110 bp, Artificial sequence
  Single strand DNA oligonucleotide
gcggcgggaaguauauggcacccccuaggguauucauggucgguuggaucgcggccgacgcaaacgucugccaacuugaucaugaauaucgcagguggcgucacgguaaa

SEQ ID NO: 98 - DNA, 63 bp, Artificial sequence
  Single strand DNA oligonucleotide
gucaggaacaggauauaauggauaaaccucaaaaaagggaccuuaccuguguccuaccucggc

SEQ ID NO: 99 - DNA, 110 bp, Artificial sequence
  Single strand DNA oligonucleotide
gccgcaggaaaggugaggaucccucaacggguguuuggggauuuuuuaggcugacacgggaaaccagacagagaagauuuccagauguucucugacggcuccuacgccgg

SEQ ID NO: 100 - DNA, 110 bp, Artificial sequence
  Single strand DNA oligonucleotide
ccaacacccuggcccagugcaauugauuggugcacuucgcggaggugugccggagcguuugagcauucgugggguucacuaauccgauggcagccggggaacacgauccu

SEQ ID NO: 101 - DNA, 110 bp, Artificial sequence
  Single strand DNA oligonucleotide
gcggccggaaaucccuuuaagccccuacguuauuuaugauauugcauaaccugacaugaguaaacagcaaauguauauauuguaaauaacacagguguaugagacggaca

SEQ ID NO: 102 - DNA, 79 bp, Artificial sequence
  Single strand DNA oligonucleotide
cguacaggaaaaugacucauuaauugaaaauaaacgugacuaagucuuauuuucauuuaugugggccaagguucuguau

SEQ ID NO: 103 - DNA, 110 bp, Artificial sequence
  Single strand DNA oligonucleotide
gccgcuggaaggcguagguacccucaaagggaguuuucgauguuuguagccugacugagggaaacagccaacaaauuguuggagauuuucccugauggcaucuuccgccg

SEQ ID NO: 104 - DNA, 81 bp, Artificial sequence
  Single strand DNA oligonucleotide
ugcgaugucgcaaggguuggugcucuugguggguuguaagagauaacauucgugggguucacuaaucccugcgacaucggg

SEQ ID NO: 105 - DNA, 110 bp, Artificial sequence
  Single strand DNA oligonucleotide
gcggcgggaauuggagggagccguccagguucuuuguugcggggugcacaccgaccgaacgaaacgggcagcaccuucgugguaaagaacacggagggguuccgcccaca

SEQ ID NO: 106 - DNA, 63 bp, Artificial sequence
  Single strand DNA oligonucleotide
guccgaucaaugcuccugaagauaaaccucuaaauagggacccuccggggggcaucucgcggc

SEQ ID NO: 107 - DNA, 86 bp, Artificial sequence
  Single strand DNA oligonucleotide
ugcuugauuccgcgacgcuuccuauuucaaaaaaaagcgggggaccaggggcuuuuucguggcagggcggugcccaucccugagcc

SEQ ID NO: 108 - DNA, 95 bp, Artificial sequence
  Single strand DNA oligonucleotide
aguccagcaccagaugggggcccuuggugagcguuccgggugggauaaaccggcauucgugggguucacuaauccauccugugagacgccgcaac

SEQ ID NO: 109 - DNA, 110 bp, Artificial sequence
  Single strand DNA oligonucleotide
gcggcgggaaggcggccccacccccuaggguauucuuggucggguugaaccgaguuucuggaugucgcuacaaccaugauuaagaauaucacaggcggcggggaaugcgg

SEQ ID NO: 110 - DNA, 73 bp, Artificial sequence
  Single strand DNA oligonucleotide
ugacgggagauuuuuauuuguggguagcgcugugacacuucaaacucuguuuggaaauaaaaauuaacuguca

SEQ ID NO: 111 - DNA, 86 bp, Artificial sequence
  Single strand DNA oligonucleotide
cgccacauuccgguauugaguauuuauucagguuaagaggaugcccugggucuuagucgaugcguuccguuccgcaugcccuggcc

SEQ ID NO: 112 - DNA, 94 bp, Artificial sequence
  Single strand DNA oligonucleotide
ccacgaaacggcuacgacgggggguuggagggguaaugccauugcggaguacugcguccuauaauggcauaaucacuccgcccccccggcgagg

SEQ ID NO: 113 - DNA, 110 bp, Artificial sequence
  Single strand DNA oligonucleotide
gccgccggaacucacagcuacacccgaggguguuggaugagcucgugaaaccggagcgucgaagcgggcacgugacgcuuauucaauaucgccgguugcagcuauugacc

SEQ ID NO: 114 - DNA, 85 bp, Artificial sequence
  Single strand DNA oligonucleotide
ugcccgggacccagccagaggaauccuuuacagauugugugcaguucagucuguaggccauuucuuugcucgggggccggugccc

SEQ ID NO: 115 - DNA, 86 bp, Artificial sequence
  Single strand DNA oligonucleotide
cgccguacgucgcguccagaggaauccuuuacagauugggacguccaaggcgaucuguugggcuguauggagccagugagccggcc

SEQ ID NO: 116 - DNA, 95 bp, Artificial sequence
  Single strand DNA oligonucleotide
gguggaacugcgccggggcgcauuuggugaguguuuugggucccuaaacggccgauucgugggguucacuaaucccggaggugauacagcgcauc

SEQ ID NO: 117 - DNA, 119 bp, Artificial sequence
  Single strand DNA oligonucleotide
aggguagcccaagggccggguauucuucuucguuggugauggaccgggguagagagacaaggccccgaguagaagaguccuucaucaacgaggaagagauucggcccuguggggaccca

SEQ ID NO: 118 - DNA, 71 bp, Artificial sequence
  Single strand DNA oligonucleotide
gcgacugaaagcuucaucaacgaggagcugugaagccacagaugggcuuuucguuugaaguuuucagcugc

SEQ ID NO: 119 - DNA, 80 bp, Artificial sequence
  Single strand DNA oligonucleotide
ccugcucccacaauucaucaacgaggaagagauuggaguaggcaggauuuauuuugcguuggagggaugugccuucuagg

SEQ ID NO: 120 - DNA, 119 bp, Artificial sequence
  Single strand DNA oligonucleotide
aggugcccgucgucuccggggauucgugggguucacuaauccauugggggagggaaaauaguccccaacaagaagaguggcuuagugagcuccacgggccccggagauggacgucgcca

SEQ ID NO: 121 - DNA, 119 bp, Artificial sequence
  Single strand DNA oligonucleotide
agggagcggcaguaugcagggcaaggcgaggcagcuugaguuaucgggggagagacaacaguccccguucagaagaauaaguuaggcugucucgccuucccugcguacgugcuaucuca

SEQ ID NO: 122 - DNA, 73 bp, Artificial sequence
  Single strand DNA oligonucleotide
cucugccucccgccccugaugagaucgaguacauuugguaugugcacagggucucauuucagcaggaacaggg

SEQ ID NO: 123 - DNA, 80 bp, Artificial sequence
  Single strand DNA oligonucleotide
ccuuaugccacaaccaggaaagacugauacagaaguauaagcccguucugucgugugcaaucauggaugugacugggagg

SEQ ID NO: 124 - DNA, 119 bp, Artificial sequence
  Single strand DNA oligonucleotide
aauucacagcccgucccugggguuggugggcuucaugaggggagcggggcagggagaaaaggccccgagaagaagacuucauucgugggguucacuaauccaggggcgaggcucggaua

SEQ ID NO: 125 - DNA, 119 bp, Artificial sequence
  Single strand DNA oligonucleotide
aggaugcugcgucuuggcguucguugggguuugggcagucggcguggggcagagacaacaggccccaaggagaagucgcccacugcuuaagccucaauaacgucgggagcgcaaaucca

SEQ ID NO: 126 - DNA, 77 bp, Artificial sequence
  Single strand DNA oligonucleotide
ccgggacccaguaggagaaauuuauaaaagauugugugcuguucagucuuuuguccauuucuucgcucggggaccgg

SEQ ID NO: 127 - DNA, 80 bp, Artificial sequence
  Single strand DNA oligonucleotide
ccugguaccacagaaagaaaaaauauaaauuaagguaccaguccgcuuaaucggucuuuguuauguaugugucggauagg

SEQ ID NO: 128 - DNA, 87 bp, Artificial sequence
  Single strand DNA oligonucleotide
cucagccgguaggucuuggagggguaaugccauugcguagagggacaagagauguuggcaucugucuccucuaagacuuacucugag

SEQ ID NO: 129 - DNA, 119 bp, Artificial sequence
  Single strand DNA oligonucleotide
aguuagccgcgggagccugggcaagcagaacauccaacugauaggggggcagggaaagaaggcccccaggagaagauuauaaguuggauguucuguuucccaggcucugcgcgauaaca

SEQ ID NO: 130 - DNA, 71 bp, Artificial sequence
  Single strand DNA oligonucleotide
guagcacugaggugcuuaugacauaauaaguguugagcuaucuuuuguguuaugagcacuuaaaguacugc

SEQ ID NO: 131 - DNA, 84 bp, Artificial sequence
  Single strand DNA oligonucleotide
gcacccgggcacuguuuuuuuuuuuuuuuuuucagugcuagacccucggggacaaacgagaaagaguaagucggcccuuggggc

SEQ ID NO: 132 - DNA, 119 bp, Artificial sequence
  Single strand DNA oligonucleotide
aguuuaccgcggggcccugggauucgugggguucacuaaucccgagggggagagauacgagcccccuaccagaagaugggcuuagugaacuccacgagcccgggguccgcgcgaagaca

SEQ ID NO: 133 - DNA, 119 bp, Artificial sequence
  Single strand DNA oligonucleotide
agggaucagcaugauccugggaucguugacaaugcaaaauuugugggggcaaagaaaccaggccccucagauaagaacagcuuuuguguugucagcggcccggggucagugcucuccca

SEQ ID NO: 134 - DNA, 71 bp, Artificial sequence
  Single strand DNA oligonucleotide
guagcaccaaagugcaaauggaaaauauaguguuuaguuaucuaguuuuucauuugcacuuaagguacugc

SEQ ID NO: 135 - DNA, 80 bp, Artificial sequence
  Single strand DNA oligonucleotide
cccgcucccacucuaccauaauaauaauaauaagggagcagggggcuuauucuugguguuguaguaaagugccacucggg

SEQ ID NO: 136 - DNA, 119 bp, Artificial sequence
  Single strand DNA oligonucleotide
aguaugccgcgugguccggggauuggagggguaaugccauugcgugguucagcgauaauagggaccaacgagaagccguacuggcauuacucuucugaccccggaccagcgcgaauaca

SEQ ID NO: 137 - DNA, 114 bp, Artificial sequence
  Single strand DNA oligonucleotide
gaaccgaguacugcaaccuuugggcgcuucauuauuccguuguggcggagaaaccgagcuguauugaaauaaugaauaaaggcaagcaacugagucagacguugcugaauggaa

SEQ ID NO: 138 - DNA, 88 bp, Artificial sequence
  Single strand DNA oligonucleotide
cgcgcgccucucaauaccuaaaauauuaauaguuaauguuuaccgauuaaaucuagcuguuagucuuuugguagcugcgagucgcgcc

SEQ ID NO: 139 - DNA, 125 bp, Artificial sequence
  Single strand DNA oligonucleotide
ccccagccacguaccuaggaagcccgacgauugacucggugaucuacauuuuuauauaagugaaugaugagcgggguguuuacuuugacgggacugcgccccacaguuaauaacacaaacucuag

SEQ ID NO: 140 - DNA, 126 bp, Artificial sequence
  Single strand DNA oligonucleotide
ccacccgaagaagaggaucagucacacgagggguccugcuguguuggagggguaaugccauugcacacacgcacugguaaugagguugucucuccccaugcagcggacccuaaaaccaccgcaaag

SEQ ID NO: 141 - DNA, 126 bp, Artificial sequence
  Single strand DNA oligonucleotide
ccccccggagccaaggaaguaccaaccaguggguguaggacacuccaaaaucaauccuaauuuacgacacaaacucugaauuccgguugguuuugacgguguccacguccaaacacgacagcgaag

SEQ ID NO: 142 - DNA, 107 bp, Artificial sequence
  Single strand DNA oligonucleotide
ggugucagcaccguucagggugauucagugucugugccgaggggcaagcacgccguucacccuuggcuauggcucuugaggaacccucagcaccaaccgcaccugcc

SEQ ID NO: 143 - DNA, 123 bp, Artificial sequence
  Single strand DNA oligonucleotide
gcggagcggcagaagguaccuaaagguggacauucagaaauaaggugcccgguucuuugucgcucuaaaugugauuaaacauggucggugcccuuguuuuucagugugagaacuagccacgaa

SEQ ID NO: 144 - DNA, 126 bp, Artificial sequence
  Single strand DNA oligonucleotide
cgacgcccagccccgcccccgaccagagcauggucuagaugagccuggcauuguucagucagcgcggcacagaauccguuggagggguaaugccauugcucaucaggccauaagaccacccccaag

SEQ ID NO: 145 - DNA, 126 bp, Artificial sequence
  Single strand DNA oligonucleotide
cacaccaaggaaaaggcccgauaucaagcugggaccgcugucgcagauuauacuugaucuaaaaagugaccgaugcuuuuggcacaagugugaucacgcgacaggguuccaaacaccaccucuaag

SEQ ID NO: 146 - DNA, 107 bp, Artificial sequence
  Single strand DNA oligonucleotide
ggugucagcaccgcucagggaguuugauugguggagcggaggggcaagcacuccguucacccuucgcauaauuucaucaauaucccucagcaccaaccgcaccugcc

SEQ ID NO: 147 - DNA, 125 bp, Artificial sequence
  Single strand DNA oligonucleotide
ccccagccccaguccccggcauuccugagugguacucggagagucugggaguugaauuuaaaaauuguaaccagggcguuuuagcucccggcugucaaagccucaguaccaaaaccgccuccggg

SEQ ID NO: 148 - DNA, 126 bp, Artificial sequence
  Single strand DNA oligonucleotide
cacaccacgaccccggcccgaccauacgcuggaucuguaaucguuuggcauugucuagcugacgcaagacauaguucguuggagggguaaugccauugcgauuaagauccaaaaucgaccacaaag

SEQ ID NO: 149 - DNA, 126 bp, Artificial sequence
  Single strand DNA oligonucleotide
cccagcgccgacaagcccaaaccagaacggggauccgcccguuuugcaggauccggauccguauaguaacgccagcguacggugucggguccugcucgggcgggggauccuaauacuaccucgaag

SEQ ID NO: 150 - DNA, 95 bp, Artificial sequence
  Single strand DNA oligonucleotide
cuggaggagggucuccuuugggcgucgaggccucugcccaggacaccuucaugaccaguaggucuuggcgcucaacaggcggcugcuuacucucc

SEQ ID NO: 151 - DNA, 125 bp, Artificial sequence
  Single strand DNA oligonucleotide
cugacaccugggucagcaccacccagcgggaccugcagggaagaauccaugaugguccgaaucagggugagcauaaaauucgguaaauuguggcuucuacccucagaagccaauccggccgcgga

SEQ ID NO: 152 - DNA, 125 bp, Artificial sequence
  Single strand DNA oligonucleotide
ccccccccuccugccucgauucuuuaugcuggacgacuuggaaaguugaugggcuaguuggguagaccgagaggaggcuauucgugggguucacuaauccgccauuguucaaaacggucuucgag

SEQ ID NO: 153 - DNA, 82 bp, Artificial sequence
  Single strand DNA oligonucleotide
agcaaagagcagauuuuuuuagaucaagugauggggccaugacaaaaccccgucccacauuuuaucuaaaaaaaucugcuag

SEQ ID NO: 154 - DNA, 85 bp, Artificial sequence
  Single strand DNA oligonucleotide
cccuagcagagcuuuuguuugggacauugugguugguguccaaacuauccaaccacaauauuucaaaauaaagcuacuguuaggc

SEQ ID NO: 155 - DNA, 109 bp, Artificial sequence
  Single strand DNA oligonucleotide
ggccacagagcggcgcacgugacccgugugccccacauuuuaucuaaaaaaacgagaagagaugaaggggugucugguuggaauguggcgcgcaacagucaccucuggc

SEQ ID NO: 156 - DNA, 98 bp, Artificial sequence
  Single strand DNA oligonucleotide
guggggcccaggaggaccuauuggagggguaaugccauugcggugucaacgucaugucguggcaucacauuuccaacgggaaauccugggaaacccag

SEQ ID NO: 157 - DNA, 109 bp, Artificial sequence
  Single strand DNA oligonucleotide
uaccaacgcccaucgaacucgucacgagcgccagaaaugguaaagaaaauaaugagaacaaacgaaggauuguuucuaugcuauuucucgcgcuaaagacgacgcgugc

SEQ ID NO: 158 - DNA, 85 bp, Artificial sequence
  Single strand DNA oligonucleotide
ccuuagcagagcuguacaguggaaaauggagauuuauguccaaacuauuaaaucuccauauuccacuaaauagcuacuguuaggc

SEQ ID NO: 159 - DNA, 109 bp, Artificial sequence
  Single strand DNA oligonucleotide
gguaacugcccagcacaaggggcgcgugggcuugaaucauuuugaacuuuuaggagaacaaacgaaggcugcgguuuugggugauuuacgcccaaaagccuucgcgguc

SEQ ID NO: 160 - DNA, 82 bp, Artificial sequence
  Single strand DNA oligonucleotide
cgccgaggucaccggauuggugaagcccacagggucaaaggacagaagaagugauucgugggguucacuaauccgguggcgg

SEQ ID NO: 161 - DNA, 94 bp, Artificial sequence
  Single strand DNA oligonucleotide
cgcgccaggcacccagggcugaauucgaaguucaagcggaccguuaagggcuucggauuuggcacugccugccuggcgcgggacgaggcaacgg

SEQ ID NO: 162 - DNA, 85 bp, Artificial sequence
  Single strand DNA oligonucleotide
ccuuagcagagcugucguguuugacauuguguuguguguccaaacuaucacaacacaauuuugaacaacauagcuacuguuaggc

SEQ ID NO: 163 - DNA, 109 bp, Artificial sequence
  Single strand DNA oligonucleotide
ggagacuggcccacacaugugacuugugggcccgcuuccuauuucaaaaaaaugagaccacacgaacgagaguuugauguaggaggugcgcucaagugucaccccaggc

SEQ ID NO: 164 - DNA, 92 bp, Artificial sequence
  Single strand DNA oligonucleotide
agcaccgcaguggcugggauuagugaugccccucggauaccccaccagcggggggauucgugggguucacuaaucccagccaagccgguguc

SEQ ID NO: 165 - DNA, 23 bp, Artificial sequence
  Single strand DNA oligonucleotide
agtagctacatctggctactggg

SEQ ID NO: 166 - DNA, 23 bp, Artificial sequence
  Single strand DNA oligonucleotide
atttagaggtttatcgggagggg

SEQ ID NO: 167 - DNA, 23 bp, Artificial sequence
  Single strand DNA oligonucleotide
agtagctacatctggctactggg

SEQ ID NO: 168 - DNA, 23 bp, Artificial sequence
  Single strand DNA oligonucleotide
agagccctctgagcttcagcagg

SEQ ID NO: 169 - DNA, 23 bp, Artificial sequence
  Single strand DNA oligonucleotide
agtagctacatctggctactggg

SEQ ID NO: 170 - DNA, 23 bp, Artificial sequence
  Single strand DNA oligonucleotide
atttagaggtttatcgggagggg

SEQ ID NO: 171 - DNA, 23 bp, Artificial sequence
  Single strand DNA oligonucleotide
agtagctacatctggctactggg

SEQ ID NO: 172 - DNA, 23 bp, Artificial sequence
  Single strand DNA oligonucleotide
caacagctacattgtctgctggg

SEQ ID NO: 173 - DNA, 23 bp, Artificial sequence
  Single strand DNA oligonucleotide
agtagctacatctggctactggg

SEQ ID NO: 174 - DNA, 23 bp, Artificial sequence
  Single strand DNA oligonucleotide
cacgttgtctgtcaattcatagg

SEQ ID NO: 175 - DNA, 23 bp, Artificial sequence
  Single strand DNA oligonucleotide
agtagctacatctggctactggg

SEQ ID NO: 176 - DNA, 23 bp, Artificial sequence
  Single strand DNA oligonucleotide
aacccgtagatccgatcttgtgg

SEQ ID NO: 177 - DNA, 23 bp, Artificial sequence
  Single strand DNA oligonucleotide
agtagctacatctggctactggg

SEQ ID NO: 178 - DNA, 23 bp, Artificial sequence
  Single strand DNA oligonucleotide
atttagaggtttatcgggagggg

SEQ ID NO: 179 - DNA, 23 bp, Artificial sequence
  Single strand DNA oligonucleotide
aaagggtctcagggacgcagagg

SEQ ID NO: 180 - DNA, 23 bp, Artificial sequence
  Single strand DNA oligonucleotide
agagccctctgagcttcagcagg

SEQ ID NO: 181 - DNA, 23 bp, Artificial sequence
  Single strand DNA oligonucleotide
agtagctacatctggctactggg

SEQ ID NO: 182 - DNA, 23 bp, Artificial sequence
  Single strand DNA oligonucleotide
tgacagcgcattattactcacgg

SEQ ID NO: 183 - DNA, 23 bp, Artificial sequence
  Single strand DNA oligonucleotide
aaagggtctcagggacgcagagg

SEQ ID NO: 184 - DNA, 23 bp, Artificial sequence
  Single strand DNA oligonucleotide
cctctccagttccaagttacagg

SEQ ID NO: 185 - DNA, 23 bp, Artificial sequence
  Single strand DNA oligonucleotide
agtagctacatctggctactggg

SEQ ID NO: 186 - DNA, 23 bp, Artificial sequence
  Single strand DNA oligonucleotide
ggacccagttcaagtaattcagg

SEQ ID NO: 187 - DNA, 23 bp, Artificial sequence
  Single strand DNA oligonucleotide
aaagggtctcagggacgcagagg

SEQ ID NO: 188 - DNA, 23 bp, Artificial sequence
  Single strand DNA oligonucleotide
agagccctctgagcttcagcagg

SEQ ID NO: 189 - DNA, 23 bp, Artificial sequence
  Single strand DNA oligonucleotide
aagttgtattgttgtggggtagg

SEQ ID NO: 190 - DNA, 23 bp, Artificial sequence
  Single strand DNA oligonucleotide
cgactgtaaacatcctcgactgg

SEQ ID NO: 191 - DNA, 23 bp, Artificial sequence
  Single strand DNA oligonucleotide
aacccgtagatccgaacttgtgg

SEQ ID NO: 192 - DNA, 23 bp, Artificial sequence
  Single strand DNA oligonucleotide
attctgctcatgccagggtgagg

SEQ ID NO: 193 - DNA, 23 bp, Artificial sequence
  Single strand DNA oligonucleotide
aagttgtattgttgtggggtagg

SEQ ID NO: 194 - DNA, 23 bp, Artificial sequence
  Single strand DNA oligonucleotide
caactgtgtttcagctcagtagg

SEQ ID NO: 195 - DNA, 23 bp, Artificial sequence
  Single strand DNA oligonucleotide
aacccgtagatccgaacttgtgg

SEQ ID NO: 196 - DNA, 23 bp, Artificial sequence
  Single strand DNA oligonucleotide
attctgctcatgccagggtgagg

SEQ ID NO: 197 - DNA, 23 bp, Artificial sequence
  Single strand DNA oligonucleotide
aagttgtattgttgtggggtagg

SEQ ID NO: 198 - DNA, 23 bp, Artificial sequence
  Single strand DNA oligonucleotide
ccaagtaatggagaacaggctgg

SEQ ID NO: 199 - DNA, 23 bp, Artificial sequence
  Single strand DNA oligonucleotide
aacccgtagatccgaacttgtgg

SEQ ID NO: 200 - DNA, 23 bp, Artificial sequence
  Single strand DNA oligonucleotide
acagcagaatatcacacagctgg

SEQ ID NO: 201 - DNA, 23 bp, Artificial sequence
  Single strand DNA oligonucleotide
attctgctcatgccagggtgagg

SEQ ID NO: 202 - DNA, 23 bp, Artificial sequence
  Single strand DNA oligonucleotide
cactaaagtgcttatagtgcagg

SEQ ID NO: 203 - DNA, 23 bp, Artificial sequence
  Single strand DNA oligonucleotide
ggttgaggtagtaggttgtatgg

SEQ ID NO: 204 - DNA, 23 bp, Artificial sequence
  Single strand DNA oligonucleotide
aagttgtattgttgtggggtagg

SEQ ID NO: 205 - DNA, 23 bp, Artificial sequence
  Single strand DNA oligonucleotide
aagttgtattgttgtggggtagg

SEQ ID NO: 206 - DNA, 23 bp, Artificial sequence
  Single strand DNA oligonucleotide
cactaaagtgcttatagtgcagg

SEQ ID NO: 207 - DNA, 23 bp, Artificial sequence
  Single strand DNA oligonucleotide
aacccgtagatccgaacttgtgg

SEQ ID NO: 208 - DNA, 23 bp, Artificial sequence
  Single strand DNA oligonucleotide
aagttgtattgttgtggggtagg

SEQ ID NO: 209 - DNA, 23 bp, Artificial sequence
  Single strand DNA oligonucleotide
cggatccgtctgagcttggctgg

SEQ ID NO: 210 - DNA, 23 bp, Artificial sequence
  Single strand DNA oligonucleotide
acatcacaagttagggtctcagg

SEQ ID NO: 211 - DNA, 23 bp, Artificial sequence
  Single strand DNA oligonucleotide
cagataaagcgtacgctatacgg

SEQ ID NO: 212 - DNA, 23 bp, Artificial sequence
  Single strand DNA oligonucleotide
acttgaagagaagttgttcgtgg

SEQ ID NO: 213 - DNA, 23 bp, Artificial sequence
  Single strand DNA oligonucleotide
acttgaagagaagttgttcgtgg

SEQ ID NO: 214 - DNA, 23 bp, Artificial sequence
  Single strand DNA oligonucleotide
aagcactccgttcaccctctggg

SEQ ID NO: 215 - DNA, 23 bp, Artificial sequence
  Single strand DNA oligonucleotide
aacttctccttaagcaccacagg

SEQ ID NO: 216 - DNA, 23 bp, Artificial sequence
  Single strand DNA oligonucleotide
acttgaagagaagttgttcgtgg

SEQ ID NO: 217 - DNA, 23 bp, Artificial sequence
  Single strand DNA oligonucleotide
acttgaagagaagttgttcgtgg

SEQ ID NO: 218 - DNA, 23 bp, Artificial sequence
  Single strand DNA oligonucleotide
aagcactccgttcaccctctggg

SEQ ID NO: 219 - DNA, 23 bp, Artificial sequence
  Single strand DNA oligonucleotide
cagataaagcgtacgctatacgg

SEQ ID NO: 220 - DNA, 23 bp, Artificial sequence
  Single strand DNA oligonucleotide
acttgaagagaagttgttcgtgg

SEQ ID NO: 221 - DNA, 23 bp, Artificial sequence
  Single strand DNA oligonucleotide
acttgaagagaagttgttcgtgg

SEQ ID NO: 222 - DNA, 23 bp, Artificial sequence
  Single strand DNA oligonucleotide
agagtaagcagccgcctgtgagg

SEQ ID NO: 223 - DNA, 23 bp, Artificial sequence
  Single strand DNA oligonucleotide
ccatagtctaccatctctgcagg

SEQ ID NO: 224 - DNA, 23 bp, Artificial sequence
  Single strand DNA oligonucleotide
cagataaagcgtacgctatacgg

SEQ ID NO: 225 - DNA, 23 bp, Artificial sequence
  Single strand DNA oligonucleotide
agcccaaaaggagaattctttgg

SEQ ID NO: 226 - DNA, 23 bp, Artificial sequence
  Single strand DNA oligonucleotide
agagctgtggagtgtgacaatgg

SEQ ID NO: 227 - DNA, 23 bp, Artificial sequence
  Single strand DNA oligonucleotide
agcctccttcatatattctcagg

SEQ ID NO: 228 - DNA, 23 bp, Artificial sequence
  Single strand DNA oligonucleotide
agtactgtagcagcacatcatgg

SEQ ID NO: 229 - DNA, 23 bp, Artificial sequence
  Single strand DNA oligonucleotide
agcctccttcatatattctcagg

SEQ ID NO: 230 - DNA, 23 bp, Artificial sequence
  Single strand DNA oligonucleotide
agagctgtggagtgtgacaatgg

SEQ ID NO: 231 - DNA, 23 bp, Artificial sequence
  Single strand DNA oligonucleotide
agcctccttcatatattctcagg

SEQ ID NO: 232 - DNA, 23 bp, Artificial sequence
  Single strand DNA oligonucleotide
agcccaaaaggagaattctttgg

SEQ ID NO: 233 - DNA, 23 bp, Artificial sequence
  Single strand DNA oligonucleotide
acacttactgaacacctactagg

SEQ ID NO: 234 - DNA, 23 bp, Artificial sequence
  Single strand oligonucleotide
agagctgtggagtgtgacaatgg

SEQ ID NO: 235 - DNA, 23 bp, Artificial sequence
  Single strand DNA oligonucleotide
agcctccttcatatattctcagg

SEQ ID NO: 236 - DNA, 23 bp, Artificial sequence
  Single strand DNA oligonucleotide
ctcatggcaacagcagtcgatgg

SEQ ID NO: 237 - DNA, 22 bp, Artificial sequence
  Single strand DNA oligonucleotide
gttgagagtgttggagaaggag

SEQ ID NO: 238 - DNA, 21 bp, Artificial sequence
  Single strand DNA oligonucleotide
ctcggtgttgatcctgagaag

SEQ ID NO: 239 - DNA, 20 bp, Artificial sequence
  Single strand DNA oligonucleotide
gtactgctggtcctttgcag

SEQ ID NO: 240 - DNA, 20 bp, Artificial sequence
  Single strand DNA oligonucleotide
aggagcactacggaaggatg

SEQ ID NO: 241 - DNA, 19 bp, Artificial sequence
  Single strand DNA oligonucleotide
acaccctgggaattggttt

SEQ ID NO: 242 - DNA, 20 bp, Artificial sequence
  Single strand DNA oligonucleotide
gtatgcgccaataagaccac

SEQ ID NO: 243 - DNA, 999 bp, Artificial sequence
  Single strand  oligonucleotide
atgttttatgactaataaacttgaaaatgactaaacaataatcattaatcttgtcgagtatctgacaatgtggaggacagaagaaagggattgctgcctggtcaatgtgaggctttcctctaaaggtagtaccaagtcagactgccttcctgataacaggtctcattttgtaggacccttattgtgtgttttttggggaagcctactgtaaaagccaacattttaatgggattgtatcttatatttctttaaggagttttttttttttttttaaggcttacatgtgtccaatttggaacttctggctattggctcctcccctcccccccttgggtataagctgtaattgatgtttgtgacagaatttagagctttggctttttcctttttgtctaattatttatttcaaatttagcaggaataaagtgaacctcaccttgggactgaagctgtgaccagtcagaataatgcagttgtactccagcttgtgccagtgatgtgcgcatctacacaacttagggtacaggaagcattatgctgacagctgcctcggtgggagccacagtgggcgctgcctcgggcggcactggctgcgtccagtcgtcggtcagtcggtcgcggggagggcctgctggtgctgcgtgctttttgttctaaggtgcatctagtgcagatagtgaagtagactagcatctactgccctaagtgctccttctggcataagaagttatgtcctcatccaatccaagtcaagcaagcatgtaggggtctctccatagttgtgtttgcagccctctgttagttttgcatagttgcactacaagaagaatgtagttgtgcaaatctatgcaaaactgatggtggcctgctatttacttcaagtgttgtttttttttaaactaattttgtatttttattgtgtcgatgtagagcctgcgtggtgtgtgtgatgtgacagcttctgtagcactaaagtgcttatagtgcaggtagtgtgta

SEQ ID NO: 244 - DNA, 999 bp, Artificial sequence
  Single strand  oligonucleotide
atgttttatgactaataaacttgaaaatgactaaacaataatcattaatcttgtcgagtatctgacaatgtggaggacagaagaaagggattgctgcctggtcaatgtgaggctttcctctaaaggtagtaccaagtcagactgccttcctgataacaggtctcattttgtaggacccttattgtgtgttttttggggaagcctactgtaaaagccaacattttaatgggattgtatcttatatttctttaaggagttttttttttttttttaaggcttacatgtgtccaatttggaacttctggctattggctcctcccctcccccccttgggtataagctgtaattgatgtttgtgacagaatttagagctttggctttttcctttttgtctaattatttatttcaaatttagcaggaataaagtgaacctcaccttgggactgaagctgtgaccagtcagaataatgccttcatcatcgctaatcacgacgagatgtgtgcatcgagtgatgggaggtgatgactagcattatgctgacagctgcctcggtgggagccacagtgggcgctgcctcgggcggcactggctgcgtccagtcgtcggtcagtcggtcgcggggagggcctgctggtgctgcgtgctttttgttctaaggtgcatctagtgcagatagtgaagtagactagcatctactgccctaagtgctccttctggcataagaagttatgtcctcatccaatccaagtcaagcaagcatgtaggggtctctccatagttgtgtttgcagccctctgttagttttgcatagttgcactacaagaagaatgtagttgtgcaaatctatgcaaaactgatggtggcctgctatttacttcaagtgttgtttttttttaaactaattttgtatttttattgtgtcgatgtagagcctgcgtggtgtgtgtgatgtgacagcttctgtagcactaaagtgcttatagtgcaggtagtgtgta

SEQ ID NO: 245 - DNA, 999 bp, Artificial sequence
  Single strand  oligonucleotide
atgttttatgactaataaacttgaaaatgactaaacaataatcattaatcttgtcgagtatctgacaatgtggaggacagaagaaagggattgctgcctggtcaatgtgaggctttcctctaaaggtagtaccaagtcagactgccttcctgataacaggtctcattttgtaggacccttattgtgtgttttttggggaagcctactgtaaaagccaacattttaatgggattgtatcttatatttctttaaggagttttttttttttttttaaggcttacatgtgtccaatttggaacttctggctattggctcctcccctcccccccttgggtataagctgtaattgatgtttgtgacagaatttagagctttggctttttcctttttgtctaattatttatttcaaatttagcaggaataaagtgaacctcaccttgggactgaagctgtgaccagtcagaataatgtcataatccagcaggtcagcaaagtgatgtgcgcatctagctgagttactggattggtagcattatgctgacagctgcctcggtgggagccacagtgggcgctgcctcgggcggcactggctgcgtccagtcgtcggtcagtcggtcgcggggagggcctgctggtgctgcgtgctttttgttctaaggtgcatctagtgcagatagtgaagtagactagcatctactgccctaagtgctccttctggcataagaagttatgtcctcatccaatccaagtcaagcaagcatgtaggggtctctccatagttgtgtttgcagccctctgttagttttgcatagttgcactacaagaagaatgtagttgtgcaaatctatgcaaaactgatggtggcctgctatttacttcaagtgttgtttttttttaaactaattttgtatttttattgtgtcgatgtagagcctgcgtggtgtgtgtgatgtgacagcttctgtagcactaaagtgcttatagtgcaggtagtgtgta

SEQ ID NO: 246 - DNA, 999 bp, Artificial sequence
  Single strand  oligonucleotide
atgttttatgactaataaacttgaaaatgactaaacaataatcattaatcttgtcgagtatctgacaatgtggaggacagaagaaagggattgctgcctggtcaatgtgaggctttcctctaaaggtagtaccaagtcagactgccttcctgataacaggtctcattttgtaggacccttattgtgtgttttttggggaagcctactgtaaaagccaacattttaatgggattgtatcttatatttctttaaggagttttttttttttttttaaggcttacatgtgtccaatttggaacttctggctattggctcctcccctcccccccttgggtataagctgtaattgatgtttgtgacagaatttagagctttggctttttcctttttgtctaattatttatttcaaatttagcaggaataaagtgaacctcaccttgggactgaagctgtgaccagtcagaataatgtcagtacgtgcacataacagactgtgatgtgcgcatctactgttttgagcgcgtgcgtagcattatgctgacagctgcctcggtgggagccacagtgggcgctgcctcgggcggcactggctgcgtccagtcgtcggtcagtcggtcgcggggagggcctgctggtgctgcgtgctttttgttctaaggtgcatctagtgcagatagtgaagtagactagcatctactgccctaagtgctccttctggcataagaagttatgtcctcatccaatccaagtcaagcaagcatgtaggggtctctccatagttgtgtttgcagccctctgttagttttgcatagttgcactacaagaagaatgtagttgtgcaaatctatgcaaaactgatggtggcctgctatttacttcaagtgttgtttttttttaaactaattttgtatttttattgtgtcgatgtagagcctgcgtggtgtgtgtgatgtgacagcttctgtagcactaaagtgcttatagtgcaggtagtgtgta

SEQ ID NO: 247 - DNA, 999 bp, Artificial sequence
  Single strand  oligonucleotide
atgttttatgactaataaacttgaaaatgactaaacaataatcattaatcttgtcgagtatctgacaatgtggaggacagaagaaagggattgctgcctggtcaatgtgaggctttcctctaaaggtagtaccaagtcagactgccttcctgataacaggtctcattttgtaggacccttattgtgtgttttttggggaagcctactgtaaaagccaacattttaatgggattgtatcttatatttctttaaggagttttttttttttttttaaggcttacatgtgtccaatttggaacttctggctattggctcctcccctcccccccttgggtataagctgtaattgatgtttgtgacagaatttagagctttggctttttcctttttgtctaattatttatttcaaatttagcaggaataaagtgaacctcaccttgggactgaagctgtgaccagtcagaataatgtcagaaggttcccactggagtctgtgatgagtgcatcttctccaatgagggcctttgtagcattatgctgacagctgcctcggtgggagccacagtgggcgctgcctcgggcggcactggctgcgtccagtcgtcggtcagtcggtcgcggggagggcctgctggtgctgcgtgctttttgttctaaggtgcatctagtgcagatagtgaagtagactagcatctactgccctaagtgctccttctggcataagaagttatgtcctcatccaatccaagtcaagcaagcatgtaggggtctctccatagttgtgtttgcagccctctgttagttttgcatagttgcactacaagaagaatgtagttgtgcaaatctatgcaaaactgatggtggcctgctatttacttcaagtgttgtttttttttaaactaattttgtatttttattgtgtcgatgtagagcctgcgtggtgtgtgtgatgtgacagcttctgtagcactaaagtgcttatagtgcaggtagtgtgta

SEQ ID NO: 248 - DNA, 999 bp, Artificial sequence
  Single strand  oligonucleotide
tccgctcccacacaactagagaatactcaggttctgatcggaccagaggtatcacttcagaatagagttttatttattcatttatttgttcgtttgtttgtttgtttatttttaaagtaacaattaaacccgtaacgaaaagttcataccggtccattgtttaacaatcagggacatttctttttttgtatttggttggtcggttggttttcggttgtttcctgttggaaaggttttaggtactccgttccactgaaaagtaaaaataaataaatcaatactgacactgctgatggggttaaaggatccttcaaaagagaactatgacgacgacaacatggttccatattcccgaggttcagagtgttctgtattcctggtgttaaaaactgacgtttggtactacgacccattacaaacttacttttgtaaactatacctaccagtctactttggtaccaaattgtcagtaagccacgcgcctcctgtagccatgagcttcaacggtccacatgaagcagcacgacttaccgacacggtggtacgcggtacgctacagtgctggtgctgtcccccgttcgtgctccacgagtccgtcccaaatttcgtttcgtttgtagagaccaaacgaccccgaattacggttaagatcctttctttcgacaaataaaaggatggtccgttttgttttacttgtcccagttagtttttttacagtttccttttagtgtaactttattttgtgtctttagtgtgtctcttcattcgaaggtggacaatttcggcctcatgtttgtcgattttgattgtacttgtactttttcggaaagaacgatcacaggagactaaatatgtagtggtgaagaacaaaaatatttcattctctaagggtgagatccacttggagtatcagggcgaatctagaggtcgggttgttgttttgtggaacctccatcgagagagtccgttatgtattttcggacccgttaagaccaaatt

SEQ ID NO: 249 - DNA, 999 bp, Artificial sequence
  Single strand  oligonucleotide
tccgctcccacacaactagagaatactcaggttctgatcggaccagaggtatcacttcagaatagagttttatttattcatttatttgttcgtttgtttgtttgtttatttttaaagtaacaattaaacccgtaacgaaaagttcataccggtccattgtttaacaatcagggacatttctttttttgtatttggttggtcggttggttttcggttgtttcctgttggaaaggttttaggtactccgttccactgaaaagtaaaaataaataaatcaatactgacactgctgatggggttaaaggatccttcaaaagagaactatgacgacgacaacatggttccatattcccgaggttcagagtgttctgtattcctggtgttaaaaactgacgtttggtactacgacccattacaaacttacttttgtaaactatacctaccagtctactttgataccatactgtcagactcaccatcctaactacgacccatgagattcaacagtgtcgtgattagcgatgatgaatccgacacggtggtacacggtacgctacagtgctggtgctgtcccccgttcgtgctccacgagtccgtcccaaatttcgtttcgtttgtagagaccaaacgaccccgaattacggttaagatcctttctttcgacaaataaaaggatggtccgttttgttttacttgtcccagttagtttttttacagtttccttttagtgtaactttattttgtgtctttagtgtgtctcttcattcgaaggtggacaatttcggcctcatgtttgtcgattttgattgtacttgtactttttcggaaagaacgatcacaggagactaaatatgtagtggtgaagaacaaaaatatttcattctctaagggtgagatccacttggagtatcagggcgaatctagaggtcgggttgttgttttgtggaacctccatcgagagagtccgttatgtattttcggacccgttaagaccaaatt

SEQ ID NO: 250 - DNA, 999 bp, Artificial sequence
  Single strand  oligonucleotide
tccgctcccacacaactagagaatactcaggttctgatcggaccagaggtatcacttcagaatagagttttatttattcatttatttgttcgtttgtttgtttgtttatttttaaagtaacaattaaacccgtaacgaaaagttcataccggtccattgtttaacaatcagggacatttctttttttgtatttggttggtcggttggttttcggttgtttcctgttggaaaggttttaggtactccgttccactgaaaagtaaaaataaataaatcaatactgacactgctgatggggttaaaggatccttcaaaagagaactatgacgacgacaacatggttccatattcccgaggttcagagtgttctgtattcctggtgttaaaaactgacgtttggtactacgacccattacaaacttacttttgtaaactatacctaccagtctactttgttaccataatgtcggacaacccaacgaccaccaaagccatgagattcaacagtctttgctgacctgctggattatccgacaaggtggtaagcggtacgctacagtgctggtgctgtcccccgttcgtgctccacgagtccgtcccaaatttcgtttcgtttgtagagaccaaacgaccccgaattacggttaagatcctttctttcgacaaataaaaggatggtccgttttgttttacttgtcccagttagtttttttacagtttccttttagtgtaactttattttgtgtctttagtgtgtctcttcattcgaaggtggacaatttcggcctcatgtttgtcgattttgattgtacttgtactttttcggaaagaacgatcacaggagactaaatatgtagtggtgaagaacaaaaatatttcattctctaagggtgagatccacttggagtatcagggcgaatctagaggtcgggttgttgttttgtggaacctccatcgagagagtccgttatgtattttcggacccgttaagaccaaatt

SEQ ID NO: 251 - DNA, 999 bp, Artificial sequence
  Single strand  oligonucleotide
tccgctcccacacaactagagaatactcaggttctgatcggaccagaggtatcacttcagaatagagttttatttattcatttatttgttcgtttgtttgtttgtttatttttaaagtaacaattaaacccgtaacgaaaagttcataccggtccattgtttaacaatcagggacatttctttttttgtatttggttggtcggttggttttcggttgtttcctgttggaaaggttttaggtactccgttccactgaaaagtaaaaataaataaatcaatactgacactgctgatggggttaaaggatccttcaaaagagaactatgacgacgacaacatggttccatattcccgaggttcagagtgttctgtattcctggtgttaaaaactgacgtttggtactacgacccattacaaacttacttttgtaaactatacctaccagtctactttgttaccaaactgtcagacagcacgtaacataccagaccaatgagattcaactgagtctgttatgtgcacgtactatccgacacggtggtaaacggtacgctacagtgctggtgctgtcccccgttcgtgctccacgagtccgtcccaaatttcgtttcgtttgtagagaccaaacgaccccgaattacggttaagatcctttctttcgacaaataaaaggatggtccgttttgttttacttgtcccagttagtttttttacagtttccttttagtgtaactttattttgtgtctttagtgtgtctcttcattcgaaggtggacaatttcggcctcatgtttgtcgattttgattgtacttgtactttttcggaaagaacgatcacaggagactaaatatgtagtggtgaagaacaaaaatatttcattctctaagggtgagatccacttggagtatcagggcgaatctagaggtcgggttgttgttttgtggaacctccatcgagagagtccgttatgtattttcggacccgttaagaccaaatt

SEQ ID NO: 252 - DNA, 999 bp, Artificial sequence
  Single strand  oligonucleotide
tccgctcccacacaactagagaatactcaggttctgatcggaccagaggtatcacttcagaatagagttttatttattcatttatttgttcgtttgtttgtttgtttatttttaaagtaacaattaaacccgtaacgaaaagttcataccggtccattgtttaacaatcagggacatttctttttttgtatttggttggtcggttggttttcggttgtttcctgttggaaaggttttaggtactccgttccactgaaaagtaaaaataaataaatcaatactgacactgctgatggggttaaaggatccttcaaaagagaactatgacgacgacaacatggttccatattcccgaggttcagagtgttctgtattcctggtgttaaaaactgacgtttggtactacgacccattacaaacttacttttgtaaactatacctaccagtctactttgataccaaaatgtcagatagaaggttccacttgagtcccgtgagattcaacggagactccagtgggaaccttctatccgacaaggtggtacacggtacgctacagtgctggtgctgtcccccgttcgtgctccacgagtccgtcccaaatttcgtttcgtttgtagagaccaaacgaccccgaattacggttaagatcctttctttcgacaaataaaaggatggtccgttttgttttacttgtcccagttagtttttttacagtttccttttagtgtaactttattttgtgtctttagtgtgtctcttcattcgaaggtggacaatttcggcctcatgtttgtcgattttgattgtacttgtactttttcggaaagaacgatcacaggagactaaatatgtagtggtgaagaacaaaaatatttcattctctaagggtgagatccacttggagtatcagggcgaatctagaggtcgggttgttgttttgtggaacctccatcgagagagtccgttatgtattttcggacccgttaagaccaaatt

SEQ ID NO: 253 - DNA, 80 bp, Artificial sequence
  Single strand  oligonucleotide
cctgttgccacaaacccgtagatccgaacttgtggtattagtccgcacaagcttgtatctataggtatgtgtctgttagg

SEQ ID NO: 254 - DNA, 80 bp, Artificial sequence
  Single strand  oligonucleotide
cctgttgccaatattgcgcagatcggtacttgtggtagaagtccgcacaagcatttgtttgtacaagattgtaggttagg

SEQ ID NO: 255 - DNA, 80 bp, Artificial sequence
  Single strand  oligonucleotide
cgttaagaccggtttgctgagtcacgcaagtcttgcagcagtccgaagattcgtggggttcactaatccggaagagggcg

SEQ ID NO: 256 - DNA, 80 bp, Artificial sequence; identical to SEQ ID NO: 253
  Single strand  oligonucleotide

SEQ ID NO: 257 - DNA, 80 bp, Artificial sequence
  Single strand  oligonucleotide
cctgtagccacaaattcgtggggttcactaatccgtacgagtccgggattgctggctcctataggtatgtgcccgttagg

SEQ ID NO: 258 - DNA, 80 bp, Artificial sequence
  Single strand  oligonucleotide
ccggattctacaatggattcgccattttatttttgaattaggcaaacaggtcgtgctgggagaacgatgtaccagcccgg

SEQ ID NO: 259 - DNA, 94 bp, Artificial sequence
  Single strand  oligonucleotide
aacaacagaccacaccatggttccactggggcttgaacccaggaccttctgcgtgtaaagcagatgtgataaccactacactatggaaccacag

SEQ ID NO: 260 - DNA, 94 bp, Artificial sequence
  Single strand  oligonucleotide
aacaacagaccacaccatggtttcactggggcttgaacccaggaccatctgcgagtaaagcagaattcacggagaagacaggatgaaaccacag

SEQ ID NO: 261 - DNA, 94 bp, Artificial sequence
  Single strand  oligonucleotide
aacaacagactatccagggggttgggagagggcagtactctcgatcgtcagcgtgtaaagctgagtacaattcgtggggttcactaatcccgag

SEQ ID NO: 262 - DNA, 94 bp, Artificial sequence; identical to SEQ ID NO: 259
  Single strand  oligonucleotide

SEQ ID NO: 263 - DNA, 94 bp, Artificial sequence
  Single strand  oligonucleotide
aacaacagacaacaccatgggtcttctggggcctgaacccaggaccgtctgcgtgtaaagcagaattgttcatcaacgaggaagagattcacag

SEQ ID NO: 264 - DNA, 94 bp, Artificial sequence
  Single strand  oligonucleotide
aacaacagaccgcaccatggtttctgtggggccggaacccattattttcggcgagaaaagctgattggattcatcaacgaggaagagattacag

SEQ ID NO: 265 - DNA, 416 bp, Artificial sequence
  Single strand  oligonucleotide
aaaacgtaataagttctttttgtgtgtgtctgcaggcaatatcaaaaacataaccatcatgatgtatagagactgtcgggtccattgtgaggagacattcagtttctctttaaaactccttcattgaaatagtccggtgttatccctacctgagcttagttttttttttttaattttttttctgtcctattgaattattctattttcttgaccttgtaagacccatccctttcaaagtatctcaaccttctatcgttttaaagactctctcctatctctttttggtgttgagtatgtgtgtatctctactcctagttcatttgaatcagtttttctaccttgtctatccctcctgagctaatgtttgcatcttcttgttggtcattgatgtatggttgatataaattccaaataaa

SEQ ID NO: 266 - DNA, 416 bp, Artificial sequence
  Single strand  oligonucleotide
aaaacgtaataagttctttttgtgtgtgtctgcaggcaatatcaaaaacataaccatcatgatgtatagagactgtcgggtccattgtgaggagacattcagtttctctttaaaactccttcattgaaatagtccggtgttatccctacctgagcttagttttttttttttaattttttttctgtcctattgaattattctattctcttgtccatgttcgacccatccctttcaaagtatctcaaccttctatcgttttaaagactctctcctatctctttttggtgttgagtatgtgtgtatctctactcctagttcatttgaatcagtttttctaccttgtctatccctcctgagctaatgtttgcatcttcttgttggtcattgatgtatggttgatataaattccaaataaa

SEQ ID NO: 267 - DNA, 416 bp, Artificial sequence
  Single strand  oligonucleotide
aaaacgtaataagttctttttgtgtgtgtctgcaggcaatatcaaaaacataaccatcatgatgtatagagactgtcgggtccattgtgaggagacattcagtttctctttaaaactccttcattgaaatagtccggtgttatccctacctgagcttagttttttttttttaattttttttctgtcctattgaattattctattggtcacttgaccgccatgacatccctttcaaagtatctcaaccttctatcgttttaaagactctctcctatctctttttggtgttgagtatgtgtgtatctctactcctagttcatttgaatcagtttttctaccttgtctatccctcctgagctaatgtttgcatcttcttgttggtcattgatgtatggttgatataaattccaaataaa

(19) United States
(12) Patent Application Publication
(10) Pub. No.: US 2022/0186219 A1
(43) Pub. Date: Jun. 16, 2022

(54) MODIFYING THE SPECIFICITY OF NON-CODING RNA MOLECULES FOR SILENCING GENES IN EUKARYOTIC CELLS

(71) Applicant: Tropic Biosciences UK Limited, Colney, Norwich (GB)

(72) Inventors: Eyal MAORI, Cambridge (GB); Yaron GALANTY, Coton, Cambridge (GB); Cristina PIGNOCCHI, Hethersett, Norwich (GB); Angela CHAPARRO GARCIA, Norwich (GB); Ofir MEIR, Norwich, Norfolk (GB)

(73) Assignee: Tropic Biosciences UK Limited, Colney, Norwich (GB)

(21) Appl. No.: 17/438,920

(22) PCT Filed: Mar. 12, 2020
(86) PCT No.: PCT/IB2020/052241

§ 371 Date: Sep. 13, 2021

(30) Foreign Application Priority Data
Mar. 14, 2019 (GB) 1903520.3

(51) Int. Cl. C12N 15/113; C12N 15/11

(52) U.S. Cl. CPC C12N 15/113; C12N 15/111; C12N 2310/20; C12N 2310/141; C12N 2310/113

(57) ABSTRACT

A method of modifying a gene encoding or processed into a non-coding RNA molecule having no RNA silencing activity in a eukaryotic cell, wherein the gene encoding or processed into the non-coding RNA molecule is positioned in a coding gene, is disclosed. The method comprising introducing into the eukaryotic cell a DNA editing agent conferring a silencing specificity of the non-coding RNA molecule towards a target RNA of interest. Target RNA of interest include, for example, a transcript of a gene selected from the group consisting of a housekeeping gene, a dominant gene, a gene comprising a high copy number, and a gene associated with cell apoptosis. Methods comprising DNA or RNA editing agents which elicit base editing are also disclosed.

### RELATED APPLICATION/S

This application claims the benefit of priority of UK Patent Application No. 1903520.3 filed on 14 Mar. 2019, the contents of which are incorporated herein by reference in their entirety.

### SEQUENCE LISTING STATEMENT

The ASCII file, entitled 81323SequenceListing.docx, created on 11 Mar. 2020, comprising 86,492 bytes, submitted concurrently with the filing of this application is incorporated herein by reference.

### FIELD AND BACKGROUND OF THE INVENTION

The present invention, in some embodiments thereof, relates to modifying genes that encode or are processed into non-coding RNA molecules, including RNA silencing molecules and, more particularly, but not exclusively, to the use of same for silencing endogenous or exogenous target RNA of interest in eukaryotic cells.

Among the approximately 25,000 annotated genes in the human genome, mutations in over 3,000 have already been linked to disease phenotypes and more disease relevant genetic variations are being uncovered at a staggering pace. Emerging therapeutic strategies that can modify nucleic acids within disease-affected cells and tissues have potential for the treatment of monogenic, highly penetrant diseases, such as Severe Combined Immunodeficiency (SCID), hemophilia and certain enzyme deficiencies, owing to their well-defined genetics and often lack of safe, effective alternative treatments. Two of the most powerful genetic therapeutic technologies developed thus far are gene therapy, which enables restoration of missing gene function by viral transgene expression, and RNA interference (RNAi), which mediates repression of defective genes by knockdown of the target mRNA.

Gene therapy has been used to successfully treat monogenic recessive disorders affecting the hematopoietic system, such as SCID and Wiskott-Aldrich syndrome, by semi-randomly integrating functional genes into the genome of hematopoietic stem/progenitor cells [Gaspar et al., Sci. Transl. Med. (2011) 3: 97ra79; Howe et al., J. Clin. Invest. (2008) 118: 3143-3150]. RNAi has been used to repress the function of genes implicated in cancer, age-related macular degeneration and transthyretin (TTR)-related amyloidosis, among others in clinical trials. Despite promise and recent success, gene therapy and RNAi have limitations that preclude their utility for a large number of diseases. For example, viral gene therapy may cause mutagenesis at the integration site and result in dysregulated transgene expression [Howe et al. (2008), supra]. Meanwhile, the use of RNAi is limited to targets for which gene knockdown is beneficial. Also, RNAi often cannot fully repress gene expression due to the transient nature of the delivered siRNA and the lack of silencing amplification mechanisms, for example in plants or nematodes, and is therefore unlikely to provide a benefit for diseases in which complete repression of gene function is necessary for therapy. The current main obstacle of RNA-based therapeutics is efficient and effective RNA delivery into cells. Although some delivery agents can enhance therapeutic RNA endocytosis, only a very small fraction, less than 0.01%, escapes from the endosomes and is biologically active [Steven F Dowdy, Nature Biotechnol (2017) 35, 222-229].

Recent advances in genome editing techniques have made it possible to alter DNA sequences in living cells by editing only a few of the billions of nucleotides in the cells of human patients. In the past decade, the tools and expertise for using genome editing in human somatic cells and pluripotent cells have increased to such an extent that the approach is now being developed widely as a strategy to treat human disease. The fundamental process depends on creating a site-specific DNA double-strand break (DSB) in the genome and then allowing the cell's endogenous DSB repair machinery to fix the break (such as by non-homologous end-joining (NHEJ) or homologous recombination (HR)) in which the latter can allow precise nucleotide changes to be made to the DNA sequence [Porteus, Annu Rev Pharmacol Toxicol. (2016) 56:163-90].

Three primary approaches use mutagenic genome editing (NHEJ) of cells as potential therapeutics: (a) knocking out functional genetic elements by creating spatially precise insertions or deletions, (b) creating insertions or deletions that compensate for underlying frameshift mutations; hence reactivating partly- or non-functional genes, and (c) creating defined genetic deletions. Although several different therapeutic applications use editing by NHEJ, genome editing by homologous recombination (HR) will most likely offer the broadest application scope. This is because HR, although a rare event, is highly accurate as it relies on an exogenously provided template to copy a specific, predetermined sequence during the repair process.

Currently the four major types of therapeutic applications to HR-mediated genome editing are: (a) gene correction (i.e. correction of diseases that are caused by point mutations in single genes), (b) functional gene correction (i.e. correction of diseases that are caused by mutations scattered throughout the gene), (c) safe harbor gene addition (i.e. when precise regulation is not required or when non-physiological levels of a therapeutic transgene are desired), and (d) targeted transgene addition (i.e. when precise regulation is required) [Porteus (2016), supra].

Previous work on genome editing of RNA molecules in various eukaryotic organisms (e.g. murine, human, shrimp, plants), focused on knocking-out miRNA gene activity or changing their binding site in target RNAs, for example:

With regard to genome editing in human cells, Jiang et al. [Jiang et al., RNA Biology (2014) 11 (10): 1243-9] used CRISPR/Cas9 to delete human miR-93 from a cluster by targeting its 5′ region in HeLa cells. Various small indels were induced in the targeted region containing the Drosha processing site (i.e. the position at which Drosha, a double-stranded RNA-specific RNase III enzyme, binds, cleaves and thereby processes primary miRNAs (pri-miRNAs) into pre-miRNA in the nucleus of a host cell) and seed sequences (i.e. the conserved heptametrical sequences which are essential for the binding of the miRNA to mRNA, typically situated at positions 2-7 from the miRNA 5′-end). According to Jiang et al. even a single nucleotide deletion led to complete knockout of the target miRNA with high specificity.

With regard to genome editing in murine species, Zhao et al. [Zhao et al., Scientific Reports (2014) 4:3943] provided a miRNA inhibition strategy employing the CRISPR system in murine cells. Zhao used specifically designed sgRNAs to cut miRNA gene at a single site by Cas9, resulting in knockout of the miRNA in these cells.

With regard to plant genome editing, Bortesi and Fischer [Bortesi and Fischer, Biotechnology Advances (2015) 33: 41-52] discussed the use of CRISPR-Cas technology in plants as compared to ZFNs and TALENs, and Basak and Nithin [Basak and Nithin, Front Plant Sci. (2015) 6: 1001] teach that CRISPR-Cas technology has been applied for knockdown of protein-coding genes in model plants such as Arabidopsis and tobacco and crops like wheat, maize, and rice.

In addition to disruption of miRNA activity or target binding sites, gene silencing using artificial microRNAs (amiRNAs) mediated gene silencing of endogenous and exogenous target genes were used [Tiwari et al. Plant Mol Biol (2014) 86: 1]. Similar to microRNAs, amiRNAs are single-stranded, approximately 21 nucleotides (nt) long, and designed by replacing the mature miRNA sequences of duplex within pre-miRNAs [Tiwari et al. (2014) supra]. These amiRNAs are introduced as a transgene within an artificial expression cassette (including a promoter, terminator etc.) [Carbonell et al., Plant Physiology (2014) pp. 113.234989], are processed via small RNA biogenesis and silencing machinery and downregulate target expression. According to Schwab et al. [Schwab et al. The Plant Cell (2006) Vol. 18, 1121-1133], amiRNAs are active when expressed under tissue-specific or inducible promoters and can be used for specific gene silencing in plants, especially when several related, but not identical, target genes need to be downregulated.

Senis et al. [Senis et al., Nucleic Acids Research (2017) Vol. 45(1): e3] disclose engineering of a promoterless anti-viral RNAi hairpin into an endogenous miRNA locus. Specifically, Senis et al. insert an amiRNA precursor transgene (hairpin pri-amiRNA) adjacent to a naturally occurring miRNA gene (e.g. miR122) by homology-directed DNA recombination that is induced by sequence-specific nuclease such as Cas9 or TALEN. This approach uses promoter- and terminator-free amiRNAs by utilizing transcriptionally active DNA that expresses natural miRNA (miR122), that is, the endogenous promoter and terminator drove and regulated the transcription of the inserted amiRNA transgene.

Various DNA-free methods of introducing RNA and/or proteins into cells have been previously described. For example, RNA transfection using electroporation and lipofection has been described in U.S. Patent Application No. 20160289675. Direct delivery of Cas9/gRNA ribonucleoprotein (RNP) complexes to cells by microinjection of Cas9 protein and gRNA complexes was described by Cho [Cho et al., “Heritable gene knockout in Caenorhabditis elegans by direct injection of Cas9-sgRNA ribonucleoproteins,” Genetics (2013) 195:1177-1180]. Delivery of Cas9 protein/gRNA complexes via electroporation was described by Kim [Kim et al., “Highly efficient RNA-guided genome editing in human cells via delivery of purified Cas9 ribonucleoproteins” Genome Res. (2014) 24:1012-1019]. Delivery of Cas9 protein-associated sgRNA complexes via liposomes was reported by Zuris [Zuris et al., “Cationic lipid-mediated delivery of proteins enables efficient protein-based genome editing in vitro and in vivo” Nat Biotechnol. (2014) doi: 10.1038/nbt.3081].

### SUMMARY OF THE INVENTION

According to an aspect of some embodiments of the present invention there is provided a method of modifying a gene encoding or processed into a non-coding RNA molecule having no RNA silencing activity in a eukaryotic cell, wherein the gene encoding or processed into the non-coding RNA molecule is positioned in a coding gene, the method comprising introducing into the eukaryotic cell a DNA editing agent conferring a silencing specificity of the non-coding RNA molecule towards a target RNA of interest, thereby modifying the gene encoding or processed into the non-coding RNA molecule.

According to an aspect of some embodiments of the present invention there is provided a method of modifying a gene encoding or processed into a RNA silencing molecule to a target RNA in a eukaryotic cell, wherein the gene encoding or processed into the non-coding RNA molecule is positioned in a coding gene, the method comprising introducing into the eukaryotic cell a DNA editing agent which redirects a silencing specificity of the RNA silencing molecule towards a second target RNA, the target RNA and the second target RNA being distinct, thereby modifying the gene encoding or processed into the RNA silencing molecule.

According to an aspect of some embodiments of the present invention there is provided a method of modifying a gene encoding or processed into a non-coding RNA molecule having no RNA silencing activity in a eukaryotic cell, the method comprising introducing into the eukaryotic cell a DNA or RNA editing agent conferring a silencing specificity of the non-coding RNA molecule towards a target RNA of interest, wherein the DNA or RNA editing agent elicits base editing, thereby modifying the gene encoding or processed into the non-coding RNA molecule.

According to an aspect of some embodiments of the present invention there is provided a method of modifying a gene encoding or processed into a RNA silencing molecule to a target RNA in a eukaryotic cell, the method comprising introducing into the eukaryotic cell a DNA or RNA editing agent which redirects a silencing specificity of the RNA silencing molecule towards a second target RNA, the target RNA and the second target RNA being distinct, and wherein the DNA or RNA editing agent elicits base editing, thereby modifying the gene encoding or processed into the RNA silencing molecule.

According to an aspect of some embodiments of the present invention there is provided a method of modifying a gene encoding or processed into a non-coding RNA molecule having no RNA silencing activity in a eukaryotic cell, the method comprising introducing into the eukaryotic cell a DNA editing agent conferring a silencing specificity of the non-coding RNA molecule towards a target RNA of interest, wherein the target RNA of interest is a transcript of a gene selected from the group consisting of a housekeeping gene, a dominant gene, a gene comprising a high copy number, and a gene associated with cell apoptosis, thereby modifying the gene encoding or processed into the non-coding RNA molecule.

According to an aspect of some embodiments of the present invention there is provided a method of modifying a gene encoding or processed into a RNA silencing molecule to a target RNA in a eukaryotic cell, the method comprising introducing into the eukaryotic cell a DNA editing agent which redirects a silencing specificity of the RNA silencing molecule towards a second target RNA, wherein the second target RNA is a transcript of a gene selected from the group consisting of a housekeeping gene, a dominant gene, a gene comprising a high copy number, and a gene associated with cell apoptosis, the target RNA and the second target RNA being distinct, thereby modifying the gene encoding or processed into the RNA silencing molecule.

According to an aspect of some embodiments of the present invention there is provided a plant cell generated according to the method of some embodiments of the invention.

According to an aspect of some embodiments of the present invention there is provided a plant comprising the plant cell of some embodiments of the invention.

According to an aspect of some embodiments of the present invention there is provided a method of producing a plant comprising a reduced expression of a housekeeping gene, a dominant gene, a gene comprising a high copy number and/or a gene associated with cell apoptosis, the method comprising:

(a) breeding the plant of some embodiments of the invention; and

(b) selecting for progeny plants that have reduced expression of the housekeeping gene, the dominant gene, the gene comprising a high copy number, and/or gene associated with cell apoptosis, and which do not comprise the DNA editing agent, thereby producing the plant with reduced expression of the housekeeping gene, the dominant gene, the gene comprising a high copy number and/or gene associated with cell apoptosis.

According to an aspect of some embodiments of the present invention there is provided a method producing a plant or plant cell of some embodiments of the invention comprising growing the plant or plant cell under conditions which allow propagation.

According to an aspect of some embodiments of the present invention there is provided a seed of the plant of some embodiments of the invention.

According to an aspect of some embodiments of the present invention there is provided a method of treating a disease in a subject in need thereof, the method comprising modifying a gene encoding or processed into a non-coding RNA molecule or into an RNA silencing molecule according to the method of some embodiments of the invention, wherein the target RNA of interest or the second target RNA is a transcript of a housekeeping gene, a dominant gene, a gene comprising a high copy number, and/or a gene associated with cell apoptosis, associated with an onset or progression of the disease.

According to some embodiments of the invention, the gene encoding or processed into the non-coding RNA molecule or into the RNA silencing molecule is positioned in a non-coding gene.

According to some embodiments of the invention, the gene encoding or processed into the non-coding RNA molecule or into the RNA silencing molecule is positioned in a coding gene.

According to some embodiments of the invention, the gene encoding or processed into the non-coding RNA molecule or into the RNA silencing molecule is positioned within an exon of coding gene.

According to some embodiments of the invention, the gene encoding or processed into the non-coding RNA molecule or into the RNA silencing molecule is positioned within an exon encoding an untranslated region (UTR) of a coding gene.

According to some embodiments of the invention, the gene encoding or processed into the non-coding RNA molecule or into the RNA silencing molecule is positioned within an intron of coding gene.

According to some embodiments of the invention, the gene encoding or processed into the non-coding RNA molecule or into the RNA silencing molecule is endogenous to the eukaryotic cell.

According to some embodiments of the invention, the modifying the gene encoding or processed into the non-coding RNA molecule comprises imparting the non-coding RNA molecule with at least 45% complementarity towards the target RNA of interest.

According to some embodiments of the invention, the modifying the gene encoding or processed into the RNA silencing molecule comprises imparting the RNA silencing molecule with at least 45% complementarity towards the second target RNA.

According to some embodiments of the invention, the silencing specificity of the non-coding RNA molecule is determined by measuring an RNA or protein level of the target RNA of interest.

According to some embodiments of the invention, the silencing specificity of the RNA silencing molecule is determined by measuring an RNA or protein level of the second target RNA.

According to some embodiments of the invention, the silencing specificity of the non-coding RNA molecule or the RNA silencing molecule is determined phenotypically.

According to some embodiments of the invention, the determined phenotypically is effected by determination of at least one phenotype selected from the group consisting of a cell size, a growth rate/inhibition, a cell shape, a cell membrane integrity, a tumor size, a tumor shape, a tumor vascularization, a pigmentation of an organism, a size of an organism, a crop yield, metabolic profile, a fruit trait, a biotic stress resistance, an abiotic stress resistance, an infection parameter, and an inflammation parameter.

According to some embodiments of the invention, the silencing specificity of the non-coding RNA molecule or the RNA silencing molecule is determined genotypically.

According to some embodiments of the invention, the phenotype is determined prior to a genotype.

According to some embodiments of the invention, the genotype is determined prior to a phenotype.

According to some embodiments of the invention, the non-coding RNA molecule or the RNA silencing molecule is processed from a precursor.

According to some embodiments of the invention, the non-coding RNA molecule or the RNA silencing molecule is processed into small RNA engaged with RNA-induced silencing complex (RISC).

According to some embodiments of the invention, the small RNA engaged with the RISC is selected from the group consisting of a small interfering RNA (siRNA), a short hairpin RNA (shRNA), a microRNA (miRNA), a Piwi-interacting RNA (piRNA), phased small interfering RNA (phasiRNA), trans-acting siRNA (tasiRNA), a small nuclear RNA (snRNA), a small nucleolar RNA (snoRNA), a long non-coding RNA (lncRNA), a ribosomal RNA (rRNA), transfer RNA (tRNA), a repeat-derived RNA, and an autonomous and non-autonomous transposable element RNA.

According to some embodiments of the invention, the small RNA engaged with the RISC is modified to preserve originality of structure and to be recognized by cellular RNAi factors.

According to some embodiments of the invention, the modifying the gene is affected by a modification selected from the group consisting of a deletion, an insertion, a point mutation and a combination thereof.

According to some embodiments of the invention, the modification is in a stem region of the non-coding RNA molecule or the RNA silencing molecule.

According to some embodiments of the invention, the modification is in a loop region of the non-coding RNA molecule or the RNA silencing molecule.

According to some embodiments of the invention, the modification is in a non-structured region of the non-coding RNA molecule or the RNA silencing molecule.

According to some embodiments of the invention, the modification is in a stem region and a loop region of the non-coding RNA molecule or the RNA silencing molecule.

According to some embodiments of the invention, the modification is in a stem region and a loop region and in non-structured region of the non-coding RNA molecule or the RNA silencing molecule.

According to some embodiments of the invention, the modification comprises a modification of at most 200 nucleotides.

According to some embodiments of the invention, the method does not comprise introducing into the eukaryotic cell donor oligonucleotides.

According to some embodiments of the invention, the method further comprises introducing into the eukaryotic cell donor oligonucleotides.

According to some embodiments of the invention, the DNA editing agent comprises at least one sgRNA.

According to some embodiments of the invention, the DNA editing agent elicits base editing.

According to some embodiments of the invention, the DNA or RNA editing agent does not comprise an endonuclease.

According to some embodiments of the invention, the DNA or RNA editing agent comprises an endonuclease.

According to some embodiments of the invention, the endonuclease comprises Cas9.

According to some embodiments of the invention, the endonuclease comprises a catalytically inactive endonuclease.

According to some embodiments of the invention, the DNA or RNA editing agent comprises an enzyme which is capable of epigenetic editing.

According to some embodiments of the invention, the enzyme which is capable of the epigenetic editing is selected from the group consisting of a DNA methyltransferase, a methylase, an acetyltransferase.

According to some embodiments of the invention, the enzyme which is capable of the epigenetic editing is selected from the group consisting of a DNA (cytosine-5)-methyltransferase 3A (DNMT3a), a Histone acetyltransferase p300, a Ten-eleven translocation methylcytosine dioxygenase 1 (TET1), Lysine (K)-specific demethylase 1A (LSD1) and Calcium and integrin binding protein 1 (CIB1).

According to some embodiments of the invention, the DNA editing agent comprises a DNA editing system selected from the group consisting of a meganuclease, a zinc finger nucleases (ZFN), a transcription-activator like effector nuclease (TALEN), CRISPR-endonuclease, dCRISPR-endonuclease, and a homing endonuclease.

According to some embodiments of the invention, the DNA editing agent is applied to the cell as DNA, RNA or RNP.

According to some embodiments of the invention, the DNA or RNA editing agent is linked to a reporter for monitoring expression in a eukaryotic cell.

According to some embodiments of the invention, the reporter is a fluorescent protein.

According to some embodiments of the invention, the target RNA of interest or the second target RNA is endogenous to the eukaryotic cell.

According to some embodiments of the invention, the target RNA of interest or the second target RNA is exogenous to the eukaryotic cell.

According to some embodiments of the invention, the target RNA of interest or the second target RNA is a transcript of a gene selected from the group consisting of a housekeeping gene, a dominant gene, a gene comprising a high copy number and a gene associated with cell apoptosis.

According to some embodiments of the invention, the gene associated with cell apoptosis is selected from the group consisting of BAX, PUMA and NOXA.

According to some embodiments of the invention, the eukaryotic cell is obtained from a eukaryotic organism selected from the group consisting of a plant, a mammal, an invertebrate, an insect, a nematode, a bird, a reptile, a fish, a crustacean, a fungi and an algae.

According to some embodiments of the invention, the eukaryotic cell is a plant cell.

According to some embodiments of the invention, the plant cell is a protoplast.

According to some embodiments of the invention, the breeding comprises crossing or selfing.

According to some embodiments of the invention, the eukaryotic cell is a non-human mammalian cell.

According to some embodiments of the invention, the eukaryotic cell is a human cell.

According to some embodiments of the invention, the eukaryotic cell is a totipotent stem cell.

According to some embodiments of the invention, the disease is selected from the group consisting of an infectious disease, a monogenic recessive disorder, an autoimmune disease and a cancerous disease.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of an embodiment computational pipeline to generate Genome Editing induced Gene Silencing (GEiGS) templates. The computational GEiGS pipeline applies biological metadata and enables an automatic generation of GEiGS DNA templates that are used to minimally edit miRNA genes, leading to a new gain of function, i.e. redirection of their silencing capacity to target sequence of interest.

FIG. 2 is an embodiment flowchart of GEiGS replacement of miRNA with siRNA targeting Green Fluorescent Protein (GFP), generating silencing of the stably expressed GFP gene in human cell lines.

FIGS. 3A-B are photographs illustrating knock down of GFP expression levels in human cells. Control cells (FIG. 3A) stably express GFP at high levels as compared to cells stably expressing siGFP in which GFP expression is silenced (FIG. 3B).

FIG. 4 is an embodiment flowchart of GEiGS cells stably expressing siGFP. All positive transfection events are red fluorescent proteins (RFP)+GFP. However, since GEiGS cells stably express siGFP, positive transfected cells show only red fluorescent expression.

FIG. 5 is an embodiment flowchart of GEiGS cells stably expressing siRNA targeting p53. All positive transfection events are GFP+ and evade chemotherapy or the hDM2 inhibitor Nutlin3-induced cell death.

FIG. 6 is an embodiment flowchart of GEiGS cells stably expressing siRNA targeting pro-apoptotic genes in human cancer cell line U2OS. All positive transfection events are RFP+ and evade chemotherapy-induced cell death.

FIG. 7 is an embodiment flowchart of GEiGS cells generated resistant to lentivirus infection (GFP is used as the virus marker gene or as the exogenous gene).

FIG. 8 is an embodiment flowchart of GEiGS cells generated that are resistant to virus infection (i.e. immunization of cells towards an exogenous viral gene).

FIG. 9 is an embodiment drawing illustrating the main stages required to design a RNA silencing molecule and with minimally edited miRNA gene bases.

FIG. 10 is a graph illustrating the diverse non-coding RNA types that are actively engaged in RNA interference (RNAi). The list provides non-coding RNA types that are both Dicer substrates (proven to be bound by Dicer) and are processed into small silencing RNA (their small RNAs are proven to be bound by Argonaute proteins) (axis y). Each type has multiple slightly different subtypes (axis x).

FIGS. 11A-E is an embodiment example of human non-coding RNAs that show the non-coding RNA precursor and its derived Ago-bound small RNAs. Shown are the AGO2- and AGO3-bound small RNAs mapped to Dicer-bound non-coding RNAs precursors. (FIG. 11A) shows the let7 miRNA and its primary (marked in blue line) and secondary mature miRNA sites (represented by gray bars). (FIGS. 11B-E) show examples of other biotypes where the small RNA mapping shows a signature analog to the one found in miRNAs.

FIGS. 12A-E are embodiment examples of GEiGS oligo designs. The selections of non-coding RNA precursors that give rise to mature small RNA molecules are highlighted in green. Sequence differences between the GEiGS oligos and the wild type sequence are highlighted in red. (FIG. 12A) Embodiment examples of GEiGS oligo designs in which the GEiGS precursors preserve identical secondary structure as the wild-type (wt) non-coding RNA. Design based on the Human miRNA-100. From left to right: wild-type miRNA, GEiGS design with matching structure and minimal sequence changes, and GEiGS design with matching structure and maximal sequence changes. Of note, the GEiGS designs were based on 21 nt siRNAs targeting Human heparin-binding vascular endothelial growth factor (VEGF); (FIG. 12B) Embodiment examples of GEiGS oligo designs in which the GEiGS precursors do not preserve the secondary structure of the wt non-coding RNA. Design based on the Human miRNA-100. From left to right: wild-type miRNA, GEiGS design with non-matching structure and minimal sequence changes, and GEiGS design with non-matching structure and maximal sequence changes. Of note, the GEiGS designs were based on 21 nt siRNAs targeting Human heparin-binding vascular endothelial growth factor (VEGF); (FIG. 12C) Embodiment examples of GEiGS oligo designs in which the GEiGS precursors preserve identical secondary structure as the wt non-coding RNA. Design based on the CID_001033 tRNA. From left to right: wild-type tRNA, GEiGS design with matching structure and minimal sequence changes, and GEiGS design with matching structure and maximal sequence changes. Of note, the GEiGS designs were based on 21 nt siRNAs targeting the bcr/abl e8a2 fusion protein gene; (FIG. 12D) Embodiment examples of GEiGS oligo designs in which the GEiGS precursors do not preserve the secondary structure of the wt non-coding RNA. Design based on the CID_001033 tRNA. From left to right, wild-type tRNA, GEiGS design with non-matching structure and minimal sequence changes, and GEiGS design with non-matching structure and maximal sequence changes. The GEiGS designs were based on 21 nt siRNAS targeting the bcr/abl e8a2 fusion protein gene; (FIG. 12E) Embodiment examples of GEiGS oligo designs in which the precursor structure does not play a role in the biogenesis, hence, it is not required to be maintained. Design based on the Brassica rapa bnTAS3B tasiRNA. From left to right: wild-type tasiRNA, GEiGS design with minimal sequence changes, and GEiGS design with maximal sequence changes. Of note, the circular structure is not inherent to the molecule and was applied for convenience; tasiRNA biogenesis, unlike miRNAs and tRNAs, does not rely on the precursor secondary structure (as discussed in detail in Borges and Martienssen (2015) Nature Reviews Molecular Cell Biology|AOP, published online 4 Nov. 2015; doi:10.1038/nrm4085). Below the full molecules there is a detail of the section containing modifications. The GEiGS designs were based on 21 nt siRNAS targeting the bcr/abl e8a2 fusion protein gene.

FIG. 13 illustrates PDS3 Phenotype/Genotype: bleached phenotype plants were selected and genotyped through internal amplicon PCR followed by restriction digest analysis with BtsαI (NEB) in order to verify donor presence vs. wild type sequence. Lane 1: Treated plants with NO DONOR, restricted, Lanes 2-4: PDS3 treated plants containing DONOR restricted, Lane 5: Positive plasmid DONOR control unrestricted, Lane 6: Water no template control, Lane 7: Positive Plasmid DONOR restricted, Lane 8: Plants bombarded with negative DONOR restricted, Lane 9: Untreated control plants restricted. Subsequent external PCR amplification of the amplicon was processed and sequenced in order to validate the insertion.

FIG. 14 illustrates ADH1 Phenotype/Genotype: Plants were selected through Allyl alcohol resistance and genotyped through internal amplicon PCR followed by BccI (NEB) restriction digest in order to verify donor presence. Lane 1: Allyl alcohol sensitive control plant restricted, Lane 2-4: Allyl alcohol resistant plants containing DONOR restricted, Lane5: Positive plasmid DONOR control unrestricted, Lane 6: no template control, Lane7: Positive Plasmid DONOR restricted, Lane 8: Plant bombarded with non-specific DONOR restricted, Lane 9: Non Allyl alcohol treated control restricted.

FIG. 15 is a graph illustrating gene expression analysis in miR-173 modified plant targeting AtPDS3 transcript. Analysis of AtPDS3 expression was carried out through qRT-PCR, in regenerating bombarded plants with GEiGS #4 and SWAP3 compared to plants bombarded with GEiGS #5 and SWAP1 and 2 (GFP). Of note, a reduction of 82% in gene expression level, on the average, was observed, when miR-173 was modified to target AtPDS3, compared to control plants (Error bars present SD; p-value <0.01 calculated on Ct values).

FIG. 16 is a graph illustrating gene expression analysis in miR-390 modified plant targeting AtPDS3 transcript. Analysis of AtADH1 expression was carried out through qRT-PCR, in regenerating bombarded plants with GEiGS #1 and SWAP11, compared to plants bombarded with GEiGS #5 and SWAP1 and 2 (GFP). Of note, a reduction of 82% in gene expression level, on the average, was observed, when miR-390 was modified to target AtADH1, compared to control plants (Error bars represent SD; p-value <0.01 calculated on Ct values).

### DESCRIPTION OF SPECIFIC EMBODIMENTS OF THE INVENTION

The present invention, in some embodiments thereof, relates to modifying genes that encode or are processed into non-coding RNA molecules, including RNA silencing molecules and, more particularly, but not exclusively, to the use of same for silencing endogenous or exogenous target RNA of interest in eukaryotic cells.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Two of the most powerful genetic therapeutic technologies developed thus far are gene therapy, which enables restoration of missing gene function by viral transgene expression, and RNAi, which mediates repression of defective genes by knockdown of the target mRNA. Recent advances in genome editing techniques have also made it possible to alter DNA sequences in living cells by editing one or more nucleotides in cells of human patients such as by genome editing (NHEJ and HR) following induction of site-specific double-strand breaks (DSBs) at desired locations in the genome.

While reducing the present invention to practice, the present inventors have devised a gene editing technology utilizing non-coding RNA molecules designed to target and interfere with any target gene of interest (endogenous or exogenous to the eukaryotic cell). The gene editing technology described herein does not necessitate the classical molecular genetic and transgenic tools comprising expression cassettes that have a promoter, terminator, selection marker. Moreover, the gene editing technology of some embodiments of the invention comprises genome editing of a non-coding RNA molecule (e.g. endogenous) yet it is stable and heritable.

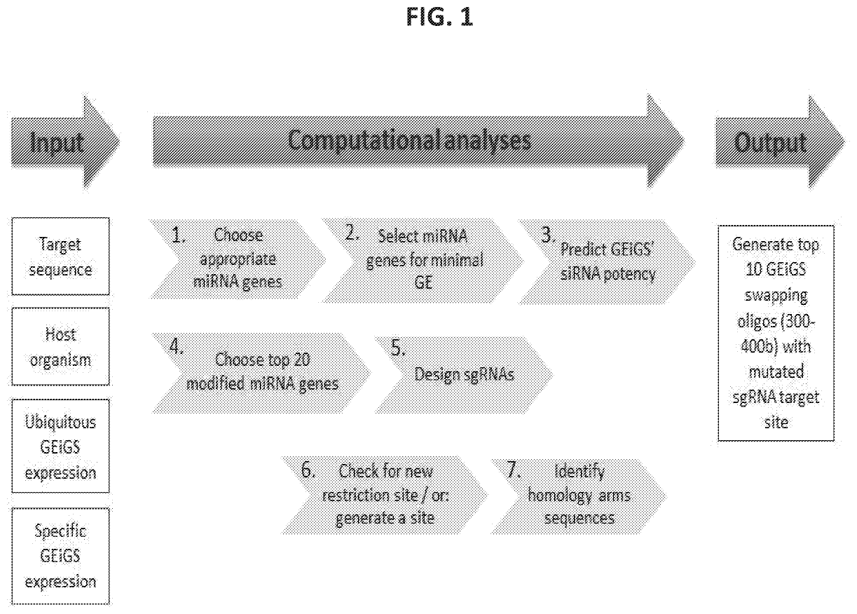

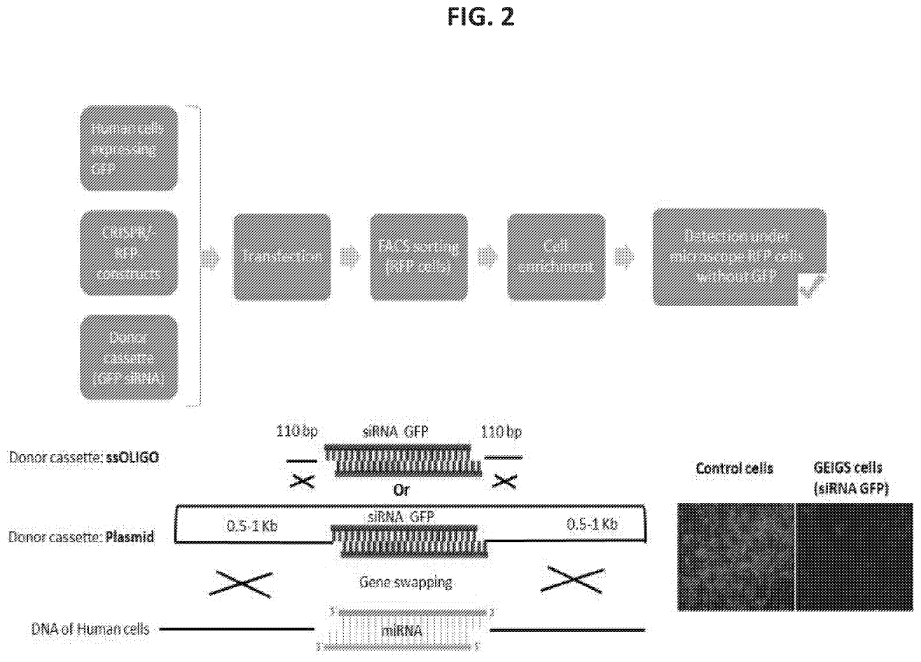

As is shown herein below and in the Examples section which follows, the present inventors have designed a Genome Editing induced Gene Silencing (GEiGS) platform capable of utilizing a eukaryotic cell's endogenous non-coding RNA molecules including e.g. RNA silencing molecules (e.g. siRNA, miRNA, piRNA, tasiRNA, tRNA, rRNA, antisense RNA, etc.) and modifying them to target any RNA target of interest (see exemplary flowchart in FIG. 2). Using GEiGS, the present method enables screening of potential non-coding RNA molecules, editing a few nucleotides in these endogenous RNA molecules, and thereby redirecting their activity and/or specificity to effectively and specifically target any RNA of interest including, for instance, endogenous RNA coding for mutated proteins (e.g. oncogenes in cancers) or exogenous RNA encoded by pathogens (see exemplary flowchart in FIG. 1). The present method is specifically suitable for downregulation of gene expression wherein the gene is critical for eukaryotic cell viability, has a high copy number (e.g. ploidy) or is a dominant gene. The present method is suitable for genetic modifications utilizing DNA and RNA editing methods, including base editing, of various non-coding RNA molecules including situations in which the gene encoding or processed into the non-coding RNA molecule is positioned in a coding gene. Taken together, GEiGS can be utilized as a novel technology for modulation of endogenous gene expression and also to protect organisms against different biotic and abiotic stresses such as e.g. cancer, viruses, insects, fungi, nematodes, heat, drought, starvation etc.

Thus, according to one aspect of the present invention there is provided a method of modifying a gene encoding or processed into a non-coding RNA molecule having no RNA silencing activity in a eukaryotic cell, wherein the gene encoding or processed into the non-coding RNA molecule is positioned in a coding gene, the method comprising introducing into the eukaryotic cell a DNA editing agent conferring a silencing specificity of the non-coding RNA molecule towards a target RNA of interest, thereby modifying the gene encoding or processed into the non-coding RNA molecule.

According to another aspect of the invention there is provided a method of modifying a gene encoding or processed into a RNA silencing molecule to a target RNA in a eukaryotic cell, wherein the gene encoding or processed into the non-coding RNA molecule is positioned in a coding gene, the method comprising introducing into the eukaryotic cell a DNA editing agent which redirects a silencing specificity of the RNA silencing molecule towards a second target RNA, the target RNA and the second target RNA being distinct, thereby modifying the gene encoding or processed into the RNA silencing molecule.

According to another aspect of the invention there is provided a method of modifying a gene encoding or processed into a non-coding RNA molecule having no RNA silencing activity in a eukaryotic cell, the method comprising introducing into the eukaryotic cell a DNA or RNA editing agent conferring a silencing specificity of the non-coding RNA molecule towards a target RNA of interest, wherein the DNA or RNA editing agent elicits base editing, thereby modifying the gene encoding or processed into the non-coding RNA molecule.

According to another aspect of the invention there is provided a method of modifying a gene encoding or processed into a RNA silencing molecule to a target RNA in a eukaryotic cell, the method comprising introducing into the eukaryotic cell a DNA or RNA editing agent which redirects a silencing specificity of the RNA silencing molecule towards a second target RNA, the target RNA and the second target RNA being distinct, and wherein the DNA or RNA editing agent elicits base editing, thereby modifying the gene encoding or processed into the RNA silencing molecule.

According to another aspect of the invention there is provided a method of modifying a gene encoding or processed into a non-coding RNA molecule having no RNA silencing activity in a eukaryotic cell, the method comprising introducing into the eukaryotic cell a DNA editing agent conferring a silencing specificity of the non-coding RNA molecule towards a target RNA of interest, wherein the target RNA of interest is a transcript of a gene selected from the group consisting of a housekeeping gene, a dominant gene, a gene comprising a high copy number, and a gene associated with cell apoptosis, thereby modifying the gene encoding or processed into the non-coding RNA molecule.

According to another aspect of the invention there is provided a method of modifying a gene encoding or processed into a RNA silencing molecule to a target RNA in a eukaryotic cell, the method comprising introducing into the eukaryotic cell a DNA editing agent which redirects a silencing specificity of the RNA silencing molecule towards a second target RNA, wherein the second target RNA is a transcript of a gene selected from the group consisting of a housekeeping gene, a dominant gene, a gene comprising a high copy number, and a gene associated with cell apoptosis, the target RNA and the second target RNA being distinct, thereby modifying the gene encoding or processed into the RNA silencing molecule.

The term “eukaryotic cell” as used herein refers to any cell of a eukaryotic organism. Eukaryotic organisms include single- and multi-cellular organisms. Single cell eukaryotic organisms include, but are not limited to, yeast, protozoans, slime molds and algae. Multi-cellular eukaryotic organisms include, but are not limited to, animals (e.g. mammals, insects, invertebrates, nematodes, birds, fish, reptiles and crustaceans), plants, fungi and algae (e.g. brown algae, red algae, green algae).

According to one embodiment, the cell is a plant cell.

According to a specific embodiment, the plant cell is a protoplast.

The protoplasts are derived from any plant tissue e.g., fruit, flowers, roots, leaves, embryos, embryonic cell suspension, calli or seedling tissue (as discussed below).

According to a specific embodiment, the plant cell is an embryogenic cell.

According to a specific embodiment, the plant cell is a somatic embryogenic cell.

According to one embodiment, the eukaryotic cell is not a cell of a plant.

According to a one embodiment, the eukaryotic cell is an animal cell.

According to a one embodiment, the eukaryotic cell is a cell of a vertebrate.

According to a one embodiment, the eukaryotic cell is a cell of an invertebrate.

According to a specific embodiment, the invertebrate cell is a cell of an insect, a snail, a clam, an octopus, a starfish, a sea-urchin, a jellyfish, and a worm.

According to a specific embodiment, the invertebrate cell is a cell of a crustacean. Exemplary crustaceans include, but are not limited to, shrimp, prawns, crabs, lobsters and crayfishes.

According to a specific embodiment, the invertebrate cell is a cell of a fish. Exemplary fish include, but are not limited to, Salmon, Tuna, Pollock, Catfish, Cod, Haddock, Prawns, Sea bass, Tilapia, Arctic char and Carp.

According to a one embodiment, the eukaryotic cell is a mammalian cell.

According to a specific embodiment, the mammalian cell is a cell of a non-human organism, such as but not limited to, a rodent, a rabbit, a pig, a goat, a ruminant (e.g. cattle, sheep, antelope, deer, and giraffe), a dog, a cat, a horse, and non-human primate.

According to a specific embodiment, the eukaryotic cell is a cell of human being.

According to one embodiment, the eukaryotic cell is a primary cell, a cell line, a somatic cell, a germ cell, a stem cell, an embryonic stem cell, an adult stem cell, a hematopoietic stem cell, a mesenchymal stem cell, an induced pluripotent stem cell (iPS), a gamete cell, a zygote cell, a blastocyst cell, an embryo, a fetus and/or a donor cell.

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells). Totipotent cells, such as embryonic cells within the first couple of cell divisions after fertilization are the only cells that can differentiate into embryonic and extra-embryonic cells and are able to develop into a viable human being. Preferably, the phrase “pluripotent stem cells” refers to cells which can differentiate into all three embryonic germ layers, i.e., ectoderm, endoderm and mesoderm or remaining in an undifferentiated state. The pluripotent stem cells include embryonic stem cells (ESCs) and induced pluripotent stem cells (iPS). The multipotent stem cells include adult stem cells and hematopoietic stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage.

It will be appreciated that commercially available stem cells can also be used according to some embodiments of the invention. Human ES cells can be purchased from the NIH human embryonic stem cells registry [www(dot)grants(dot)nih(dot) gov/stem_cells/registry/current(dot)html].

In addition, embryonic stem cells can be obtained from various species, including mouse (Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

“Induced pluripotent stem cells” (iPS; embryonic-like stem cells) refers to cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which reprogram the cell to acquire embryonic stem cells characteristics. According to some embodiments of the invention, the induced pluripotent stem cells are formed by inducing the expression of Oct-4, Sox2, Kfl4 and c-Myc in a somatic stem cell.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [such as described in Park et al. Reprogramming of human somatic cells to pluripotency with defined factors. Nature (2008) 451:141-146].

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

According to one embodiment, the stem cells utilized by some embodiments of the invention are bone marrow (BM)-derived stem cells including hematopoietic, stromal or mesenchymal stem cells [Dominici, M et al., (2001) J. Biol. Regul. Homeost. Agents. 15: 28-37]. BM-derived stem cells may be obtained from iliac crest, femora, tibiae, spine, rib or other medullar spaces.

Hematopoietic stem cells (HSCs), which may also be referred to as adult tissue stem cells, include stem cells obtained from blood or bone marrow tissue of an individual at any age or from cord blood of a newborn individual. Preferred stem cells according to this aspect of some embodiments of the invention are embryonic stem cells, preferably of a human or primate (e.g., monkey) origin.

Placental and umbilical cord blood stem cells may also be referred to as “young stem cells”.

Mesenchymal stem cells (MSCs), the formative pluripotent blast cells, give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the BM far exceeds their abundance in other tissues and as such isolation from BM is presently preferred.

Adult tissue stem cells can be isolated using various methods known in the art such as those disclosed by Alison, M. R. [J Pathol. (2003) 200(5): 547-50]. Fetal stem cells can be isolated using various methods known in the art such as those disclosed by Eventov-Friedman S, et al. [PLoS Med. (2006) 3: e215].

Hematopoietic stem cells can be isolated using various methods known in the arts such as those disclosed by “Handbook of Stem Cells” edit by Robert Lanze, Elsevier Academic Press, 2004, Chapter 54, pp 609-614, isolation and characterization of hematopoietic stem cells, by Gerald J Spangrude and William B Stayton.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

According to one embodiment, the eukaryotic cell is isolated from its natural environment (e.g. human body).

According to one embodiment, the eukaryotic cell is a healthy cell.

According to one embodiment, the eukaryotic cell is a diseased cell or a cell prone to a disease.

According to one embodiment, the eukaryotic cell is a cancer cell.

According to one embodiment, the eukaryotic cell is an immune cell (e.g. T cell, B cell, macrophage, NK cell, etc.).

According to one embodiment, the eukaryotic cell is a cell infected by a pathogen (e.g. by a bacterial, viral or fungal pathogen).

As used herein, the term “non-coding RNA molecule” refers to a RNA sequence that is not translated into an amino acid sequence and does not encode a protein.

According to one embodiment, the non-coding RNA molecule is typically subject to the RNA silencing processing mechanism or activity. However, also contemplated herein are a few changes in nucleotides (e.g. up to 24 nucleotides) which may elicit a processing mechanism that results in RNA interference or translation inhibition.

According to a specific embodiment, the non-coding RNA molecule is endogenous (naturally occurring, e.g. native) to the cell.

It will be appreciated that the non-coding RNA molecule can also be exogenous to the cell (i.e. externally added and which is not naturally occurring in the cell).

According to some embodiments, the non-coding RNA molecule comprises an intrinsic translational inhibition activity.

According to some embodiments, the non-coding RNA molecule comprises an intrinsic RNAi activity.

According to some embodiments, the non-coding RNA molecule does not comprise an intrinsic translational inhibition activity or an intrinsic RNAi activity (i.e. the non-coding RNA molecule does not have an RNA silencing activity).

According to an embodiment of the invention, the non-coding RNA molecule is specific to a target RNA (e.g., a natural target RNA) and does not cross inhibit or silence a second target RNA or target RNA of interest unless designed to do so (as discussed below) exhibiting 100% or less global homology to the target gene, e.g., less than 99%, less than 98%, 97%, 96%, 95%, 94%, 93%, 92%, 91%, 90%, 89%, 88%, 87%, 86%, 85%, 84%, 83%, 82%, 81% global homology to the target gene; as determined at the RNA or protein level by RT-PCR, Western blot, Immunohistochemistry and/or flow cytometry or any other detection methods.

According to one embodiment, the non-coding RNA molecule is a RNA silencing or RNA interference (RNAi) molecule.

The term “RNA silencing” or RNAi refers to a cellular regulatory mechanism in which non-coding RNA molecules (the “RNA silencing molecule” or “RNAi molecule”) mediate, in a sequence specific manner, co- or post-transcriptional inhibition of gene expression or translation.

According to one embodiment, the RNA silencing molecule is capable of mediating RNA repression during transcription (co-transcriptional gene silencing).

According to a specific embodiment, co-transcriptional gene silencing includes epigenetic silencing (e.g. chromatic state that prevents gene expression).

According to one embodiment, the RNA silencing molecule is capable of mediating RNA repression after transcription (post-transcriptional gene silencing).

Post-transcriptional genes silencing (PTGS) typically refers to the process of degradation or cleavage of messenger RNA (mRNA) molecules which decrease their activity by preventing translation. For example, and as discussed in detail below, a guide strand of a RNA silencing molecule pairs with a complementary sequence in a mRNA molecule and induces cleavage by e.g. Argonaute 2 (Ago2).

Co-transcriptional gene silencing typically refers to inactivation of gene activity (i.e. transcription repression) and typically occurs in the cell nucleus. Such gene activity repression is mediated by epigenetic-related factors, such as e.g. methyl-transferases, that methylate target DNA and histones. Thus, in co-transcriptional gene silencing, the association of a small RNA with a target RNA (small RNA-transcript interaction) destabilizes the target nascent transcript and recruits DNA- and histone-modifying enzymes (i.e. epigenetic factors) that induce chromatin remodeling into a structure that repress gene activity and transcription. Also, in co-transcriptional gene silencing, chromatin-associated long non-coding RNA scaffolds may recruit chromatin-modifying complexes independently of small RNAs. These co-transcriptional silencing mechanisms form RNA surveillance systems that detect and silence inappropriate transcription events, and provide a memory of these events via self-reinforcing epigenetic loops [as described in D. Hoch and D. Moazed, RNA-mediated epigenetic regulation of gene expression, Nat Rev Genet. (2015) 16(2): 71-84].

According to an embodiment of the invention, the RNAi biogenesis/processing machinery generates the RNA silencing molecule.

According to an embodiment of the invention, the RNAi biogenesis/processing machinery generates the RNA silencing molecule, but no specific target has been identified.

According to one embodiment, the non-coding RNA molecule is a capable of inducing RNA interference (RNAi).

According to one embodiment, the non-coding RNA molecule or the RNA silencing molecule is processed from a precursor.

According to one embodiment, the non-coding RNA molecule or RNA silencing molecule is processed from a single stranded RNA (ssRNA) precursor.

According to one embodiment, the non-coding RNA molecule or the RNA silencing molecule is processed from a duplex-structured single-stranded RNA precursor.

According to one embodiment, the non-coding RNA molecule or RNA silencing molecule is processed from a dsRNA precursor (e.g. comprising perfect and imperfect base pairing).

According to one embodiment, the dsRNA can be derived from two different complementary RNAs, or from a single RNA that folds on itself to form dsRNA.

According to one embodiment, the non-coding RNA molecule or the RNA silencing molecule is processed from a non-structured RNA precursor.

According to one embodiment, the non-coding RNA molecule or the RNA silencing molecule is processed from a protein-coding RNA precursor.

According to one embodiment, the non-coding RNA molecule or the RNA silencing molecule is processed from a non-coding RNA precursor.

According to one embodiment, the dsRNA can be derived from two different complementary RNAs, or from a single RNA that folds on itself to form dsRNA.

The terms “processing” or “processability” refer to the biogenesis by which RNA molecules are cleaved into small RNA form capable of engaging with RNA-induced silencing complex (RISC). Exemplary processing mechanisms include e.g., Dicer and Argonaute, as further discussed below. For example, pre-miRNA is processed into a mature miRNA by Dicer.

As used herein, the term “small RNA form” or “small RNAs” or “small RNA molecule” refers to the mature small RNA being capable of hybridizing with a target RNA (or fragment thereof).

According to one embodiment, the small RNA form has a silencing activity.

According to one embodiment, the small RNAs comprise no more than 250 nucleotides in length, e.g. comprise 15-250, 15-200, 15-150, 15-100, 15-50, 15-40, 15-30, 15-25, 15-20, 20-30, 20-25, 30-100, 30-80, 30-60, 30-50, 30-40, 30-35, 50-150, 50-100, 50-80, 50-70, 50-60, 100-250, 100-200, 100-150, 150-250, 150-200 nucleotides.

According to a specific embodiment, the small RNA molecules comprise 20-50 nucleotides.

According to a specific embodiment, the small RNA molecules comprise 20-30 nucleotides.

According to a specific embodiment, the small RNA molecules comprise 21-29 nucleotides.

According to a specific embodiment, the small RNA molecules comprise 21-23 nucleotides.

According to a specific embodiment, the small RNA molecules comprise 21 nucleotides.

According to a specific embodiment, the small RNA molecules comprise 22 nucleotides.

According to a specific embodiment, the small RNA molecules comprise 23 nucleotides.

According to a specific embodiment, the small RNA molecules comprise 24 nucleotides.

Typically, processability depends on a structure of a RNA molecule, also referred to herein as originality of structure, i.e. the secondary RNA structure (i.e. base pairing profile). The originality of structure is important for correct and efficient processing of the RNA molecule into small RNAs (such as siRNA or miRNA) that is structure- and not purely sequence-dependent.

According to one embodiment, the cellular RNAi processing machinery, i.e. cellular RNAi processing and executing factors, process the non-coding RNA molecules into small RNAs.

According to one embodiment, the cellular RNAi processing machinery comprises ribonucleases, including but not limited to, the DICER protein family (e.g. DCR1 and DCR2), DICER-LIKE protein family (e.g. DCL1, DCL2, DCL3, DCL4), ARGONAUTE protein family (e.g. AGO1, AGO2, AGO3, AGO4), tRNA cleavage enzymes (e.g. RNY1, ANGIOGENIN, RNase P, RNase P-like, SLFN3, ELAC1 and ELAC2), and Piwi-interacting RNA (piRNA) related proteins (e.g. AGO3, AUBERGINE, HIWI, HIWI2, HIWI3, PIWI, ALG1 and ALG2).

Following is a detailed description of non-coding RNA molecules which comprise an intrinsic RNAi activity (e.g. are RNA silencing molecules) that can be used according to specific embodiments of the present invention.

Perfect and imperfect based paired RNA (i.e. double stranded RNA; dsRNA), siRNA and shRNA—The presence of long dsRNAs in cells stimulates the activity of a ribonuclease III enzyme referred to as dicer. Dicer (also known as endoribonuclease Dicer or helicase with RNase motif) is an enzyme that in plants is typically referred to as Dicer-like (DCL) protein. Different plants have different numbers of DCL genes, thus for example, Arabidopsis genome typically has four DCL genes, rice has eight DCL genes, and maize genome has five DCL genes. Dicer is involved in the processing of the dsRNA into short pieces of dsRNA known as short interfering RNAs (siRNAs). siRNAs derived from dicer activity are typically about 21 to about 23 nucleotides in length and comprise about 19 base pair duplexes with two 3′ nucleotides overhangs.

Accordingly, some embodiments of the invention contemplate modifying a gene encoding a dsRNA to redirect a silencing specificity (including silencing activity) towards a second target RNA (i.e. RNA of interest).

According to one embodiment dsRNA precursors longer than 21 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position, but not the composition, of the 3′-overhang influences potency of a siRNA and asymmetric duplexes having a 3′-overhang on the antisense strand are generally more potent than those with the 3′-overhang on the sense strand (Rose et al., 2005).

The strands of a double-stranded interfering RNA (e.g., a siRNA) may be connected to form a hairpin or stem-loop structure (e.g., a shRNA). Thus, as mentioned, the RNA silencing molecule of some embodiments of the invention may also be a short hairpin RNA (shRNA).

The term short hairpin RNA, “shRNA”, as used herein, refers to a RNA molecule having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

The RNA silencing molecule of some embodiments of the invention need not be limited to those molecules containing only RNA, but further encompasses chemically-modified nucleotides and non-nucleotides.

Various types of siRNAs are contemplated by the present invention, including trans-acting siRNAs (Ta-siRNAs or TasiRNA), repeat-associated siRNAs (Ra-siRNAs) and natural-antisense transcript-derived siRNAs (Nat-siRNAs).

According to one embodiment, silencing RNA includes “piRNA” which is a class of Piwi-interacting RNAs of about 26 and 31 nucleotides in length. piRNAs typically form RNA-protein complexes through interactions with Piwi proteins, i.e. antisense piRNAs are typically loaded into Piwi proteins (e.g. Piwi, Ago3 and Aubergine (Aub)).

miRNA—According to another embodiment the RNA silencing molecule may be a miRNA.

The term “microRNA”, “miRNA”, and “miR” are synonymous and refer to a collection of non-coding single-stranded RNA molecules of about 19-24 nucleotides in length, which regulate gene expression. miRNAs are found in a wide range of organisms (e.g. insects, mammals, plants, nematodes) and have been shown to play a role in development, homeostasis, and disease etiology.

Initially the pre-miRNA is present as a long non-perfect double-stranded stem loop RNA that is further processed by Dicer into a siRNA-like duplex, comprising the mature guide strand (miRNA) and a similar-sized fragment known as the passenger strand (miRNA*). The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-8 of the miRNA (referred as “seed sequence”).

A number of studies have looked at the base-pairing requirement between miRNA and its mRNA target for achieving efficient inhibition of translation (reviewed by Bartel 2004, Cell 116-281). Computational studies, analyzing miRNA binding on whole genomes have suggested a specific role for bases 2-8 at the 5′ of the miRNA (also referred to as “seed sequence”) in target binding but the role of the first nucleotide, found usually to be “A” was also recognized (Lewis et al. 2005 Cell 120-15). Similarly, nucleotides 1-7 or 2-8 were used to identify and validate targets by Krek et al. (2005, Nat Genet 37-495). The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

According to one embodiment, miRNAs can be processed independently of Dicer, e.g. by Argonaute 2.

It will be appreciated that the pre-miRNA sequence may comprise from 45-90, 60-80 or 60-70 nucleotides while the pri-miRNA sequence may comprise from 45-30,000, 50-25,000, 100-20,000, 1,000-1,500 or 80-100 nucleotides.

According to one embodiment, the miRNA comprises miR-150 (e.g. human miR-150, e.g. as set forth in SEQ ID NO: 13).

According to one embodiment, the miRNA comprises miR-210 (e.g. human miR-210, e.g. as set forth in SEQ ID NO: 14).

According to one embodiment, the miRNA comprises Let-7 (e.g. human Let-7, e.g. as set forth in SEQ ID NO: 15).

According to one embodiment, the miRNA comprises miR-184 (e.g. human miR-184, e.g. as set forth in SEQ ID NO: 16).

According to one embodiment, the miRNA comprises miR-204 (e.g. human miR-204, e.g. as set forth in SEQ ID NO: 17).

According to one embodiment, the miRNA comprises miR-25 (e.g. human miR-25, e.g. as set forth in SEQ ID NO: 18).

According to one embodiment, the miRNA comprises miR-34 (e.g. human miR-34a/b/c, e.g. as set forth in SEQ ID NOs: 19-21, respectively).

Antisense—Antisense is a single stranded RNA designed to prevent or inhibit expression of a gene by specifically hybridizing to its mRNA. Downregulation of a target RNA can be effected using an antisense polynucleotide capable of specifically hybridizing with an mRNA transcript encoding the target RNA.

Transposable Element RNA

Transposable genetic elements (TEs) comprise a vast array of DNA sequences, all having the ability to move to new sites in genomes either directly by a cut-and-paste mechanism (transposons) or indirectly through an RNA intermediate (retrotransposons). TEs are divided into autonomous and non-autonomous classes depending on whether they have ORFs that encode proteins required for transposition. RNA-mediated gene silencing is one of the mechanisms in which the genome control TEs activity and deleterious effects derived from genome genetic and epigenetic instability.

As mentioned, the non-coding RNA molecule may not comprise a canonical (intrinsic) RNAi activity (e.g. is not a canonical RNA silencing molecule, or its target has not been identified). Such non-coding RNA molecules include the following:

According to one embodiment, the RNA silencing molecule is a transfer RNA (tRNA). The term “tRNA” refers to a RNA molecule that serves as the physical link between nucleotide sequence of nucleic acids and the amino acid sequence of proteins, formerly referred to as soluble RNA or sRNA. tRNA is typically about 76 to 90 nucleotides in length.

According to one embodiment, the RNA silencing molecule is a ribosomal RNA (rRNA). The term “rRNA” refers to the RNA component of the ribosome i.e. of either the small ribosomal subunit or the large ribosomal subunit.

According to one embodiment, the RNA silencing molecule is a small nuclear RNA (snRNA or U-RNA). The terms “sRNA” or “U-RNA” refer to the small RNA molecules found within the splicing speckles and Cajal bodies of the cell nucleus in eukaryotic cells. snRNA is typically about 150 nucleotides in length.

According to one embodiment, the RNA silencing molecule is a small nucleolar RNA (snoRNA). The term “snoRNA” refers to the class of small RNA molecules that primarily guide chemical modifications of other RNAs, e.g. rRNAs, tRNAs and snRNAs. snoRNA is typically classified into one of two classes: the C/D box snoRNAs are typically about 70-120 nucleotides in length and are associated with methylation, and the H/ACA box snoRNAs are typically about 100-200 nucleotides in length and are associated with pseudouridylation.

Similar to snoRNAs are the scaRNAs (i.e. Small Cajal body RNA genes) which perform a similar role in RNA maturation to snoRNAs, but their targets are spliceosomal snRNAs and they perform site-specific modifications of spliceosomal snRNA precursors (in the Cajal bodies of the nucleus).

According to one embodiment, the RNA silencing molecule is an extracellular RNA (exRNA). The term “exRNA” refers to RNA species present outside of the cells from which they were transcribed (e.g. exosomal RNA).

According to one embodiment, the RNA silencing molecule is a long non-coding RNA (lncRNA). The term “lncRNA” or “long ncRNA” refers to non-protein coding transcripts typically longer than 200 nucleotides.

According to a specific embodiment, non-limiting examples of non-coding RNA molecules include, but are not limited to, microRNA (miRNA), piwi-interacting RNA (piRNA), short interfering RNA (siRNA), short-hairpin RNA (shRNA), phased small interfering RNA (phasiRNA), trans-acting siRNA (tasiRNA), small nuclear RNA (snRNA or URNA), transposable element RNA (e.g. autonomous and non-autonomous transposable RNA), transfer RNA (tRNA), small nucleolar RNA (snoRNA), Small Cajal body RNA (scaRNA), ribosomal RNA (rRNA), extracellular RNA (exRNA), repeat-derived RNA, and long non-coding RNA (lncRNA).

According to a specific embodiment, non-limiting examples of RNAi molecules include, but are not limited to, small interfering RNA (siRNA), short hairpin RNA (shRNA), microRNA (miRNA), Piwi-interacting RNA (piRNA), phased small interfering RNA (phasiRNA), and trans-acting siRNA (tasiRNA).

According to one embodiment, the gene encoding or processed into a non-coding RNA molecule or into a RNA silencing molecule is positioned in a non-coding gene. Exemplary non-coding parts of the genome include, but are not limited to, genes of non-coding RNAs, enhancers and locus control regions, insulators, S/MAR sequences, non-coding pseudogenes, non-autonomous transposons and retrotransposons, and non-coding simple repeats of centromeric and telomeric regions of chromosomes.

According to one embodiment, the gene encoding or processed into a non-coding RNA molecule or into a RNA silencing molecule is positioned in a non-coding gene that is ubiquitously expressed.

According to one embodiment, the gene encoding or processed into a non-coding RNA molecule or into a RNA silencing molecule is positioned in a non-coding gene that is expressed in a tissue-specific manner.

According to one embodiment, the gene encoding or processed into a non-coding RNA molecule or into a RNA silencing molecule is positioned in a non-coding gene that is expressed in an inducible manner.

According to one embodiment, the gene encoding or processed into a non-coding RNA molecule or into a RNA silencing molecule is positioned in a non-coding gene that is developmentally regulated.

According to one embodiment, the gene encoding or processed into a non-coding RNA molecule or into a RNA silencing molecule is positioned between genes, i.e. intergenic region.

According to one embodiment, the gene encoding or processed into a non-coding RNA molecule or into a RNA silencing molecule is positioned in a coding gene (e.g. protein-coding gene).

According to one embodiment, the gene encoding or processed into a non-coding RNA molecule or into a RNA silencing molecule is positioned within an exon of a coding gene (e.g. protein-coding gene).

According to one embodiment, the gene encoding or processed into a non-coding RNA molecule or into a RNA silencing molecule is positioned within an exon encoding an untranslated region (UTR) of a coding gene (e.g. protein-coding gene).

According to one embodiment, the gene encoding or processed into a non-coding RNA molecule or into a RNA silencing molecule is positioned within a translated exon of a coding gene (e.g. protein-coding gene).

According to one embodiment, the gene encoding or processed into a non-coding RNA molecule or into a RNA silencing molecule is positioned within an intron of a coding gene (e.g. protein-coding gene).

According to one embodiment, the gene encoding or processed into a non-coding RNA molecule or into a RNA silencing molecule is positioned within a coding gene that is ubiquitously expressed.

According to one embodiment, the gene encoding or processed into a non-coding RNA molecule or into a RNA silencing molecule is positioned within a coding gene that is expressed in a tissue-specific manner.

According to one embodiment, the gene encoding or processed into a non-coding RNA molecule or into a RNA silencing molecule is positioned within coding gene that is expressed in an inducible manner.

According to one embodiment, the gene encoding or processed into a non-coding RNA molecule or into a RNA silencing molecule is positioned within coding gene that is developmentally regulated.

As mentioned above, the methods of some embodiments of the invention are utilized to redirect a silencing activity and/or specificity of the non-coding RNA molecule (or to generate a silencing activity and/or specificity if the non-coding RNA molecule does not have an intrinsic capability to silence a RNA molecule) towards a second target RNA or towards a target RNA of interest.

According to one embodiment, the target RNA and the second target RNA are distinct.

According to one embodiment, the method of modifying a gene encoding or processed into a RNA silencing molecule to a target RNA in a eukaryotic cell, comprises introducing into the eukaryotic cell a DNA editing agent which redirects a silencing activity and/or specificity of the RNA silencing molecule towards a second target RNA, the target RNA and the second target RNA being distinct, thereby modifying the gene encoding the RNA silencing molecule.

As used herein, the term “redirects a silencing specificity” refers to reprogramming the original specificity of the non-coding RNA (e.g. RNA silencing molecule) towards a non-natural target of the non-coding RNA (e.g. RNA silencing molecule). Accordingly, the original specificity of the non-coding RNA is destroyed (i.e. loss of function) and the new specificity is towards a RNA target distinct of the natural target (i.e. RNA of interest), i.e., gain of function. It will be appreciated that only gain of function occurs in cases that the non-coding RNA has no silencing activity.

As used herein, the term “target RNA” refers to a RNA sequence naturally bound by a non-coding RNA molecule. Thus, the target RNA is considered by the skilled artisan as a substrate for the non-coding RNA.

As used herein, the term “second target RNA” refers to a RNA sequence (coding or non-coding) not naturally bound by a non-coding RNA molecule. Thus, the second target RNA is not a natural substrate of the non-coding RNA.

As used herein, the term “target RNA of interest” refers to a RNA sequence (coding or non-coding) to be silenced by the designed non-coding RNA molecule.

As used herein, the phrase “silencing a target gene” refer to the absence or observable reduction in the level of protein and/or mRNA product from the target gene. Thus, silencing of a target gene can be by 5%, 10%, 20%, 30%, 40%, 50%, 60%, 70%, 80%, 90%, 95% or 100% as compared to a target gene not targeted by the designed non-coding RNA molecule of the invention.

The consequences of silencing can be confirmed by examination of the outward properties of a eukaryotic cell or organism, or by biochemical techniques (as discussed below).

It will be appreciated that the designed non-coding RNA molecule of some embodiments of the invention can have some off-target specificity effect/s provided that it does not affect the growth, differentiation or function of the eukaryotic cell or organism.

According to one embodiment, the second target RNA or target RNA of interest is endogenous to the eukaryotic cell.

According to one embodiment, the second target RNA or target RNA of interest is a transcript of a housekeeping gene.

According to one embodiment, the second target RNA or target RNA of interest is a transcript of a dominant gene.

According to one embodiment, the second target RNA or target RNA of interest is a transcript of a gene comprising a high copy number.

According to one embodiment, the second target RNA or target RNA of interest is a transcript of a gene associated with cell apoptosis. Exemplary genes associated with cell apoptosis include, but are not limited to, proapoptotic Bcl2 family members e.g. p53 upregulated modulator of apoptosis (PUMA), NOXA, and BAX. Additional genes associated with cell apoptosis are described in Wang et al. Comput Math Methods Med. (2015) 2015:715639, doi: 10.1155/2015/715639, incorporated herein by reference in its entirety.

Exemplary endogenous second target RNA or target RNA of interest include, but are not limited to, a product of a gene associated with cancer and/or apoptosis. Exemplary target genes associated with cancer include, but are not limited to, p53, BAX, PUMA, NOXA and FAS genes as discussed in detail herein below.

According to one embodiment, the second target RNA or target RNA of interest is exogenous to the eukaryotic cell (also referred to herein as heterologous). In such a case, the second target RNA or target RNA of interest is a product of a gene that is not naturally part of the eukaryotic cell genome (i.e. which expresses the non-coding RNA). Exemplary exogenous target RNAs include, but are not limited to, products of a gene associated with an infectious disease, such as a gene of a pathogen (e.g. an insect, a virus, a bacteria, a fungi, a nematode), as further discussed herein below. An exogenous target RNA (coding or non-coding) may comprise a nucleic acid sequence which shares sequence identity with an endogenous RNA sequence (e.g. may be partially homologous to an endogenous nucleic acid sequence) of the eukaryotic organism.

The specific binding of an endogenous non-coding RNA molecule with a target RNA can be determined by computational algorithms (such as BLAST) and verified by methods including e.g. Northern blot, In Situ hybridization, QuantiGene Plex Assay etc.

By use of the term “complementarity” or “complementary” is meant that the non-coding RNA molecule (or at least a portion of it that is present in the processed small RNA form, or at least one strand of a double-stranded polynucleotide or portion thereof, or a portion of a single strand polynucleotide) hybridizes under physiological conditions to the target RNA, or a fragment thereof, to effect regulation or function or suppression of the target gene. For example, in some embodiments, a non-coding RNA molecule has 100 percent sequence identity or at least about 30, 40, 45, 50, 55, 60, 65, 70, 75, 80, 83, 84, 85, 86, 87, 88, 89, 90, 91, 92, 93, 94, 95, 96, 97, 98, or 99 percent sequence identity when compared to a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 150, 200, 300, 400, 500 or more contiguous nucleotides in the target RNA (or family members of a given target gene).

As used herein, a non-coding RNA molecules, or their processed small RNA forms, are said to exhibit “complete complementarity” when every nucleotide of one of the sequences read 5′ to 3′ is complementary to every nucleotide of the other sequence when read 3′ to 5′. A nucleotide sequence that is completely complementary to a reference nucleotide sequence will exhibit a sequence identical to the reverse complement sequence of the reference nucleotide sequence.

Methods for determining sequence complementarity are well known in the art and include, but not limited to, bioinformatics tools which are well known in the art (e.g. BLAST, multiple sequence alignment).

According to one embodiment, if the non-coding RNA molecule is or processed into a siRNA, the complementarity is in the range of 90-100% (e.g. 100%) to its target sequence.

According to one embodiment, if the non-coding RNA molecule is or processed into a miRNA or piRNA the complementarity is in the range of 33-100% to its target sequence.

According to one embodiment, if the non-coding RNA molecule is a miRNA, the seed sequence complementarity (i.e. nucleotides 2-8 from the 5′) is in the range of 85-100% (e.g. 100%) to its target sequence.

According to one embodiment, the complementarity to the target sequence is at least about 33% of the processed small RNA form (e.g. 33% of the 21-24 nt). Thus, for example, if the non-coding RNA molecule is a miRNA, 33% of the mature miRNA sequence (e.g. of the 21 nt) comprises seed complementation (e.g. 7 nt out of the 21 nt).

According to one embodiment, the complementarity to the target sequence is at least about 45% of the processed small RNA form (e.g. 45% of the 21-28 nt). Thus, for example, if the non-coding RNA molecule is a miRNA, 45% of the mature miRNA sequence (e.g. 21 nt) comprises seed complementation (e.g. 9-10 nt out of the 21 nt).

According to one embodiment, the non-coding RNA (i.e. prior to modification) is typically selected as one having about 10%, 20%, 30%, 33%, 40%, 50%, 60%, 70%, 80%, 85%, 90%, 95%, 96%, 97%, 98% or up to 99% complementarity towards the sequence of the second target RNA or target RNA of interest.

According to a specific embodiment, the non-coding RNA molecule (i.e. prior to modification) is typically selected as one having no more than 99% complementarity towards the sequence of the second target RNA or target RNA of interest.

According to a specific embodiment, the non-coding RNA molecule (i.e. prior to modification) is typically selected as one having no more than 98% complementarity towards the sequence of the second target RNA or target RNA of interest.

According to a specific embodiment, the non-coding RNA molecule (i.e. prior to modification) is typically selected as one having no more than 97% complementarity towards the sequence of the second target RNA or target RNA of interest.

According to a specific embodiment, the non-coding RNA molecule (i.e. prior to modification) is typically selected as one having no more than 96% complementarity towards the sequence of the second target RNA or target RNA of interest.

According to a specific embodiment, the non-coding RNA molecule (i.e. prior to modification) is typically selected as one having no more than 95% complementarity towards the sequence of the second target RNA or target RNA of interest.

According to a specific embodiment, the non-coding RNA molecule (i.e. prior to modification) is typically selected as one having no more than 90% complementarity towards the sequence of the second target RNA or target RNA of interest.

According to a specific embodiment, the non-coding RNA molecule (i.e. prior to modification) is typically selected as one having no more than 85% complementarity towards the sequence of the second target RNA or target RNA of interest.

According to a specific embodiment, the non-coding RNA molecule (i.e. prior to modification) is typically selected as one having no more than 50% complementarity towards the sequence of the second target RNA or target RNA of interest.

According to a specific embodiment, the non-coding RNA molecule (i.e. prior to modification) is typically selected as one having no more than 33% complementarity towards the sequence of the second target RNA or target RNA of interest.

According to one embodiment, the non-coding RNA molecule (e.g. RNA silencing molecule) is designed so as to comprise at least about 33%, 40%, 45%, 50%, 60%, 70%, 80%, 85%, 90%, 91%, 92%, 93%, 94%, 95%, 96%, 97%, 98%, 99% or even 100% complementarity towards the sequence of the second target RNA or target RNA of interest.

According to a specific embodiment, the non-coding RNA molecule (e.g. RNA silencing molecule) is designed so as to comprise a minimum of 33% complementarity towards the second target RNA or target RNA of interest (e.g. 85-100% seed match).

According to a specific embodiment, the non-coding RNA molecule (e.g. RNA silencing molecule) is designed so as to comprise a minimum of 40% complementarity towards the second target RNA or target RNA of interest.

According to a specific embodiment, the non-coding RNA molecule (e.g. RNA silencing molecule) is designed so as to comprise a minimum of 45% complementarity towards the second target RNA or target RNA of interest.

According to a specific embodiment, the non-coding RNA molecule (e.g. RNA silencing molecule) is designed so as to comprise a minimum of 50% complementarity towards the second target RNA or target RNA of interest.

According to a specific embodiment, the non-coding RNA molecule (e.g. RNA silencing molecule) is designed so as to comprise a minimum of 60% complementarity towards the second target RNA or target RNA of interest.

According to a specific embodiment, the non-coding RNA molecule (e.g. RNA silencing molecule) is designed so as to comprise a minimum of 70% complementarity towards the second target RNA or target RNA of interest.

According to a specific embodiment, the non-coding RNA molecule (e.g. RNA silencing molecule) is designed so as to comprise a minimum of 80% complementarity towards the second target RNA or target RNA of interest.

According to a specific embodiment, the non-coding RNA molecule (e.g. RNA silencing molecule) is designed so as to comprise a minimum of 85% complementarity towards the second target RNA or target RNA of interest.

According to a specific embodiment, the non-coding RNA molecule (e.g. RNA silencing molecule) is designed so as to comprise a minimum of 90% complementarity towards the second target RNA or target RNA of interest.

According to a specific embodiment, the non-coding RNA molecule (e.g. RNA silencing molecule) is designed so as to comprise a minimum of 95% complementarity towards the second target RNA or target RNA of interest.

According to a specific embodiment, the non-coding RNA molecule (e.g. RNA silencing molecule) is designed so as to comprise a minimum of 96% complementarity towards the second target RNA or target RNA of interest.

According to a specific embodiment, the non-coding RNA molecule (e.g. RNA silencing molecule) is designed so as to comprise a minimum of 97% complementarity towards the second target RNA or target RNA of interest.

According to a specific embodiment, the non-coding RNA molecule (e.g. RNA silencing molecule) is designed so as to comprise a minimum of 98% complementarity towards the second target RNA or target RNA of interest.

According to a specific embodiment, the non-coding RNA molecule (e.g. RNA silencing molecule) is designed so as to comprise a minimum of 99% complementarity towards the second target RNA or target RNA of interest.

According to a specific embodiment, the non-coding RNA molecule (e.g. RNA silencing molecule) is designed so as to comprise 100% complementarity towards the second target RNA or target RNA of interest.

According to one embodiment, the non-coding RNA molecule (e.g. RNA silencing molecule) is modified in the guide strand (silencing strand) as to comprise about 50-100% complementarity towards the second target RNA or target RNA of interest.

According to one embodiment, the non-coding RNA molecule (e.g. RNA silencing molecule) is modified in the passenger strand (the complementary strand) as to comprise about 50-100% complementarity towards the second target RNA or target RNA of interest.

In order to generate silencing activity and/or specificity of a non-coding RNA molecule or redirect a silencing activity and/or specificity of a non-coding RNA molecule (e.g. RNA silencing molecule) towards a second target RNA or target RNA of interest, the gene encoding a non-coding RNA molecule (e.g. RNA silencing molecule) is modified using a DNA or RNA editing agent.

Following is a description of various non-limiting examples of methods, DNA editing agents and RNA editing agents used to introduce nucleic acid alterations to a gene encoding a non-coding RNA molecule (e.g. RNA silencing molecule), or to a transcript thereof, and agents for implementing same that can be used according to specific embodiments of the present disclosure.

Genome Editing using engineered endonucleases—this approach refers to a reverse genetics method using artificially engineered nucleases to typically cut and create specific double-stranded breaks (DSBs) at a desired location(s) in the genome, which are then repaired by cellular endogenous processes such as, homologous recombination (HR) or non-homologous end-joining (NHEJ). NHEJ directly joins the DNA ends in a double-stranded break (DSB) with or without minimal ends trimming, while HR utilizes a homologous donor sequence as a template (i.e. the sister chromatid formed during S-phase) for regenerating/copying the missing DNA sequence at the break site. In order to introduce specific nucleotide modifications to the genomic DNA, a donor DNA repair template containing the desired sequence must be present during HR (exogenously provided single stranded or double stranded DNA).

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks (DSBs), several distinct classes of nucleases have been discovered and bioengineered to date. These include the meganucleases, Zinc finger nucleases (ZFNs), transcription-activator like effector nucleases (TALENs) and CRISPR/Cas9 system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks (DSBs) in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. No. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (DSBs) (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break (DSB).

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break (DSB). Repair of these double-stranded breaks (DSBs) through the non-homologous end-joining (NHEJ) pathway often results in small deletions or small sequence insertions (Indels). Since each repair made by NHEJ is unique, the use of a single nuclease pair can produce an allelic series with a range of different insertions or deletions at the target site.

In general NHEJ is relatively accurate (about 75-85% of DSBs in human cells are repaired by NHEJ within about 30 min from detection) in gene editing erroneous NHEJ is relied upon as when the repair is accurate the nuclease will keep cutting until the repair product is mutagenic and the recognition/cut site/PAM motif is gone/mutated or that the transiently introduced nuclease is no longer present.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break (DSB) can be repaired via homologous recombination (HR) (e.g. in the presence of a donor template) to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system and all its variants (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form a RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821).

It was further demonstrated that a synthetic chimeric guide RNA (sgRNA) composed of a fusion between crRNA and tracrRNA could direct Cas9 to cleave DNA targets that are complementary to the crRNA in vitro. It was also demonstrated that transient expression of Cas9 in conjunction with synthetic sgRNAs can be used to produce targeted double-stranded breaks (DSBs) in a variety of different species (Cho et al., 2013; Cong et al., 2013; DiCarlo et al., 2013; Hwang et al., 2013a,b; Jinek et al., 2013; Mali et al., 2013).

The CRISPR/Cas system for genome editing contains two distinct components: a sgRNA and an endonuclease e.g. Cas9.

The sgRNA (also referred to herein as short guide RNA (sgRNA)) is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the sgRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks (DSBs) in the genomic DNA.

A significant advantage of CRISPR/Cas is that the high efficiency of this system is coupled with the ability to easily create synthetic sgRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the sgRNA sequence and the genomic DNA target sequence allows imperfect matches to the target sequence to be cut by Cas9.

Modified versions of the Cas9 enzyme containing a single inactive catalytic domain, either RuvC− or HNH−, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only, PARP (sensor) and XRCC1/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas9 nickase to create a double-nick by designing two sgRNAs with target sequences in close proximity and on opposite strands of the genomic DNA would decrease off-target effect as either sgRNA alone will result in nicks that are not likely to change the genomic DNA, even though these events are not impossible.

Modified versions of the Cas9 enzyme containing two inactive catalytic domains (dead Cas9, or dCas9) have no nuclease activity while still able to bind to DNA based on sgRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Additional variants of Cas9 which may be used by some embodiments of the invention include, but are not limited to, CasX and Cpf1. CasX enzymes comprise a distinct family of RNA-guided genome editors which are smaller in size compared to Cas9 and are found in bacteria (which is typically not found in humans), hence, are less likely to provoke the immune system/response in a human. Also, CasX utilizes a different PAM motif compared to Cas9 and therefore can be used to target sequences in which Cas9 PAM motifs are not found [see Liu J J et al., Nature. (2019) 566(7743):218-2231. Cpf1, also referred to as Cas12a, is especially advantageous for editing AT rich regions in which Cas9 PAMs (NGG) are much less abundant [see Li T et al., Biotechnol Adv. (2019) 37(1):21-27; Murugan K et al., Mol Cell. (2017) 68(1):15-25].

According to another embodiment, the CRISPR system may be fused with various effector domains, such as DNA cleavage domains. The DNA cleavage domain can be obtained from any endonuclease or exonuclease. Non-limiting examples of endonucleases from which a DNA cleavage domain can be derived include, but are not limited to, restriction endonucleases and homing endonucleases (see, for example, New England Biolabs Catalog or Belfort et al. (1997) Nucleic Acids Res.). In exemplary embodiments, the cleavage domain of the CRISPR system is a Fokl endonuclease domain or a modified Fokl endonuclease domain. In addition, the use of Homing Endonucleases (HE) is another alternative. HEs are small proteins (<300 amino acids) found in bacteria, archaea, and in unicellular eukaryotes. A distinguishing characteristic of HEs is that they recognize relatively long sequences (14-40 bp) compared to other site-specific endonucleases such as restriction enzymes (4-8 bp). HEs have been historically categorized by small conserved amino acid motifs. At least five such families have been identified: LAGLIDADG; GIY-YIG; HNH; His-Cys Box and PD-(D/E)xK, which are related to EDxHD enzymes and are considered by some as a separate family. At a structural level, the HNH and His-Cys Box share a common fold (designated ββα-metal) as do the PD-(D/E)xK and EDxHD enzymes. The catalytic and DNA recognition strategies for each of the families vary and lend themselves to different degrees to engineering for a variety of applications. See e.g. Methods Mol Biol. (2014) 1123:1-26. Exemplary Homing Endonucleases which may be used according to some embodiments of the invention include, without being limited to, I-CreI, I-TevI, I-HmuI, I-PpoI and I-Ssp68031.

Modified versions of CRISPR, e.g. dead CRISPR (dCRISPR-endonuclease), may also be utilized for CRISPR transcription inhibition (CRISPRi) or CRISPR transcription activation (CRISPRa) see e.g. Kampmann M., ACS Chem Biol. (2018) 13(2):406-416; La Russa M F and Qi L S., Mol Cell Biol. (2015) 35(22):3800-9].

Other versions of CRISPR which may be used according to some embodiments of the invention include genome editing using components from CRISPR systems together with other enzymes to directly install point mutations into cellular DNA or RNA.

Thus, according to one embodiment, the editing agent is DNA or RNA editing agent.

According to one embodiment, the DNA or RNA editing agent elicits base editing.

The term “base editing” as used herein refers to installing point mutations into cellular DNA or RNA without making double-stranded DNA breaks.

In base editing, DNA base editors typically comprise fusions between a catalytically impaired Cas nuclease and a base modification enzyme that operates on single-stranded DNA (ssDNA). Upon binding to its target DNA locus, base pairing between the gRNA and the target DNA strand leads to displacement of a small segment of single-stranded DNA in an ‘R loop’. DNA bases within this ssDNA bubble are modified by the deaminase enzyme. To improve efficiency in eukaryotic cells, the catalytically disabled nuclease also generates a nick in the non-edited DNA strand, inducing cells to repair the non-edited strand using the edited strand as a template.

Two classes of DNA base editor have been described: cytosine base editors (CBEs) convert a C-G base pair into a T-A base pair, and adenine base editors (ABEs) convert an A-T base pair into a G-C base pair. Collectively, CBEs and ABEs can mediate all four possible transition mutations (C to T, A to G, T to C and G to A). Similarly in RNA, targeted adenosine conversion to inosine utilizes both antisense and Cas13-guided RNA-targeting methods.

According to one embodiment, the DNA or RNA editing agent comprises a catalytically inactive endonuclease (e.g. CRISPR-dCas).

According to one embodiment, the catalytically inactive endonuclease is an inactive Cas9 (e.g. dCas9).

According to one embodiment, the catalytically inactive endonuclease is an inactive Cas13 (e.g. dCas13).

According to one embodiment, the DNA or RNA editing agent comprises an enzyme which is capable of epigenetic editing (i.e. providing chemical changes to the DNA, the RNA or the histone proteins).

Exemplary enzymes include, but are not limited to, DNA methyltransferases, methylases, acetyltransferases. More specifically, exemplary enzymes include e.g. DNA (cytosine-5)-methyltransferase 3A (DNMT3a), Histone acetyltransferase p300, Ten-eleven translocation methylcytosine dioxygenase 1 (TET1), Lysine (K)-specific demethylase 1A (LSD1) and Calcium and integrin binding protein 1 (CIB1).

In addition to the catalytically disabled nuclease, the DNA or RNA editing agents of the invention may also comprise a nucleobase deaminase enzyme and/or a DNA glycosylase inhibitor.

According to a specific embodiment, the DNA or RNA editing agents comprise BE1 (APOBEC1-XTEN-dCas9), BE2 (APOBEC1-XTEN-dCas9-UGI) or BE3 (APOBEC-XTEN-dCas9(A840H)-UGI), along with sgRNA. APOBEC1 is a deaminase full length or catalytically active fragment, XTEN is a protein linker, UGI is uracil DNA glycosylase inhibitor to prevent the subsequent U:G mismatch from being repaired back to a C:G base pair and dCas9 (A840H) is a nickase in which the dCas9 was reverted to restore the catalytic activity of the HNH domain which nicks only the non-edited strand, simulating newly synthesized DNA and leading to the desired U:A product.

Additional enzymes which can be used for base editing according to some embodiments of the invention are specified in Rees and Liu, Nature Reviews Genetics (2018) 19:770-788, incorporated herein by reference in its entirety.

There are a number of publicly available tools available to help choose and/or design target sequences as well as lists of bioinformatically determined unique sgRNAs for different genes in different species such as, but not limited to, the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both sgRNA and a Cas endonuclease (e.g. Cas9, Cpf1, CasX) should be expressed or present (e.g., as a ribonucleoprotein complex) in a target cell. The insertion vector can contain both cassettes on a single plasmid or the cassettes are expressed from two separate plasmids. CRISPR plasmids are commercially available such as the px330 plasmid from Addgene (75 Sidney St, Suite 550A • Cambridge, Mass. 02139). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety. Cas endonucleases that can be used to effect DNA editing with sgRNA include, but are not limited to, Cas9, Cpf1, CasX (Zetsche et al., 2015, Cell. 163(3):759-71), C2c1, C2c2, and C2c3 (Shmakov et al., Mol Cell. 2015 Nov. 5; 60(3):385-97).

According to a specific embodiment, the CRISPR comprises a short guide RNA (sgRNA) comprising a nucleic acid sequence as set forth in SEQ ID NOs: 5-6 or SEQ ID Nos 165-236.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, introduced into the cells, and positive selection is performed to isolate homologous recombination mediated events. The DNA carrying the homologous sequence can be provided as a plasmid, single or double stranded oligo. These homologous recombinants contain a local duplication that is separated by intervening vector sequence, including the selection cassette. In the second step, targeted clones are subjected to negative selection to identify cells that have lost the selection cassette via intra-chromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After the system components have been introduced to the cell and positive selection applied, HR mediated events could be identified. Next, a second targeting vector that contains a region of homology with the desired mutation is introduced into targeted clones, and negative selection is applied to remove the selection cassette and introduce the mutation. The final allele contains the desired mutation while eliminating unwanted exogenous sequences.

According to a specific embodiment, the DNA editing agent comprises a DNA targeting module (e.g., gRNA).

According to a specific embodiment, the DNA editing agent does not comprise an endonuclease.

According to a specific embodiment, the DNA editing agent comprises an endonuclease.

According to a specific embodiment, the DNA editing agent comprises a catalytically inactive endonuclease.

According to a specific embodiment, the DNA editing agent comprises a nuclease (e.g. an endonuclease) and a DNA targeting module (e.g., sgRNA).

According to a specific embodiment, the DNA editing agent is CRISPR/endonuclease.

According to a specific embodiment, the DNA editing agent is CRISPR/Cas, e.g. sgRNA and Cas9 or a sgRNA and dCas9.

According to a specific embodiment, the DNA or RNA editing agent elicits base editing.

According to a specific embodiment, the DNA or RNA editing agent comprises an enzyme for epigenetic editing.

According to a specific embodiment, the DNA editing agent is TALEN.

According to a specific embodiment, the DNA editing agent is ZFN.

According to a specific embodiment, the DNA editing agent is meganuclease.

According to one embodiment, the DNA or RNA editing agent is linked to a reporter for monitoring expression in a cell (e.g. eukaryotic cell).

According to one embodiment, the reporter is a fluorescent reporter protein.

The term “a fluorescent protein” refers to a polypeptide that emits fluorescence and is typically detectable by flow cytometry, microscopy or any fluorescent imaging system, therefore can be used as a basis for selection of cells expressing such a protein.

Examples of fluorescent proteins that can be used as reporters are, without being limited to, the Green Fluorescent Protein (GFP), the Blue Fluorescent Protein (BFP) and the red fluorescent proteins (e.g. dsRed, mCherry, RFP). A non-limiting list of fluorescent or other reporters includes proteins detectable by luminescence (e.g. luciferase) or colorimetric assay (e.g. GUS). According to a specific embodiment, the fluorescent reporter is a red fluorescent protein (e.g. dsRed, mCherry, RFP) or GFP.

A review of new classes of fluorescent proteins and applications can be found in Trends in Biochemical Sciences [Rodriguez, Erik A.; Campbell, Robert E.; Lin, John Y; Lin, Michael Z.; Miyawaki, Atsushi; Palmer, Amy E.; Shu, Xiaokun; Zhang, Jin; Tsien, Roger Y. “The Growing and Glowing Toolbox of Fluorescent and Photoactive Proteins”. Trends in Biochemical Sciences. doi:10.1016/j.tibs.2016.09.010].

According to another embodiment, the reporter is an endogenous gene of a plant. An exemplary reporter is the phytoene desaturase gene (PDS3) which encodes one of the important enzymes in the carotenoid biosynthesis pathway. Its silencing produces an albino/bleached phenotype. Accordingly, plants with reduced expression of PDS3 exhibit reduced chlorophyll levels, up to complete albino and dwarfism. Additional genes which can be used in accordance with the present teachings include, but are not limited to, genes which take part in crop protection.

According to another embodiment, the reporter is an antibiotic selection marker. Examples of antibiotic selection markers that can be used as reporters are, without being limited to, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes.

It will be appreciated that the enzyme NPTII inactivates by phosphorylation a number of aminoglycoside antibiotics such as kanamycin, neomycin, geneticin (or G418) and paromomycin. Of these, kanamycin, neomycin and paromomycin are used in a diverse range of plant species, and G418 is routinely used for selection of transformed mammalian cells.

According to another embodiment, the reporter is a toxic selection marker. An exemplary toxic selection marker that can be used as a reporter is, without being limited to, allyl alcohol selection using the Alcohol dehydrogenase (ADH1) gene. ADH1, comprising a group of dehydrogenase enzymes which catalyse the interconversion between alcohols and aldehydes or ketones with the concomitant reduction of NAD+ or NADP+, breaks down alcoholic toxic substances within tissues. Plants harbouring reduced ADH1 expression exhibit increase tolerance to allyl alcohol. Accordingly, plants with reduced ADH1 are resistant to the toxic effect of allyl alcohol.

Regardless of the DNA editing agent used, the method of the invention is employed such that the gene encoding the non-coding RNA molecule (e.g. RNA silencing molecule) is modified by at least one of a deletion, an insertion or a point mutation.

According to one embodiment, the modification is in a structured region of the non-coding RNA molecule or the RNA silencing molecule.

According to one embodiment, the modification is in a stem region of the non-coding RNA molecule or the RNA silencing molecule.

According to one embodiment, the modification is in a loop region of the non-coding RNA molecule or the RNA silencing molecule.

According to one embodiment, the modification is in a stem region and a loop region of the non-coding RNA molecule or the RNA silencing molecule.

According to one embodiment, the modification is in a non-structured region of the non-coding RNA molecule or the RNA silencing molecule.

According to one embodiment, the modification is in a stem region and a loop region and in non-structured region of the non-coding RNA molecule or the RNA silencing molecule.

According to a specific embodiment, the modification comprises a modification of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the modification comprises a modification of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the native non-coding RNA molecule, e.g. RNA silencing molecule).

According to one embodiment, the modification can be in a consecutive nucleic acid sequence (e.g. at least 5, 10, 20, 30, 40, 50, 100, 150, 200, 300, 400, 500 bases).

According to one embodiment, the modification can be in a non-consecutive manner, e.g. throughout a 20, 50, 100, 150, 200, 500, 1000, 2000, 5000 nucleic acid sequence.

According to a specific embodiment, the modification comprises a modification of at most 200 nucleotides.

According to a specific embodiment, the modification comprises a modification of at most 150 nucleotides.

According to a specific embodiment, the modification comprises a modification of at most 100 nucleotides.

According to a specific embodiment, the modification comprises a modification of at most 50 nucleotides.

According to a specific embodiment, the modification comprises a modification of at most 25 nucleotides.

According to a specific embodiment, the modification comprises a modification of at most 24 nucleotides.

According to a specific embodiment, the modification comprises a modification of at most 23 nucleotides.

According to a specific embodiment, the modification comprises a modification of at most 22 nucleotides.

According to a specific embodiment, the modification comprises a modification of at most 21 nucleotides.

According to a specific embodiment, the modification comprises a modification of at most 20 nucleotides.

According to a specific embodiment, the modification comprises a modification of at most 15 nucleotides.

According to a specific embodiment, the modification comprises a modification of at most 10 nucleotides.

According to a specific embodiment, the modification comprises a modification of at most 5 nucleotides.

According to one embodiment, the modification is such that the recognition/cut site/PAM motif of the RNA silencing molecule is modified to abolish the original PAM recognition site.

According to a specific embodiment, the modification is in at least 1, 2, 3, 4, 5, 6, 7, 8, 9, 10 or more nucleic acids in a PAM motif.

According to one embodiment, the modification comprises an insertion.

According to a specific embodiment, the insertion comprises an insertion of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the insertion comprises an insertion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400 or at most 500 nucleotides (as compared to the native non-coding RNA molecule, e.g. RNA silencing molecule).

According to a specific embodiment, the insertion comprises an insertion of at most 200 nucleotides.

According to a specific embodiment, the insertion comprises an insertion of at most 150 nucleotides.

According to a specific embodiment, the insertion comprises an insertion of at most 100 nucleotides.

According to a specific embodiment, the insertion comprises an insertion of at most 50 nucleotides.

According to a specific embodiment, the insertion comprises an insertion of at most 25 nucleotides.

According to a specific embodiment, the insertion comprises an insertion of at most 24 nucleotides.

According to a specific embodiment, the insertion comprises an insertion of at most 23 nucleotides.

According to a specific embodiment, the insertion comprises an insertion of at most 22 nucleotides.

According to a specific embodiment, the insertion comprises an insertion of at most 21 nucleotides.

According to a specific embodiment, the insertion comprises an insertion of at most 20 nucleotides.

According to a specific embodiment, the insertion comprises an insertion of at most 15 nucleotides.

According to a specific embodiment, the insertion comprises an insertion of at most 10 nucleotides.

According to a specific embodiment, the insertion comprises an insertion of at most 5 nucleotides.

According to one embodiment, the modification comprises a deletion.

According to a specific embodiment, the deletion comprises a deletion of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the deletion comprises a deletion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the native non-coding RNA molecule, e.g. RNA silencing molecule).

According to a specific embodiment, the deletion comprises a deletion of at most 200 nucleotides.

According to a specific embodiment, the deletion comprises a deletion of at most 150 nucleotides.

According to a specific embodiment, the deletion comprises a deletion of at most 100 nucleotides.

According to a specific embodiment, the deletion comprises a deletion of at most 50 nucleotides.

According to a specific embodiment, the deletion comprises a deletion of at most 25 nucleotides.

According to a specific embodiment, the deletion comprises a deletion of at most 24 nucleotides.

According to a specific embodiment, the deletion comprises a deletion of at most 23 nucleotides.

According to a specific embodiment, the deletion comprises a deletion of at most 22 nucleotides.

According to a specific embodiment, the deletion comprises a deletion of at most 21 nucleotides.

According to a specific embodiment, the deletion comprises a deletion of at most 20 nucleotides.

According to a specific embodiment, the deletion comprises a deletion of at most 15 nucleotides.

According to a specific embodiment, the deletion comprises a deletion of at most 10 nucleotides.

According to a specific embodiment, the deletion comprises a deletion of at most 5 nucleotides.

According to one embodiment, the modification comprises a point mutation.

According to a specific embodiment, the point mutation comprises a point mutation of about 1-500 nucleotides, about 1-250 nucleotides, about 1-150 nucleotides, about 1-100 nucleotides, about 1-50 nucleotides, about 1-25 nucleotides, about 1-10 nucleotides, about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the point mutation comprises a point mutation in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the native non-coding RNA molecule, e.g. RNA silencing molecule).

According to a specific embodiment, the point mutation comprises a point mutation in at most 200 nucleotides.

According to a specific embodiment, the point mutation comprises a point mutation in at most 150 nucleotides.

According to a specific embodiment, the point mutation comprises a point mutation in at most 100 nucleotides.

According to a specific embodiment, the point mutation comprises a point mutation in at most 50 nucleotides.

According to a specific embodiment, the point mutation comprises a point mutation in at most 25 nucleotides.

According to a specific embodiment, the point mutation comprises a point mutation in at most 24 nucleotides.

According to a specific embodiment, the point mutation comprises a point mutation in at most 23 nucleotides.

According to a specific embodiment, the point mutation comprises a point mutation in at most 22 nucleotides.

According to a specific embodiment, the point mutation comprises a point mutation in at most 21 nucleotides.

According to a specific embodiment, the point mutation comprises a point mutation in at most 20 nucleotides.

According to a specific embodiment, the point mutation comprises a point mutation in at most 15 nucleotides.

According to a specific embodiment, the point mutation comprises a point mutation in at most 10 nucleotides.

According to a specific embodiment, the point mutation comprises a point mutation in at most 5 nucleotides.

According to one embodiment, the modification comprises a combination of any of a deletion, an insertion and/or a point mutation.

According to one embodiment, the modification comprises nucleotide replacement (e.g. nucleotide swapping).

According to a specific embodiment, the swapping comprises swapping of about 1-500 nucleotides, 1-450 nucleotides, 1-400 nucleotides, 1-350 nucleotides, 1-300 nucleotides, 1-250 nucleotides, 1-200 nucleotides, 1-150 nucleotides, 1-100 nucleotides, 1-90 nucleotides, 1-80 nucleotides, 1-70 nucleotides, 1-60 nucleotides, 1-50 nucleotides, 1-40 nucleotides, 1-30 nucleotides, 1-20 nucleotides, 1-10 nucleotides, 10-100 nucleotides, 10-90 nucleotides, 10-80 nucleotides, 10-70 nucleotides, 10-60 nucleotides, 10-50 nucleotides, 10-40 nucleotides, 10-30 nucleotides, 10-20 nucleotides, 10-15 nucleotides, 20-30 nucleotides, 20-50 nucleotides, 20-70 nucleotides, 30-40 nucleotides, 30-50 nucleotides, 30-70 nucleotides, 40-50 nucleotides, 40-80 nucleotides, 50-60 nucleotides, 50-70 nucleotides, 50-90 nucleotides, 60-70 nucleotides, 60-80 nucleotides, 70-80 nucleotides, 70-90 nucleotides, 80-90 nucleotides, 90-100 nucleotides, 100-110 nucleotides, 100-120 nucleotides, 100-130 nucleotides, 100-140 nucleotides, 100-150 nucleotides, 100-160 nucleotides, 100-170 nucleotides, 100-180 nucleotides, 100-190 nucleotides, 100-200 nucleotides, 110-120 nucleotides, 120-130 nucleotides, 130-140 nucleotides, 140-150 nucleotides, 160-170 nucleotides, 180-190 nucleotides, 190-200 nucleotides, 200-250 nucleotides, 250-300 nucleotides, 300-350 nucleotides, 350-400 nucleotides, 400-450 nucleotides, or about 450-500 nucleotides (as compared to the native non-coding RNA molecule, e.g. RNA silencing molecule).

According to one embodiment, the nucleotide swap comprises a nucleotide replacement in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 250, 300, 350, 400, 450 or at most 500 nucleotides (as compared to the native non-coding RNA molecule, e.g. RNA silencing molecule).

According to a specific embodiment, the nucleotide swapping comprises a nucleotide replacement in at most 200 nucleotides.

According to a specific embodiment, the nucleotide swapping comprises a nucleotide replacement in at most 150 nucleotides.

According to a specific embodiment, the nucleotide swapping comprises a nucleotide replacement in at most 100 nucleotides.

According to a specific embodiment, the nucleotide swapping comprises a nucleotide replacement in at most 50 nucleotides.

According to a specific embodiment, the nucleotide swapping comprises a nucleotide replacement in at most 25 nucleotides.

According to a specific embodiment, the nucleotide swapping comprises a nucleotide replacement in at most 24 nucleotides.

According to a specific embodiment, the nucleotide swapping comprises a nucleotide replacement in at most 23 nucleotides.

According to a specific embodiment, the nucleotide swapping comprises a nucleotide replacement in at most 22 nucleotides.

According to a specific embodiment, the nucleotide swapping comprises a nucleotide replacement in at most 21 nucleotides.

According to a specific embodiment, the nucleotide swapping comprises a nucleotide replacement in at most 20 nucleotides.

According to a specific embodiment, the nucleotide swapping comprises a nucleotide replacement in at most 15 nucleotides.

According to a specific embodiment, the nucleotide swapping comprises a nucleotide replacement in at most 10 nucleotides.

According to a specific embodiment, the nucleotide swapping comprises a nucleotide replacement in at most 5 nucleotides.

According to one embodiment, the gene encoding the non-coding RNA molecule (e.g. RNA silencing molecule) is modified by swapping a sequence of an endogenous RNA silencing molecule (e.g. miRNA) with a RNA silencing sequence of choice (e.g. siRNA).

According to a specific embodiment, the sequence of a siRNA used for gene swapping of an endogenous RNA silencing molecule (e.g. miRNA) comprising a nucleic acid sequence selected from the group consisting of SEQ ID NOs: 1-4, SEQ ID Nos: 93-164 or SEQ ID Nos 243-252.

According to one embodiment, the guide strand of the non-coding RNA molecule (e.g. RNA silencing molecule) is modified to preserve originality of structure and keep the same base pairing profile.

According to one embodiment, the passenger strand of the non-coding RNA molecule (e.g. RNA silencing molecule) is modified to preserve originality of structure and keep the same base pairing profile.

As used herein, the term “originality of structure” refers to the secondary RNA structure (i.e. base pairing profile). Keeping the originality of structure is important for correct and efficient biogenesis/processing of the non-coding RNA (e.g. RNA silencing molecule such as siRNA or miRNA) that is structure- and not purely sequence-dependent.

According to one embodiment, the non-coding RNA (e.g. RNA silencing molecule) is modified in the guide strand (silencing strand) as to comprise about 50-100% complementarity to the target RNA (as discussed above) while the passenger strand is modified to preserve the original (unmodified) non-coding RNA structure.

According to one embodiment, the non-coding RNA (e.g. RNA silencing molecule) is modified such that the seed sequence (e.g. for miRNA nucleotides 2-8 from the 5′ terminal) is complimentary to the target sequence.

According to a specific embodiment, the RNA silencing molecule (i.e. RNAi molecule) is designed such that a sequence of the RNAi molecule is modified to preserve originality of structure and to be recognized by cellular RNAi processing and executing factors.

According to one embodiment, any one or combination of the above described modifications can be carried out in order to confer a silencing specificity towards a second target RNA or towards a target RNA of interest.

It will be appreciated that additional mutations can be introduced by additional events of editing (i.e., concomitantly or sequentially).

The DNA or RNA editing agent of the invention may be introduced into eukaryotic cells using DNA delivery methods (e.g. by expression vectors) or using DNA-free methods.

According to one embodiment, the sgRNA (or any other DNA recognition module used, dependent on the DNA editing system that is used) can be provided as RNA to the cell.

Thus, it will be appreciated that the present techniques relate to introducing the DNA editing agent using transient DNA or DNA-free methods such as RNA transfection (e.g. mRNA+sgRNA transfection), or Ribonucleoprotein (RNP) transfection (e.g. protein-RNA complex transfection, e.g. Cas9/gRNA ribonucleoprotein (RNP) complex transfection). Similarly, RNA editing agent may be introduced using any method known in the art such as RNA transfection (e.g. mRNA+sgRNA transfection), or Ribonucleoprotein (RNP) transfection (e.g. protein-RNA complex transfection, e.g. Cas9/gRNA ribonucleoprotein (RNP) complex transfection).

For example, Cas9 can be introduced as a DNA expression plasmid, in vitro transcript (i.e. RNA), or as a recombinant protein bound to the RNA portion in a ribonucleoprotein particle (RNP). sgRNA, for example, can be delivered either as a DNA plasmid or as an in vitro transcript (i.e. RNA).

Any method known in the art for RNA or RNP transfection can be used in accordance with the present teachings, such as, but not limited to microinjection [as described by Cho et al., “Heritable gene knockout in Caenorhabditis elegans by direct injection of Cas9-sgRNA ribonucleoproteins,” Genetics (2013) 195:1177-1180, incorporated herein by reference], electroporation [as described by Kim et al., “Highly efficient RNA-guided genome editing in human cells via delivery of purified Cas9 ribonucleoproteins” Genome Res. (2014) 24:1012-1019, incorporated herein by reference], or lipid-mediated transfection e.g. using liposomes [as described by Zuris et al., “Cationic lipid-mediated delivery of proteins enables efficient protein-based genome editing in vitro and in vivo” Nat Biotechnol. (2014) doi: 10.1038/nbt.3081, incorporated herein by reference]. Additional methods of RNA transfection are described in U.S. Patent Application No. 20160289675, incorporated herein by reference in its entirety.

One advantage of RNA transfection methods of the invention is that RNA transfection is essentially transient and vector-free. A RNA transgene can be delivered to a cell and expressed therein, as a minimal expressing cassette without the need for any additional sequences (e.g. viral sequences).

According to one embodiment, for expression of exogenous DNA or RNA editing agents of the invention in cells, a polynucleotide sequence encoding the DNA or RNA editing agent is ligated into a nucleic acid construct suitable for cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in eukaryotes (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

For expression in a plant cell, the plant promoter employed can be a constitutive promoter, a tissue specific promoter, an inducible promoter, a chimeric promoter or a developmentally regulated promoter.

Examples of preferred promoters useful for the methods of some embodiments of the invention (in plant cells) are presented in Table I, II, III and IV.

TABLE I
Exemplary constitutive promoters for use in the performance
of some embodiments of the invention in plant cells

| | Expression | |
|---|---|---|
| Gene Source | Pattern | Reference |
| Actin | constitutive | McElroy et al, Plant Cell, 2: 163-171, |
| | | 1990 |
| CAMV 35S | constitutive | Odell et al, Nature, 313: 810-812, 1985 |
| CaMV 19S | constitutive | Nilsson et al., Physiol. Plant 100: 456- |
| | | 462, 1997 |
| GOS2 | constitutive | de Pater et al, Plant J Nov; 2(6): 837- |
| | | 44, 1992 |
| ubiquitin | constitutive | Christensen et al, Plant Mol. Biol. 18: |
| | | 675-689, 1992 |
| Rice | constitutive | Bucholz et al, Plant Mol Biol. 25(5): |
| cyclophilin | | 837-43, 1994 |
| Maize H3 | constitutive | Lepetit et al, Mol. Gen. Genet. 231: |
| histone | | 276-285, 1992 |
| Actin 2 | constitutive | An et al, Plant J. 10(1); 107121, 1996 |
| CVMV | constitutive | Lawrenson et al, Gen Biol 16: 258, |
| (Cassava | | 2015 |
| Vein Mosaic | | |
| Virus | | |
| U6 (AtU626; | constitutive | Lawrenson et al, Gen Biol 16: 258, |
| TaU6) | | 2015 |

TABLE II
Exemplary seed-preferred promoters for use in the performance
of some embodiments of the invention in plant cells

| | Expression | |
|---|---|---|
| Gene Source | Pattern | Reference |
| Seed specific | seed | Simon, et al., Plant Mol. Biol. 5. 191, |
| genes | | 1985; Scofield, et al., J. Biol. Chem. |
| | | 262: 12202, 1987; Baszczynski, et al., |
| | | Plant Mol. Biol. 14: 633, 1990. |
| Brazil Nut | seed | Pearson' et al., Plant Mol. Biol. 18: |
| albumin | | 235-245, 1992. |
| legumin | seed | Ellis, et al. Plant Mol. Biol. 10: 203- |
| | | 214, 1988 |
| Glutelin (rice) | seed | Takaiwa, et al., Mol. Gen. Genet. 208: |
| | | 15-22, 1986; Takaiwa, et al., FEBS |
| | | Letts. 221: 43-47, 1987 |
| Zein | seed | Matzke et al Plant Mol Biol, 143). |
| | | 323-32 1990 |
| napA | seed | Stalberg, et al, Planta 199: 515-519, |
| | | 1996 |
| wheat LMW and | endosperm | Mol Gen Genet 216: 81-90, 1989; NAR |
| HMW, glutenin-1 | | 17: 461-2, |
| Wheat SPA | seed | Albanietal, Plant Cell, 9: 171-184, |
| | | 1997 |
| wheat a, b and g | endosperm | EMBO3: 1409-15, 1984 |
| gliadins | | |
| Barley ltrl | endosperm | |
| promoter | | |
| barley B1, C, D | endosperm | Theor Appl Gen 98: 1253-62, 1999; |
| hordein | | Plant J 4: 343-55, 1993; Mol Gen |
| | | Genet 250: 750-60, 1996 |
| Barley DOF | endosperm | Mena et al, The Plant Journal, 116(1): |
| | | 53-62, 1998 |
| Biz2 | endosperm | EP99106056.7 |
| Synthetic | endosperm | Vicente-Carbajosa et al., Plant J. 13: |
| promoter | | 629-640, 1998 |
| rice prolamin | endosperm | Wu et al, Plant Cell Physiology 39(8) |
| NRP33 | | 885-889, 1998 |
| rice -globulin | endosperm | Wu et al, Plant Cell Physiology 398) |
| Glb-1 | | 885-889, 1998 |
| rice OSH1 | emryo | Sato et al, Proc. Nati. Acad. Sci. |
| | | USA, 93: 8117-8122 |
| rice alpha- | endosperm | Nakase et al. Plant Mol. Biol. 33: |
| globulin | | 513-S22, 1997 |
| REB/OHP-1 | | |
| rice ADP-glucose | endosperm | Trans Res 6: 157-68, 1997 |
| PP | | |
| maize ESR gene | endosperm | Plant J 12: 235-46, 1997 |
| family | | |
| sorgum gamma- | endosperm | PMB 32: 1029-35, 1996 |
| kafirin | | |
| KNOX | emryo | Postma-Haarsma ef al, Plant Mol. Biol. |
| | | 39: 257-71, 1999 |
| rice oleosin | Embryo and | Wu et at, J. Biochem., 123: 386, 1998 |
| | aleuton | |
| sunflower | Seed | Cummins, et al., Plant Mol. Biol. 19: |
| oleosin | (embryo and | 873-876, 1992 |
| | dry seed) | |

TABLE III
Exemplary flower-specific promoters for use in
the performance of the invention in plant cells

| | Expression | |
|---|---|---|
| Gene Source | Pattern | Reference |
| AtPRP4 | flowers | www(dot)salus(dot) medium(dot)edu/ |
| | | mmg/tierney/html |
| chalene synthase | flowers | Van der Meer, et al., Plant Mol. Biol. |
| (chsA) | | 15, 95-109, 1990. |
| LAT52 | anther | Twell et al Mol. Gen Genet. 217: 240- |
| | | 245 (1989) |
| apetala- 3 | flowers | |

TABLE IV
Alternative rice promoters for use in the
performance of the invention in plant cells

| PRO # | Gene | Expression |
|---|---|---|
| PR00001 | Metallothionein Mte | transfer layer of embryo + |
| | | calli |
| PR00005 | putative beta-amylase | transfer layer of embryo |
| PR00009 | Putative cellulose synthase | Weak in roots |
| PR00012 | lipase (putative) | |
| PR00014 | Transferase (putative) | |
| PR00016 | peptidyl prolyl cis-trans | |
| | isomerase (putative) | |
| PR00019 | unknown | |
| PR00020 | prp protein (putative) | |
| PR00029 | noduline (putative) | |
| PR00058 | Proteinase inhibitor Rgpi9 | seed |
| PR00061 | beta expansine EXPB9 | Weak in young flowers |
| PR00063 | Structural protein | young tissues + calli + |
| | | embryo |
| PR00069 | xylosidase (putative) | |
| PR00075 | Prolamine 10 Kda | strong in endosperm |
| PR00076 | allergen RA2 | strong in endosperm |
| PR00077 | prolamine RP7 | strong in endosperm |
| PR00078 | CBP80 | |
| PR00079 | starch branching enzyme I | |
| PR00080 | Metallothioneine-like ML2 | transfer layer of embryo + |
| | | calli |
| PR00081 | putative caffeoyl- CoA | shoot |
| | 3-0 methyltransferase | |
| PR00087 | prolamine RM9 | strong in endosperm |
| PR00090 | prolamine RP6 | strong in endosperm |
| PR00091 | prolamine RP5 | strong in endosperm |
| PR00092 | allergen RA5 | |
| PR00095 | putative methionine | embryo |
| | aminopeptidase | |
| PR00098 | ras-related GTP binding | |
| | protein | |
| PR00104 | beta expansine EXPB1 | |
| PR00105 | Glycine rich protein | |
| PR00108 | metallothionein like | |
| | protein (putative) | |
| PR00110 | RCc3 strong root | |
| PR00111 | uclacyanin 3-like protein | weak discrimination center/ |
| | | shoot meristem |
| PR00116 | 26S proteasome regulatory | very weak meristem |
| | particle non-ATPase subunit | specific |
| | 11 | |
| PR00117 | putative 40S ribosomal protein | weak in endosperm |
| PR00122 | chlorophyll a/lo-binding | very weak in shoot |
| | protein precursor (Cab27) | |
| PR00123 | putative protochlorophyllide | Strong leaves |
| | reductase | |
| PR00126 | metallothionein RiCMT | strong discrimination |
| | | center shoot meristem |
| PR00129 | GOS2 | Strong constitutive |
| PR00131 | GOS9 | |
| PR00133 | chitinase Cht-3 | very weak meristem |
| | | specific |
| PR00135 | alpha- globulin | Strong in endosperm |
| PR00136 | alanine aminotransferase | Weak in endosperm |
| PR00138 | Cyclin A2 | |
| PR00139 | Cyclin D2 | |
| PR00140 | Cyclin D3 | |
| PR00141 | Cyclophyllin 2 | Shoot and seed |
| PR00146 | sucrose synthase SS1 (barley) | medium constitutive |
| PR00147 | trypsin inhibitor ITR1 (barley) | weak in endosperm |
| PR00149 | ubiquitine 2 with intron | strong constitutive |
| PR00151 | WSI18 | Embryo and stress |
| PR00156 | HVA22 homologue (putative) | |
| PR00157 | EL2 | |
| PR00169 | aquaporine | medium constitutive in |
| | | young plants |
| PR00170 | High mobility group protein | Strong constitutive |
| PR00171 | reversibly glycosylated | weak constitutive |
| | protein RGP1 | |
| PR00173 | cytosolic MDH | shoot |
| PR00175 | RAB21 | Embryo and stress |
| PR00176 | CDPK7 | |
| PR00177 | Cdc2-1 | very weak in meristem |
| PR00197 | sucrose synthase 3 | |
| PRO0198 | OsVP1 | |
| PRO0200 | OSH1 | very weak in young |
| | | plant meristem |
| PRO0208 | putative chlorophyllase | |
| PRO0210 | OsNRT1 | |
| PRO0211 | EXP3 | |
| PRO0216 | phosphate transporter OjPT1 | |
| PRO0218 | oleosin 18 kd | aleurone + embryo |
| PRO0219 | ubiquitine 2 without intron | |
| PRO0220 | RFL | |
| PRO0221 | maize UBI delta intron | not detected |
| PRO0223 | glutelin-1 | |
| PRO0224 | fragment of prolamin | |
| | RP6 promoter | |
| PRO0225 | 4xABRE | |
| PRO0226 | glutelin OSGLUA3 | |
| PRO0227 | BLZ-2_short (barley) | |
| PR00228 | BLZ-2_long (barley) | |

The inducible promoter is a promoter induced in a specific plant tissue, by a developmental stage or by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

According to one embodiment the promoter is a pathogen-inducible promoter. These promoters direct the expression of genes in plants following infection with a pathogen such as bacteria, fungi, viruses, nematodes and insects. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol 89:245-254; Uknes et al. (1992) Plant Cell 4:645-656; and Van Loon (1985) Plant Mol. Virol. 4:111-116.

According to one embodiment, when more than one promoter is used in the expression vector, the promoters are identical (e.g., all identical, at least two identical).

According to one embodiment, when more than one promoter is used in the expression vector, the promoters are different (e.g., at least two are different, all are different).

According to one embodiment, the promoter in the expression vector for expression in a plant cell includes, but is not limited to, CaMV 35S, 2×CaMV 35S, CaMV 19S, ubiquitin, AtU626 or TaU6.

According to a specific embodiment, the promoter in the expression vector for expression in a plant cell comprises a 35S promoter.

According to a specific embodiment, the promoter in the expression vector for expression in a plant cell comprises a U6 promoter.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

According to a specific embodiment, the expression vector for expression in a plant cell comprises a termination sequence, such as but not limited to, a G7 termination sequence, an AtuNos termination sequence or a CaMV-35S terminator sequence.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding a DNA editing agent can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Stratagene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A+, pMT010/A+, pMAMneo-5, baculovirus pDSVE, and any other vector allowing expression of proteins under the direction of the SV-40 early promoter, SV-40 late promoter, metallothionein promoter, murine mammary tumor virus promoter, Rous sarcoma virus promoter, polyhedrin promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of DNA editing agents since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This contrasts with vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

According to one embodiment the nucleic acid construct for expression in a plant cell is a binary vector. Examples for binary vectors are pBIN19, pBI101, pBinAR, pGPTV, pCAMBIA, pBIB-HYG, pBecks, pGreen or pPZP (Hajukiewicz, P. et al., Plant Mol. Biol. 25, 989 (1994), and Hellens et al, Trends in Plant Science 5, 446 (2000)).

Examples of other vectors to be used in other methods of DNA delivery in a plant cell (e.g. transfection, electroporation, bombardment, viral inoculation as discussed below) are: pGE-sgRNA (Zhang et al. Nat. Comms. 2016 7:12697), pJIT163-Ubi-Cas9 (Wang et al. Nat. Biotechnol 2004 32, 947-951), pICH47742::2x35S-5′UTR-hCas9(STOP)-NOST (Belhan et al. Plant Methods 2013 11; 9(1):39), pAHC25 (Christensen, A. H. & P. H. Quail, 1996. Ubiquitin promoter-based vectors for high-level expression of selectable and/or screenable marker genes in monocotyledonous plants. Transgenic Research 5: 213-218), pHBT-sGFP(S65T)-NOS (Sheen et al. Protein phosphatase activity is required for light-inducible gene expression in maize, EMBO J. 12 (9), 3497-3505 (1993).

According to one embodiment, in order to express a functional DNA editing agent, in cases where the cleaving module (nuclease) is not an integral part of the DNA recognition unit, the expression vector may encode the cleaving module as well as the DNA recognition unit (e.g. sgRNA in the case of CRISPR/Cas).

Alternatively, the cleaving module (nuclease) and the DNA recognition unit (e.g. sgRNA) may be cloned into separate expression vectors. In such a case, at least two different expression vectors must be transformed into the same eukaryotic cell.

Alternatively, when a nuclease is not utilized (i.e. not administered from an exogenous source to the cell), the DNA recognition unit (e.g. sgRNA) may be cloned and expressed using a single expression vector.

According to one embodiment, the DNA editing agent comprises a nucleic acid agent encoding at least one DNA recognition unit (e.g. sgRNA) operatively linked to a cis-acting regulatory element active in eukaryotic cells (e.g., promoter).

According to one embodiment, the nuclease (e.g. endonuclease) and the DNA recognition unit (e.g. sgRNA) are encoded from the same expression vector. Such a vector may comprise a single cis-acting regulatory element active in eukaryotic cells (e.g., promoter) for expression of both the nuclease and the DNA recognition unit. Alternatively, the nuclease and the DNA recognition unit may each be operably linked to a cis-acting regulatory element active in eukaryotic cells (e.g., promoter).

According to one embodiment, the nuclease (e.g. endonuclease) and the DNA recognition unit (e.g. sgRNA) are encoded from different expression vectors whereby each is operably linked to a cis-acting regulatory element active in eukaryotic cells (e.g., promoter).

According to one embodiment, the method of some embodiments of the invention does not comprise introducing into the cell donor oligonucleotides.

According to one embodiment, the method of some embodiments of the invention further comprises introducing into the cell donor oligonucleotides.

According to one embodiment, when the modification is an insertion, the method further comprises introducing into the cell donor oligonucleotides.

According to one embodiment, when the modification is a deletion, the method further comprises introducing into the cell donor oligonucleotides.

According to one embodiment, when the modification is a deletion and insertion (e.g. swapping), the method further comprises introducing into the cell donor oligonucleotides.

According to one embodiment, when the modification is a point mutation, the method further comprises introducing into the cell donor oligonucleotides.

As used herein, the term “donor oligonucleotides” or “donor oligos” refers to exogenous nucleotides, i.e. externally introduced into the cell to generate a precise change in the genome. According to one embodiment, the donor oligonucleotides are synthetic.

According to one embodiment, the donor oligos are RNA oligos.

According to one embodiment, the donor oligos are DNA oligos.

According to one embodiment, the donor oligos are synthetic oligos.

According to one embodiment, the donor oligonucleotides comprise single-stranded donor oligonucleotides (ssODN).

According to one embodiment, the donor oligonucleotides comprise double-stranded donor oligonucleotides (dsODN).

According to one embodiment, the donor oligonucleotides comprise double-stranded DNA (dsDNA).

According to one embodiment, the donor oligonucleotides comprise double-stranded DNA-RNA duplex (DNA-RNA duplex).

According to one embodiment, the donor oligonucleotides comprise double-stranded DNA-RNA hybrid

According to one embodiment, the donor oligonucleotides comprise single-stranded DNA-RNA hybrid.

According to one embodiment, the donor oligonucleotides comprise single-stranded DNA (ssDNA).

According to one embodiment, the donor oligonucleotides comprise double-stranded RNA (dsRNA).

According to one embodiment, the donor oligonucleotides comprise single-stranded RNA (ssRNA).

According to one embodiment, the donor oligonucleotides comprise the DNA or RNA sequence for swapping (as discussed above).

According to one embodiment, the donor oligonucleotides are provided in a non-expressed vector format or oligo.

According to one embodiment, the donor oligonucleotides comprise a DNA donor plasmid (e.g. circular or linearized plasmid).

According to one embodiment, the donor oligonucleotides comprise about 50-5000, about 100-5000, about 250-5000, about 500-5000, about 750-5000, about 1000-5000, about 1500-5000, about 2000-5000, about 2500-5000, about 3000-5000, about 4000-5000, about 50-4000, about 100-4000, about 250-4000, about 500-4000, about 750-4000, about 1000-4000, about 1500-4000, about 2000-4000, about 2500-4000, about 3000-4000, about 50-3000, about 100-3000, about 250-3000, about 500-3000, about 750-3000, about 1000-3000, about 1500-3000, about 2000-3000, about 50-2000, about 100-2000, about 250-2000, about 500-2000, about 750-2000, about 1000-2000, about 1500-2000, about 50-1000, about 100-1000, about 250-1000, about 500-1000, about 750-1000, about 50-750, about 150-750, about 250-750, about 500-750, about 50-500, about 150-500, about 200-500, about 250-500, about 350-500, about 50-250, about 150-250, or about 200-250 nucleotides of single- or double-stranded DNA as well as chimeric DNA-RNA hybrid.

According to a specific embodiment, the donor oligonucleotides comprising the ssODN (e.g. ssDNA or ssRNA) comprise about 200-500 nucleotides.

According to a specific embodiment, the donor oligonucleotides comprising the dsODN (e.g. dsDNA or dsRNA) comprise about 250-5000 nucleotides.

According to one embodiment, for gene swapping of an endogenous RNA silencing molecule (e.g. miRNA) with a RNA silencing sequence of choice (e.g. siRNA), the expression vector, ssODN (e.g. ssDNA or ssRNA) or dsODN (e.g. dsDNA or dsRNA) does not have to be expressed in a cell and could serve as a non-expressing template. According to a specific embodiment, in such a case only the DNA editing agent (e.g. Cas9/sgRNA modules) need to be expressed if provided in a DNA form.

According to some embodiments, for gene editing of an endogenous RNA silencing molecule without the use of a nuclease, the DNA editing agent (e.g., gRNA) may be introduced into the eukaryotic cell with or without (e.g. oligonucleotide donor DNA or RNA, as discussed herein).

According to one embodiment, introducing into the cell donor oligonucleotides is effected using any of the methods described above (e.g. using the expression vectors or RNP transfection).

According to one embodiment, the sgRNA and the DNA donor oligonucleotides are co-introduced into the cell (e.g. eukaryotic cell). It will be appreciated that any additional factors (e.g. nuclease) may be co-introduced therewith.

According to one embodiment, the sgRNA and the DNA donor oligonucleotides are co-introduced into the plant cell (e.g. via bombardment). It will be appreciated that any additional factors (e.g. nuclease) may be co-introduced therewith.

According to one embodiment, the sgRNA is introduced into the cell prior to the DNA donor oligonucleotides (e.g. within a few minutes or a few hours). It will be appreciated that any additional factors (e.g. nuclease) may be introduced prior to, concomitantly with, or following the sgRNA or the DNA donor oligonucleotides.

According to one embodiment, the sgRNA is introduced into the cell subsequent to the DNA donor oligonucleotides (e.g. within a few minutes or a few hours). It will be appreciated that any additional factors (e.g. nuclease) may be introduced prior to, concomitantly with, or following the sgRNA or the DNA donor oligonucleotides.

According to one embodiment, there is provided a composition comprising at least one sgRNA and DNA donor oligonucleotides for genome editing.

According to one embodiment, there is provided a composition comprising at least one sgRNA, a nuclease (e.g. endonuclease) and DNA donor oligonucleotides for genome editing.

According to one embodiment, the at least one sgRNA is operatively linked to a plant expressible promoter.

The DNA editing agents and optionally the donor oligos of some embodiments of the invention can be administered to a single cell, to a group of cells (e.g. plant cells, primary cells or cell lines as discussed above) or to an organism (e.g. plant, mammal, bird, fish, and insect, as discussed above).

Various methods can be used to introduce the expression vector or donor oligos of some embodiments of the invention into eukaryotic cells (e.g. stem cells or plant cells). Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, microinjection, microparticle bombardment, infection with recombinant viral vectors. In addition, see U.S. Pat. Nos. 5,464,764 and 5,487,992 for positive-negative selection methods.

Thus, the delivery of nucleic acids may be introduced into a cell in embodiments of the invention by any method known to those of skill in the art, including, for example and without limitation: by transformation of protoplasts (See, e.g., U.S. Pat. No. 5,508,184); by desiccation/inhibition-mediated DNA uptake (See, e.g., Potrykus et al. (1985) Mol. Gen. Genet. 199:183-8); by electroporation (See, e.g., U.S. Pat. No. 5,384,253); by agitation with silicon carbide fibers (See, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (See, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2; WO2009046384A1; WO2008148223A1) in the methods to deliver DNA, RNA, Peptides and/or proteins or combinations of nucleic acids and peptides into cells.

Other methods of transfection include the use of transfection reagents (e.g. Lipofectin, ThermoFisher), dendrimers (Kukowska-Latallo, J. F. et al., 1996, Proc. Natl. Acad. Sci. USA 93, 4897-902), cell penetrating peptides (Mae et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, for introducing DNA into cells (e.g. plant cells e.g. protoplasts) the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373.

Introduction of nucleic acids to cells (e.g. eukaryotic cells) by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. For gene therapy, the preferred constructs are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers. Other than containing the necessary elements for the transcription and translation of the inserted coding sequence, the expression construct of some embodiments of the invention can also include sequences engineered to enhance stability, production, purification, yield or toxicity of the expressed peptide.

According to a specific embodiment, a bombardment method is used to introduce foreign genes into eukaryotic cells (e.g. non-plant cells, e.g. animal cells, e.g. mammalian cells). According to one embodiment, the method is transient. Bombardment of eukaryotic cells (e.g. mammalian cells) is also taught by Uchida M et al., Biochim Biophys Acta. (2009) 1790(8):754-64, incorporated herein by reference.

According to one embodiment, plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to one embodiment, an agrobacterium-free expression method is used to introduce foreign genes into plant cells. According to one embodiment, the agrobacterium-free expression method is transient. According to a specific embodiment, a bombardment method is used to introduce foreign genes into plant cells. According to another specific embodiment, bombardment of a plant root is used to introduce foreign genes into plant cells. An exemplary bombardment method which can be used in accordance with some embodiments of the invention is discussed in the examples section which follows.

Furthermore, various cloning kits or gene synthesis can be used according to the teachings of some embodiments of the invention.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the genetically identical transformed plants.

Micropropagation is a process of growing new generation plants from a single piece of tissue that has been excised from a selected parent plant or cultivar. This process permits the mass reproduction of plants having the desired trait. The new generated plants are genetically identical to, and have all of the characteristics of, the original plant. Micropropagation (or cloning) allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsulate the viral DNA. If the virus is a RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Regardless of the transformation/infection method employed, the present teachings further select transformed cells comprising a genome editing event.

According to a specific embodiment, selection is carried out such that only cells comprising a successful accurate modification (e.g. swapping, insertion, deletion, point mutation) in the specific locus are selected. Accordingly, cells comprising any event that includes a modification (e.g. an insertion, deletion, point mutation) in an unintended locus are not selected.

According to one embodiment, selection of modified cells can be performed at the phenotypic level, by detection of a molecular event, by detection of a fluorescent reporter, or by growth in the presence of selection (e.g., antibiotic or other selection marker such as resistance to a drug i.e. Nutlin3 in the case of TP53 silencing).

According to one embodiment, selection of modified cells is performed by analyzing the biogenesis and occurrence of the newly edited RNA silencing molecule (e.g. the presence of novel edited miRNA, siRNAs, piRNAs, tasiRNAs, etc).

According to one embodiment, selection of modified cells is performed by analyzing the silencing activity and/or specificity of the RNA silencing molecule, or it's processed small RNA forms, towards a target RNA of interest by validating at least one eukaryotic cell or organism phenotype of the organism that encode the target RNA of interest e.g. cell size, growth rate/inhibition, cell shape, cell membrane integrity, tumor size, tumor shape, tumor vascularization, a pigmentation of an organism, a size of an organism, infection parameters in an organism (such as viral load or bacterial load) or inflammation parameters in an organism (such as fever or redness), plant leaf coloring, e.g. partial or complete loss of chlorophyll in leaves and other organs (bleaching), presence/absence of necrotic patterns, flower coloring, fruit traits (such as shelf life, firmness and flavor), growth rate, plant size (e.g. dwarfism), crop yield, biotic stress resistance (e.g. disease resistance, nematode mortality, beetle's egg laying rate, or other resistant phenotypes associated with any of bacteria, viruses, fungi, parasites, insects, weeds, and cultivated or native plants), crop yield, metabolic profile, fruit trait, biotic stress resistance, abiotic stress resistance (e.g. heat/cold resistance, drought resistance, salt resistance, resistance to allyl alcohol, or resistant to lack of nutrients e.g. Phosphorus (P)).

According to one embodiment, the silencing specificity of the non-coding RNA molecule or RNA silencing molecule is determined genotypically, e.g. by expression of a gene or lack of expression.

According to one embodiment, the silencing specificity of the non-coding RNA molecule or RNA silencing molecule is determined phenotypically.

According to one embodiment, a phenotype of the eukaryotic cell or organism is determined prior to a genotype.

According to one embodiment, a genotype of the eukaryotic cell or organism is determined prior to a phenotype.

According to one embodiment, selection of modified cells is performed by analyzing the silencing activity and/or specificity of non-coding RNA molecule or RNA silencing molecule towards a target RNA of interest by measuring a RNA level of the target RNA of interest. This can be effected using any method known in the art, e.g. by Northern blotting, Nuclease Protection Assays, In Situ hybridization, quantitative RT-PCR or immunoblotting.

According to one embodiment, selection of modified cells is performed by analyzing eukaryotic cells or clones comprising the DNA editing event also referred to herein as “mutation” or “edit”, dependent on the type of editing sought e.g., insertion, deletion, insertion-deletion (Indel), inversion, substitution and combinations thereof.

Methods for detecting sequence alteration are well known in the art and include, but not limited to, DNA and RNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Heteroduplex and Sanger sequencing, or PCR followed by restriction digest to detect appearance or disappearance of unique restriction site/s.

Another method of validating the presence of a DNA editing event e.g., Indels comprises a mismatch cleavage assay that makes use of a structure selective enzyme (e.g. endonuclease) that recognizes and cleaves mismatched DNA.

According to one embodiment, selection of transformed cells is effected by flow cytometry (FACS) selecting transformed cells exhibiting fluorescence emitted by the fluorescent reporter. Following FACS sorting, positively selected pools of transformed eukaryotic cells, displaying the fluorescent marker are collected and an aliquot can be used for testing the DNA editing event as discussed above.

In cases where antibiotic selection marker was used, following transformation eukaryotic cell are cultivated in the presence of selection (e.g., antibiotic), e.g. in a cell culture or until the plant cells develop into colonies i.e., clones and micro-calli. A portion of the cells of the cell culture or of the calli are then analyzed (validated) for the DNA editing event, as discussed above.

According to one embodiment of the invention, the method further comprises validating in the transformed cells complementarity of the endogenous non-coding RNA molecule or RNA silencing molecule towards the target RNA of interest.

As mentioned above, following modification of the gene encoding the non-coding RNA molecule or RNA silencing molecule, the non-coding RNA molecule or the RNA silencing molecule comprises at least about 30%, 33%, 40%, 50%, 60%, 70%, 80%, 85%, 90%, 91%, 92%, 93%, 94%, 95%, 96%, 97%, 98%, 99% or even 100% complementarity towards the sequence of the target RNA of interest.

The specific binding of designed RNA silencing molecule, or it's processed small RNA forms, with a target RNA of interest can be determined by any method known in the art, such as by computational algorithms (e.g. BLAST) and verified by methods including e.g. Northern blot, In Situ hybridization, QuantiGene Plex Assay etc.

It will be appreciated that positive eukaryotic cells or clones (e.g. plant cell clones) can be homozygous or heterozygous for the DNA editing event. In case of a heterozygous cell, the cell (e.g., when diploid plant cell) may comprise a copy of a modified gene and a copy of a non-modified gene of the RNA silencing molecule. The skilled artisan will select the cells for further culturing/regeneration according to the intended use.

According to one embodiment, when a transient method is desired, eukaryotic cells or clones (e.g. plant cell clones) exhibiting the presence of a DNA editing event as desired are further analyzed and selected for the presence of the DNA editing agent, namely, loss of DNA sequences encoding for the DNA editing agent. This can be done, for example, by analyzing the loss of expression of the DNA editing agent (e.g., at the mRNA, protein) e.g., by fluorescent detection of GFP or q-PCR, HPLC.

According to one embodiment, when a transient method is desired, the eukaryotic cells or clones (e.g. plant cell clones) may be analyzed for the presence of the nucleic acid construct as described herein or portions thereof e.g., nucleic acid sequence encoding the DNA editing agent. This can be affirmed by fluorescent microscopy, q-PCR, FACS, and or any other method such as Southern blot, PCR, sequencing, HPLC).

Positive eukaryotic cell clones may be stored (e.g., cryopreserved).

Alternatively, eukaryotic cells may be further cultured and maintained, for example, in an undifferentiated state for extended periods of time or may be induced to differentiate into other cell types, tissues, organs or organisms as required.

According to one embodiment, when the eukaryotic organism is a plant, the plant is crossed in order to obtain a plant devoid of the DNA editing agent (e.g. of the endonuclease), as discussed below.

Alternatively, plant cells (e.g., protoplasts) may be regenerated into whole plants first by growing into a group of plant cells that develops into a callus and then by regeneration of shoots (callogenesis) from the callus using plant tissue culture methods. Growth of protoplasts into callus and regeneration of shoots requires the proper balance of plant growth regulators in the tissue culture medium that must be customized for each species of plant.

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin.

The regenerated plants can be subjected to further breeding and selection as the skilled artisan sees fit.

Thus, embodiments of the invention further relate to plants, plant cells and processed product of plants comprising the non-coding RNA molecule or RNA silencing molecule capable of silencing a target RNA of interest generated according to the present teachings.

According to one aspect of the invention, there is provided a method of producing a plant comprising a reduced expression of a housekeeping gene, a dominant gene, a gene comprising a high copy number and/or and a gene associated with cell apoptosis, the method comprising:

(a) breeding the plant of some embodiments of the invention; and

(b) selecting for progeny plants that have reduced expression of the housekeeping gene, the dominant gene, the gene comprising a high copy number, and/or the gene associated with cell apoptosis, and which do not comprise the DNA editing agent,

thereby producing the plant with reduced expression of the housekeeping gene, the dominant gene, and/or the gene comprising a high copy number.

According to one aspect of the invention, there is provided a method producing a plant or plant cell of some embodiments of the invention, comprising growing the plant or plant cell under conditions which allow propagation.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that may be useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Cannabaceae, Cannabis indica, Cannabis, Cannabis sativa, Hemp, industrial Hemp, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medic ago saliva, Metasequoia glyptostroboides, Musa sapientum, banana, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to a specific embodiment, the plant is a crop, a flower or a tree.

According to a specific embodiment, the plant is a woody plant species e.g., Actinidia chinensis (Actinidiaceae), Manihotesculenta (Euphorbiaceae), Firiodendron tulipifera (Magnoliaceae), Populus (Salicaceae), Santalum album (Santalaceae), Ulmus (Ulmaceae) and different species of the Rosaceae (Malus, Prunus, Pyrus) and the Rutaceae (Citrus, Microcitrus), Gymnospermae e.g., Picea glauca and Pinus taeda, forest trees (e.g., Betulaceae, Fagaceae, Gymnospermae and tropical tree species), fruit trees, shrubs or herbs, e.g., (banana, cocoa, coconut, coffee, date, grape and tea) and oil palm.

According to a specific embodiment, the plant is of a tropical crop e.g., coffee, macadamia, banana, pineapple, taro, papaya, mango, barley, beans, cassava, chickpea, cocoa (chocolate), cowpea, maize (corn), millet, rice, sorghum, sugarcane, sweet potato, tobacco, taro, tea, yam.

“Grain,” “seed,” or “bean,” refers to a flowering plant's unit of reproduction, capable of developing into another such plant. As used herein, the terms are used synonymously and interchangeably.

According to a specific embodiment, the plant is a plant cell e.g., plant cell in an embryonic cell suspension.

According to a specific embodiment, the plant comprises a plant cell generated by the method of some embodiments of the invention.

According to one embodiment, breeding comprises crossing or selfing.

The term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “crossing” therefore generally refers to the fertilization of ovules of one individual with pollen from another individual, whereas “selfing” refers to the fertilization of ovules of an individual with pollen from the same individual. Crossing is widely used in plant breeding and results in a mix of genomic information between the two plants crossed one chromosome from the mother and one chromosome from the father. This will result in a new combination of genetically inherited traits.

As mentioned above, the plant may be crossed in order to obtain a plant devoid of undesired factors e.g. DNA editing agent (e.g. endonuclease).

According to one embodiment, the plant is non-genetically modified (non-GMO) plant.

According to one embodiment, the plant is a genetically modified (GMO) plant.

According to one embodiment, there is provided a seed of the plant generated according to the method of some embodiments of the invention.

According to one embodiment, there is provided a method of generating a plant with increased stress tolerance, increased yield, increased growth rate or increased yield quality, the method comprising: (a) breeding the plant of some embodiments of the invention, and (b) selecting for progeny plants that have increased stress tolerance, increased yield, increased growth rate or increased yield quality.

The phrase “stress tolerance” as used herein refers to the ability of a plant to endure a biotic or abiotic stress without suffering a substantial alteration in metabolism, growth, productivity and/or viability.

The phrase “abiotic stress” as used herein refers to the exposure of a plant, plant cell, or the like, to a non-living (“abiotic”) physical or chemical agent that has an adverse effect on metabolism, growth, development, propagation, or survival of the plant (collectively, “growth”). An abiotic stress can be imposed on a plant due, for example, to an environmental factor such as water (e.g., flooding, drought, or dehydration), anaerobic conditions (e.g., a lower level of oxygen or high level of CO₂), abnormal osmotic conditions (e.g. osmotic stress), salinity, or temperature (e.g., hot/heat, cold, freezing, or frost), an exposure to pollutants (e.g. heavy metal toxicity), anaerobiosis, nutrient deficiency (e.g., nitrogen deficiency or limited nitrogen), atmospheric pollution or UV irradiation.

The phrase “biotic stress” as used herein refers to the exposure of a plant, plant cell, or the like, to a living (“biotic”) organism that has an adverse effect on metabolism, growth, development, propagation, or survival of the plant (collectively, “growth”). Biotic stress can be caused by, for example, bacteria, viruses, fungi, parasites, beneficial and harmful insects, weeds, and cultivated or native plants.

The phrase “yield” or “plant yield” as used herein refers to increased plant growth (growth rate), increased crop growth, increased biomass, and/or increased plant product production (including grain, fruit, seeds, etc.).

According to one embodiment, in order to generate a plant with increased stress tolerance, increased yield, increased growth rate or increased yield quality, the non-coding RNA molecule or RNA silencing molecule is designed to target a RNA of interest or second target RNA being of a gene of the plant conferring sensitivity to stress, decreased yield, decreased growth rate or decreased yield quality.

According to one embodiment, exemplary susceptibility plant genes to be targeted (e.g. knocked out) include, but are not limited to, the susceptibility S-genes, such as those residing at genetic loci known as MLO (Mildew Locus O).

According to one embodiment, the plants generated by the present method comprise increased stress tolerance, increased yield, increased yield quality, increased growth rate, by at least about 10%, 20%, 30%, 40%, 50%, 60%, 70%, 80%, 90%, 95% or 100% as compared to plants not generated by the present methods.

Any method known in the art for assessing increased stress tolerance may be used in accordance with the present invention. Exemplary methods of assessing increased stress tolerance include, but are not limited to, downregulation of PagSAP1 in poplar for increased salt stress tolerance as described in Yoon, S K., Bae, E K., Lee, H. et al. Trees (2018) 32: 823. www(dot)doi(dot)org/10.1007/s00468-018-1675-2), and increased drought tolerance in tomato by downregulation of SlbZIP38 (Pan Y et al. Genes 2017, 8, 402; doi:10.3390/genes8120402, incorporated herein by reference.

Any method known in the art for assessing increased yield may be used in accordance with the present invention. Exemplary methods of assessing increased yield include, but are not limited to, reduced DST expression in rice as described in Ar-Rafi Md. Faisal, et al, AJPS> Vol. 8 No. 9, August 2017 DOI: 10.4236/ajps.2017.89149; and downregulation of BnFTA in canola resulted in increased yield as described in Wang Y et al., Mol Plant. 2009 January; 2(1): 191-200.doi: 10.1093/mp/ssn088), both incorporated herein by reference.

Any method known in the art for assessing increased growth rate may be used in accordance with the present invention. Exemplary methods of assessing increased growth rate include, but are not limited to, reduced expression of BIG BROTHER in Arabidopsis or GA2-OXIDASE results in enhance growth and biomass as described in Marcelo de Freitas Lima et al. Biotechnology Research and Innovation (2017) 1,14-25, incorporated herein by reference.

Any method known in the art for assessing increased yield quality may be used in accordance with the present invention. Exemplary methods of assessing increased yield quality include, but are not limited to, down regulation of OsCKX2 in rice results in production of more tillers, more grains, and the grains were heavier as described in Yeh S_Y et al. Rice (N Y). 2015; 8: 36; and reduce OMT levels in many plants, which result in altered lignin accumulation, increase the digestibility of the material for industry purposes as described in Verma S R and Dwivedi U N, South African Journal of Botany Volume 91, March 2014, Pages 107-125, both incorporated herein by reference.

According to one embodiment, the method further enables generation of a plant comprising increased sweetness, increased sugar content, increased flavor, improved ripening control, increased water stress tolerance, increased heat stress tolerance, and increased salt tolerance. One of skill in the art will know how to utilize the methods described herein to choose target RNA sequences for modification.

According to one embodiment, there is provided a method of generating a pathogen or pest tolerant or resistant plant, the method comprising: (a) breeding the plant of some embodiments of the invention, and (b) selecting for progeny plants that are pathogen or pest tolerant or resistant.

According to one embodiment, the target RNA of interest or the second target RNA is of a gene of the plant conferring sensitivity to a pathogen or a pest.

According to one embodiment, the target RNA of interest or the second target RNA is of a gene of a pathogen.

According to one embodiment, the target RNA of interest or the second target RNA is of a gene of a pest.

As used herein the term “pathogen” refers to an organism that negatively affect plants by colonizing, damaging, attacking, or infecting them. Thus, pathogen may affect the growth, development, reproduction, harvest or yield of a plant. This includes organisms that spread disease and/or damage the host and/or compete for host nutrients. Plant pathogens include, but are not limited to, fungi, oomycetes, bacteria, viruses, viroids, virus-like organisms, phytoplasmas, protozoa, nematodes, insects and parasitic plants.

Non-limiting examples of pathogens include, but are not limited to, Roundheaded Borer such as long horned borers; psyllids such as red gum lerp psyllids (Glycaspis brimblecombei), blue gum psyllid, spotted gum lerp psyllids, lemon gum lep psyllids; tortoise beetles; snout beetles; leaf beetles; honey fungus; Thaumastocoris peregrinus; sessile gall wasps (Cynipidae) such as Leptocybe invasa, Ophelimus maskelli and Selitrichodes globules; Foliage-feeding caterpillars such as Omnivorous looper and Orange tortrix; Glassy-winged sharpshooter; and Whiteflies such as Giant whitefly. Other non-limiting examples of pathogens include Aphids such as Chaitophorus spp., Cloudywinged cottonwood and Periphyllus spp.; Armored scales such as Oystershell scale and San Jose scale; Carpenterworm; Clearwing moth borers such as American hornet moth and Western poplar clearwing; Flatheaded borers such as Bronze birch borer and Bronze poplar borer; Foliage-feeding caterpillars such as Fall webworm, Fruit-tree leafroller, Redhumped caterpillar, Satin moth caterpillar, Spiny elm caterpillar, Tent caterpillar, Tussock moths and Western tiger swallowtail; Foliage miners such as Poplar shield bearer; Gall and blister mites such as Cottonwood gall mite; Gall aphids such as Poplar petiolegall aphid; Glassy-winged sharpshooter; Leaf beetles and flea beetles; Mealybugs; Poplar and willow borer; Roundheaded borers; Sawflies; Soft scales such as Black scale, Brown soft scale, Cottony maple scale and European fruit lecanium; Treehoppers such as Buffalo treehopper; and True bugs such as Lace bugs and Lygus bugs.

Other non-limiting examples of viral plant pathogens include, but are not limited to Species: Pea early-browning virus (PEBV), Genus: Tobravirus. Species: Pepper ringspot virus (PepRSV), Genus: Tobravirus. Species: Watermelon mosaic virus (WMV), Genus: Potyvirus and other viruses from the Potyvirus Genus. Species: Tobacco mosaic virus Genus (TMV), Tobamovirus and other viruses from the Tobamovirus Genus. Species: Potato virus X Genus (PVX), Potexvirus and other viruses from the Potexvirus Genus. Thus the present teachings envisage targeting of RNA as well as DNA viruses (e.g. Gemini virus or Bigeminivirus). Geminiviridae viruses which may be targeted include, but are not limited to, Abutilon mosaic bigeminivirus, Ageratum yellow vein bigeminivirus, Bean calico mosaic bigeminivirus, Bean golden mosaic bigeminivirus, Bhendi yellow vein mosaic bigeminivirus, Cassava African mosaic bigeminivirus, Cassava Indian mosaic bigeminivirus, Chino del tomaté bigeminivirus, Cotton leaf crumple bigeminivirus, Cotton leaf curl bigeminivirus, Croton yellow vein mosaic bigeminivirus, Dolichos yellow mosaic bigeminivirus, Euphorbia mosaic bigeminivirus, Horsegram yellow mosaic bigeminivirus, Jatropha mosaic bigeminivirus, Lima bean golden mosaic bigeminivirus, Melon leaf curl bigeminivirus, Mung bean yellow mosaic bigeminivirus, Okra leaf-curl bigeminivirus, Pepper hausteco bigeminivirus, Pepper Texas bigeminivirus, Potato yellow mosaic bigeminivirus, Rhynchosia mosaic bigeminivirus, Serrano golden mosaic bigeminivirus, Squash leaf curl bigeminivirus, Tobacco leaf curl bigeminivirus, Tomato Australian leafcurl bigeminivirus, Tomato golden mosaic bigeminivirus, Tomato Indian leafcurl bigeminivirus, Tomato leaf crumple bigeminivirus, Tomato mottle bigeminivirus, Tomato yellow leaf curl bigeminivirus, Tomato yellow mosaic bigeminivirus, Watermelon chlorotic stunt bigeminivirus and Watermelon curly mottle bigeminivirus.

As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission.

According to one embodiment, the pest is an invertebrate organism.

Exemplary pests include, but are not limited to, insects, nematodes, snails, slugs, spiders, caterpillars, scorpions, mites, ticks, fungi, and the like.

Insect pests include, but are not limited to, insects selected from the orders Coleoptera (e.g. beetles), Diptera (e.g. flies, mosquitoes), Hymenoptera (e.g. sawflies, wasps, bees, and ants), Lepidoptera (e.g. butterflies and moths), Mallophaga (e.g. lice, e.g. chewing lice, biting lice and bird lice), Hemiptera (e.g. true bugs), Homoptera including suborders Sternorrhyncha (e.g. aphids, whiteflies, and scale insects), Auchenorrhyncha (e.g. cicadas, leafhoppers, treehoppers, planthoppers, and spittlebugs), and Coleorrhyncha (e.g. moss bugs and beetle bugs), Orthroptera (e.g. grasshoppers, locusts and crickets, including katydids and wetas), Thysanoptera (e.g. Thrips), Dermaptera (e.g. Earwigs), Isoptera (e.g. Termites), Anoplura (e.g. Sucking lice), Siphonaptera (e.g. Flea), Trichoptera (e.g. caddisflies), etc.

Insect pests of the invention include, but are not limited to, Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctate, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabs, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots. According to one embodiment, the pathogen is a nematode. Exemplary nematodes include, but are not limited to, the burrowing nematode (Radopholus similis), Caenorhabditis elegans, Radopholus arabocoffeae, Pratylenchus coffeae, root-knot nematode (Meloidogyne spp.), cyst nematode (Heterodera and Globodera spp.), root lesion nematode (Pratylenchus spp.), the stem nematode (Ditylenchus dipsaci), the pine wilt nematode (Bursaphelenchus xylophilus), the reniform nematode (Rotylenchulus reniformis), Xiphinema index, Nacobbus aberrans and Aphelenchoides besseyi.

According to one embodiment, the pathogen is a fungus. Exemplary fungi include, but are not limited to, Fusarium oxysporum, Leptosphaeria maculans (Phoma lingam), Sclerotinia sclerotiorum, Pyricularia grisea, Gibberella fujikuroi (Fusarium moniliforme), Magnaporthe oryzae, Botrytis cinereal, Puccinia spp., Fusarium graminearum, Blumeria graminis, Mycosphaerella graminicola, Colletotrichum spp., Ustilago maydis, Melampsora lini, Phakopsora pachyrhizi and Rhizoctonia solani.

According to a specific embodiment, the pest is an ant, a bee, a wasp, a caterpillar, a beetle, a snail, a slug, a nematode, a bug, a fly, a whitefly, a mosquito, a grasshopper, an earwig, an aphid, a scale, a thrip, a spider, a mite, a psyllid, and a scorpion.

According to one embodiment, in order to generate a pathogen or pest resistant or tolerant plant, the non-coding RNA molecule or RNA silencing molecule is designed to target a RNA of interest or a second target RNA being of a gene of the plant conferring sensitivity to a pathogen or the pest.

Preferably, silencing of the pathogen or pest gene results in the suppression, control, and/or killing of the pathogen or pest which results in limiting the damage that the pathogen or pest causes to the plant. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant.

According to one embodiment, an exemplary plant gene to be targeted includes, but is not limited to, the gene eIF4E which confers sensitivity to viral infection in cucumber.

According to one embodiment, in order to generate a pathogen resistant or tolerant plant, the non-coding RNA molecule or RNA silencing molecule is designed to target a RNA of interest or second target RNA being of a gene of the pathogen.

Determination of the plant or pathogen target genes may be achieved using any method known in the art such as by routine bioinformatics analysis.

According to one embodiment, the nematode pathogen gene comprises the Radopholus similis genes Calreticulin13 (CRT) or collagen 5 (col-5).

According to one embodiment, the fungi pathogen gene comprises the Fusarium oxysporum genes FOW2, FRP1, and OPR.

According to one embodiment, the pathogen gene includes, for example, vacuolar ATPase (vATPase), dvssj1 and dvssj2, α-tubulin and snf7.

According to a specific embodiment, when the plant is a Brassica napus (rapeseed), the target RNA of interest or the second target RNA includes, but is not limited to, a gene of Leptosphaeria maculans (Phoma lingam) (causing e.g. Phoma stem canker) (e.g. as set forth in GenBank Accession No: AM933613.1); a gene of Flea beetle (Phyllotreta vittula or Chrysomelidae, e.g. as set forth in GenBank Accession No: KT959245.1); or a gene of by Sclerotinia sclerotiorum (causing e.g. Sclerotinia stem rot) (e.g. as set forth in GenBank Accession No: NW_001820833.1).

According to a specific embodiment, when the plant is a Citrus x sinensis (Orange), the target RNA of interest or the second target RNA includes, but is not limited to, a gene of Citrus Canker (CCK) (e.g. as set forth in GenBank Accession No: AE008925); a gene of Candidatus Liberibacter spp. (causing e.g. Citrus greening disease) (e.g. as set forth in GenBank Accession No: CP001677.5); or a gene of Armillaria root rot (e.g. as set forth in GenBank Accession No: KY389267.1).

According to a specific embodiment, when the plant is a Elaeis guineensis (Oil palm), the target RNA of interest or the second target RNA includes, but is not limited to, a gene of Ganoderma spp. (causing e.g. Basal stem rot (BSR) also known as Ganoderma butt rot) (e.g. as set forth in GenBank Accession No: U56128.1), a gene of Nettle Caterpillar or a gene of any one of Fusarium spp., Phytophthora spp., Pythium spp., Rhizoctonia solani (causing e.g. Root rot).

According to a specific embodiment, when the plant is a Fragaria vesca (Wild strawberry), the target RNA of interest or the second target RNA includes, but is not limited to, a gene of Verticillium dahlia (causing e.g. Verticillium Wilt) (e.g. as set forth in GenBank Accession No: DS572713.1); or a gene of Fusarium oxysporum f. sp. fragariae (causing e.g. Fusarium wilt) (e.g. as set forth in GenBank Accession No: KR855868.1);

According to a specific embodiment, when the plant is a Glycine max (Soybean), the target RNA of interest or the second target RNA includes, but is not limited to, a gene of P. pachyrhizi (causing e.g. Soybean rust, also known as Asian rust) (e.g. as set forth in GenBank Accession No: DQ026061.1); a gene of Soybean Aphid (e.g. as set forth in GenBank Accession No: KJ451424.1); a gene of Soybean Dwarf Virus (SbDV) (e.g. as set forth in GenBank Accession No: NC_003056.1); or a gene of Green Stink Bug (Acrosternum hilare) (e.g. as set forth in GenBank Accession No: NW_020110722.1).

According to a specific embodiment, when the plant is a Gossypium raimondii (Cotton), the target RNA of interest or the second target RNA includes, but is not limited to, a gene of Fusarium oxysporum f. sp. vasinfectum (causing e.g. Fusarium wilt) (e.g. as set forth in GenBank Accession No: JN416614.1); a gene of Soybean Aphid (e.g. as set forth in GenBank Accession No: KJ451424.1); or a gene of Pink bollworm (Pectinophora gossypiella) (e.g. as set forth in GenBank Accession No: KU550964.1).

According to a specific embodiment, when the plant is a Oryza sativa (Rice), the target RNA of interest or the second target RNA includes, but is not limited to, a gene of Pyricularia grisea (causing e.g. Rice Blast) (e.g. as set forth in GenBank Accession No: AF027979.1); a gene of Gibberella fujikuroi (Fusarium moniliforme) (causing e.g. Bakanae Disease) (e.g. as set forth in GenBank Accession No: AY862192.1); or a gene of a Stem borer, e.g. Scirpophaga incertulas Walker—Yellow Stem Borer, S. innota Walker—White Stem Borer, Chilo suppressalis Walker—Striped Stem Borer, Sesamia inferens Walker—Pink Stem Borer (e.g. as set forth in GenBank Accession No: KF290773.1).

According to a specific embodiment, when the plant is a Solanum lycopersicum (Tomato), the target RNA of interest or the second target RNA includes, but is not limited to, a gene of Phytophthora infestans (causing e.g. Late blight) (e.g. as set forth in GenBank Accession No: AY855210.1); a gene of a whitefly Bemisia tabaci (e.g. Gennadius, e.g. as set forth in GenBank Accession No: KX390870.1); or a gene of Tomato yellow leaf curl geminivirus (TYLCV) (e.g. as set forth in GenBank Accession No: LN846610.1).

According to a specific embodiment, when the plant is a Solanum tuberosum (Potato), the target RNA of interest or the second target RNA includes, but is not limited to, a gene of Phytophthora infestans (causing e.g. Late Blight) (e.g., as set forth in GenBank Accession No: AY050538.3); a gene of Erwinia spp. (causing e.g. Blackleg and Soft Rot) (e.g. as set forth in GenBank Accession No: CP001654.1); or a gene of Cyst Nematodes (e.g. Globodera pallida and G. rostochiensis) (e.g. as set forth in GenBank Accession No: KF963519.1).

According to a specific embodiment, when the plant is a Theobroma cacao (Cacao), the target RNA of interest or the second target RNA includes, but is not limited to, a gene of a gene of basidiomycete Moniliophthora roreri (causing e.g. Frosty Pod Rot) (e.g. as set forth in GenBank Accession No: LATX01001521.1); a gene of Moniliophthora perniciosa (causing e.g. Witches' Broom disease); or a gene of Mirids e.g. Distantiella theobroma and Sahlbergella singularis, Helopeltis spp, Monalonion specie.

According to a specific embodiment, when the plant is a Vitis vinifera (Grape or Grapevine), the target RNA of interest or the second target RNA includes, but is not limited to, a gene of closterovirus GVA (causing e.g. Rugose wood disease) (e.g. as set forth in GenBank Accession No: AF007415.2); a gene of Grapevine leafroll virus (e.g. as set forth in GenBank Accession No: FJ436234.1); a gene of Grapevine fanleaf degeneration disease virus (GFLV) (e.g. as set forth in GenBank Accession No: NC_003203.1); or a gene of Grapevine fleck disease (GFkV) (e.g. as set forth in GenBank Accession No: NC_003347.1).

According to a specific embodiment, when the plant is a Zea mays (Maize also referred to as corn), the target RNA of interest or the second target RNA includes, but is not limited to, a gene of a Fall Armyworm (e.g. Spodoptera frugiperda) (e.g. as set forth in GenBank Accession No: AJ488181.3); a gene of European corn borer (e.g. as set forth in GenBank Accession No: GU329524.1); or a gene of Northern and western corn rootworms (e.g. as set forth in GenBank Accession No: NM_001039403.1).

According to a specific embodiment, when the plant is a sugarcane, the target RNA of interest or the second target RNA includes, but is not limited to, a gene of a Internode Borer (e.g. Chilo saccharifagus indicus), a gene of a Xanthomonas albileneans (causing e.g. Leaf Scald) or a gene of a Sugarcane Yellow Leaf Virus (SCYLV).

According to a specific embodiment, when the plant is a wheat, the target RNA of interest or the second target RNA includes, but is not limited to, a gene of a Puccinia striiformis (causing e.g. stripe rust) or a gene of an Aphid.

According to a specific embodiment, when the plant is a barley, the target RNA of interest or the second target RNA includes, but is not limited to, a gene of a Puccinia hordei (causing e.g. Leaf rust), a gene of Puccinia striiformis f. sp. hordei (causing e.g. stripe rust), or a gene of an Aphid.

According to a specific embodiment, when the plant is a sunflower, the target RNA of interest or the second target RNA includes, but is not limited to, a gene of a Puccinia helianthi (causing e.g. Rust disease); a gene of Boerema macdonaldii (causing e.g. Phoma black stem); a gene of a Seed weevil (e.g. red and gray), e.g. Smicronyx fulvus (red); Smicronyx sordidus (gray); or a gene of Sclerotinia sclerotiorum (causing e.g. Sclerotinia stalk and head rot disease).

According to a specific embodiment, when the plant is a rubber plant, the target RNA of interest or the second target RNA includes, but is not limited to, a gene of a Microcyclus ulei (causing e.g. South American leaf blight (SALB)); a gene of Rigidoporus microporus (causing e.g. White root disease); a gene of Ganoderma pseudoferreum (causing e.g. Red root disease).

According to a specific embodiment, when the plant is an apple plant, the target RNA of interest or the second target RNA includes, but is not limited to, a gene of Neonectria ditissima (causing e.g. Apple Canker), a gene of Podosphaera leucotricha (causing e.g. Apple Powdery Mildew), or a gene of Venturia inaequalis (causing e.g. Apple Scab).

According to one embodiment, the plants generated by the present method are more resistant or tolerant to pathogens by at least about 10%, 20%, 30%, 40%, 50%, 60%, 70%, 80%, 90%, 95% or 100% as compared to plants not generated by the present methods (i.e. as compared to wild type plants).

Any method known in the art for assessing tolerance or resistance to a pathogen of a plant may be used in accordance with the present invention. Exemplary methods include, but are not limited to, reducing MYB46 expression in Arabidopsis which results in enhanced resistance to Botrytis cinerea as described in Ramirez V1, Garcia-Andrade J, Vera P., Plant Signal Behav. 2011 June; 6(6):911-3. Epub 2011 Jun. 1; or downregulation of HCT in alfalfa promotes activation of defense response in the plant as described in Gallego-Giraldo L. et al. New Phytologist (2011) 190: 627-639 doi: 10.1111/j.1469-8137.2010.03621.x), both incorporated herein by reference.

According to one embodiment, there is provided a method of generating a herbicide resistant plant, the method comprising: (a) breeding the plant of some embodiments of the invention, and (b) selecting for progeny plants that are herbicide resistant.

According to one embodiment, the herbicides target pathways that reside within plastids (e.g. within the chloroplast).

Thus to generate herbicide resistant plants, the non-coding RNA molecule or RNA silencing molecule is designed to target a RNA of interest or second target RNA including, but not limited to, the chloroplast gene psbA (which codes for the photosynthetic quinone-binding membrane protein QB, the target of the herbicide atrazine) and the gene for EPSP synthase (a nuclear protein, however, its overexpression or accumulation in the chloroplast enables plant resistance to the herbicide glyphosate as it increases the rate of transcription of EPSPs as well as by a reduced turnover of the enzyme).

According to one embodiment, the plants generated by the present method are more resistant to herbicides by at least about 10%, 20%, 30%, 40%, 50%, 60%, 70%, 80%, 90%, 95% or 100% as compared to plants not generated by the present methods.

According to one embodiment, there is provided a plant generated according to the method of some embodiments of the invention.

According to one aspect of the invention, there is provided a method of generating a plant, wherein at least some of the cells of the plant comprise a genome comprising a polynucleotide sequence encoding a non-coding RNA molecule or a silencing molecule having a nucleic acid sequence alteration which results in reduced expression of a housekeeping gene, a dominant gene, a gene comprising a high copy number, and/or a gene associated with cell apoptosis.

According to one embodiment, the reduced expression is by about 10-25%, 10-50%, 10-99%, 20-90%, 25-75%, 30-80%, 40-50%, 50-60%, 60-70%, 70-80% or 90-99% as compared to a plant not generated by the methods of some embodiments of the invention (e.g. wild type plant of the same species).

According to one embodiment, the reduced expression is by about 5%, 10%, 20%, 30%, 40%, 50%, 60%, 70%, 80%, 90%, 95% or 99%, as compared to a plant not generated by the methods of some embodiments of the invention (e.g. wild type plant of the same species).

According to one aspect of the invention, there is provided a method of treating a disease in a subject in need thereof, the method comprising modifying a gene encoding or processed into a non-coding RNA molecule or into an RNA silencing molecule according to the method of some embodiments of the invention, wherein the target RNA of interest or the second target RNA is a transcript of a housekeeping gene, a dominant gene, a gene comprising a high copy number, and/or a gene associated with cell apoptosis, associated with an onset or progression of the disease.

According to one embodiment the disease is an infectious disease, a monogenic recessive disorder, an autoimmune disease and a cancerous disease.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” or “subject in need thereof” includes animals, including mammals, preferably human beings, at any age or gender which suffer from the pathology. Preferably, this term encompasses individuals who are at risk to develop the pathology.

The term “infectious diseases” as used herein refers to any of chronic infectious diseases, subacute infectious diseases, acute infectious diseases, viral diseases, bacterial diseases, protozoan diseases, parasitic diseases, fungal diseases, mycoplasma diseases and prion diseases. According to one embodiment, in order to treat an infectious disease in a subject, the a non-coding RNA molecule or RNA silencing molecule is designed to target a RNA of interest or second target RNA associated with onset or progression of the infectious disease.

According to one embodiment, the target RNA of interest or the second target RNA comprises a product of a gene of the eukaryotic cell conferring resistance to the pathogen (e.g. virus, bacteria, fungi, etc.). Exemplary genes include, but are not limited to, CyPA-(Cyclophilins (CyPs)), Cyclophilin A (e.g. for Hepatitis C virus infection), CD81, scavenger receptor class B type I (SR-BI), ubiquitin specific peptidase 18 (USP18), phosphatidylinositol 4-kinase III alpha (PI4K-IIIα) (e.g. for HSV infection) and CCR5− (e.g. for HIV infection). According to one embodiment, the target RNA of interest or the second target RNA comprises a product of a gene of the pathogen.

According to one embodiment, the virus is an arbovirus (e.g. Vesicular stomatitis Indiana virus—VSV). According to one embodiment, the target RNA of interest or the second target RNA comprises a product of a VSV gene, e.g. G protein (G), large protein (L), phosphoprotein, matrix protein (M) or nucleoprotein.

According to one embodiment, the target RNA of interest or the second target RNA includes but is not limited to gag and/or vif genes (i.e. conserved sequences in HIV-1); P protein (i.e. an essential subunit of the viral RNA-dependent RNA polymerase in RSV); P mRNA (i.e. in PIV); core, NS3, NS4B and NS5B (i.e. in HCV); VAMP-associated protein (hVAP-A), La antigen and polypyrimidine tract binding protein (PTB) (i.e. for HCV).

According to a specific embodiment, when the organism is a human, the target RNA of interest or the second target RNA includes, but is not limited to, a gene of a pathogen causing Malaria; a gene of HIV virus (e.g. as set forth in GenBank Accession No: NC_001802.1); a gene of HCV virus (e.g. as set forth in GenBank Accession No: NC_004102.1); and a gene of Parasitic worms (e.g. as set forth in GenBank Accession No: XM_003371604.1).

According to a specific embodiment, when the organism is a human, the target RNA of interest or the second target RNA includes, but is not limited to, a gene related to a cancerous disease (e.g. Homo sapiens mRNA for bcr/abl e8a2 fusion protein, as set forth in GenBank Accession No: AB069693.1) or a gene related to a myelodysplastic syndrome (MDS) and to vascular diseases (e.g. Human heparin-binding vascular endothelial growth factor (VEGF) mRNA, as set forth in GenBank Accession No: M32977.1)

According to a specific embodiment, when the organism is a cattle, the target RNA of interest or the second target RNA includes, but is not limited to, a gene of Infectious bovine rhinotracheitis virus (e.g. as set forth in GenBank Accession No: AJ004801.1), a type 1 bovine herpesvirus (BHV1), causing e.g. BRD (Bovine Respiratory Disease complex); a gene of Bluetongue disease (BTV virus) (e.g. as set forth in GenBank Accession No: KP821170.1); a gene of Bovine Virus Diarrhhoea (BVD) (e.g. as set forth in GenBank Accession No: NC_001461.1); a gene of picornavirus (e.g. as set forth in GenBank Accession No: NC_004004.1), causing e.g. Foot & Mouth disease; a gene of Parainfluenza virus type 3 (PI3) (e.g. as set forth in GenBank Accession No: NC_028362.1), causing e.g. BRD; a gene of Mycobacterium bovis (M. bovis) (e.g. as set forth in GenBank Accession No: NC_037343.1), causing e.g. Bovine Tuberculosis (bTB).

According to a specific embodiment, when the organism is a sheep, the target RNA of interest or the second target RNA includes, but is not limited to, a gene of a pathogen causing Tapeworms disease (E. granulosus life cycle, Echinococcus granulosus, Taenia ovis, Taenia hydatigena, Moniezia species) (e.g. as set forth in GenBank Accession No: AJ012663.1); a gene of a pathogen causing Flatworms disease (Fasciola hepatica, Fasciola gigantica, Fascioloides magna, Dicrocoelium dendriticum, Schistosoma bovis) (e.g. as set forth in GenBank Accession No: AY644459.1); a gene of a pathogen causing Bluetongue disease (BTV virus, e.g. as set forth in GenBank Accession No: KP821170.1); and a gene of a pathogen causing Roundworms disease (Parasitic bronchitis, also known as ““hoose””, Elaeophora schneideri, Haemonchus contortus, Trichostrongylus species, Teladorsagia circumcincta, Cooperia species, Nematodirus species, Dictyocaulus filaria, Protostrongylus refescens, Muellerius capillaris, Oesophagostomum species, Neostrongylus linearis, Chabertia ovina, Trichuris ovis) (e.g. as set forth in GenBank Accession No: NC_003283.11).

According to a specific embodiment, when the organism is a pig, the target RNA of interest or the second target RNA includes, but is not limited to, a gene of African swine fever virus (ASFV) (causing e.g. African Swine Fever) (e.g. as set forth in GenBank Accession No: NC_001659.2); a gene of Classical swine fever virus (causing e.g. Classical Swine Fever) (e.g. as set forth in GenBank Accession No: NC_002657.1); and a gene of a picornavirus (causing e.g. Foot & Mouth disease) (e.g. as set forth in GenBank Accession No: NC_004004.1).

According to a specific embodiment, when the organism is a chicken, the target RNA of interest or the second target RNA includes, but is not limited to, a gene of Bird flu (or Avian influenza), a gene of a variant of avian paramyxovirus 1 (APMV-1) (causing e.g. Newcastle disease), or a gene of a pathogen causing Marek's disease.

According to a specific embodiment, when the organism is a tadpole shrimp, the target RNA of interest or the second target RNA includes, but is not limited to, a gene of White Spot Syndrome Virus (WSSV), a gene of Yellow Head Virus (YHV), or a gene of Taura Syndrome Virus (TSV).

According to a specific embodiment, when the organism is a salmon, the target RNA of interest or the second target RNA includes, but is not limited to, a gene of Infectious Salmon Anaemia (ISA), a gene of Infectious Hematopoietic Necrosis (IHN), a gene of Sea lice (e.g. ectoparasitic copepods of the genera Lepeophtheirus and Caligus).

Assessing the efficacy of treatment may be carried out using any method known in the art, such as by assessing the subject's physical well-being, by blood tests, by assessing viral/bacterial load, etc.

As used herein, the term “monogenic recessive disorder” refers to a disease or condition caused as a result of a single defective gene on the autosomes.

According to one embodiment, the monogenic recessive disorder is a result of a spontaneous or hereditary mutation.

According to one embodiment, the monogenic recessive disorder is autosomal dominant, autosomal recessive or X-linked recessive.

Exemplary monogenic recessive disorders include, but are not limited to, severe combined immunodeficiency (SCID), hemophilia, enzyme deficiencies, Parkinson's Disease, Wiskott-Aldrich syndrome, Cystic Fibrosis, Phenylketonuria, Friedrich's Ataxia, Duchenne Muscular Dystrophy, Hunter disease, Aicardi Syndrome, Klinefelter's Syndrome, Leber's hereditary optic neuropathy (LHON).

According to one embodiment, in order to treat a monogenic recessive disorder in a subject, the non-coding RNA molecule or RNA silencing molecule is designed to target a RNA of interest or second target RNA associated with the monogenic recessive disorder.

According to one embodiment, when the disorder is Parkinson's disease the target RNA of interest or the second target RNA comprises a product of a SNCA (PARK1=4), LRRK2 (PARK8), Parkin (PARK2), PINK1 (PARK6), DJ-1 (PARK7), or ATP13A2 (PARK9) gene.

According to one embodiment, when the disorder is hemophilia or von Willebrand disease the target RNA of interest or the second target RNA comprises, for example, a product of an anti-thrombin gene, of coagulation factor VIII gene or of factor IX gene.

Assessing the efficacy of treatment may be carried out using any method known in the art, such as by assessing the subject's physical well-being, by blood tests, bone marrow aspirate, etc.

Non-limiting examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and Pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to one embodiment, the autoimmune disease comprises systemic lupus erythematosus (SLE).

According to one embodiment, in order to treat an autoimmune disease in a subject, the non-coding RNA molecule or RNA silencing molecule is designed to target a RNA of interest or second target RNA associated with the autoimmune disease.

According to one embodiment, when the disease is lupus, the target RNA of interest or the second target RNA comprises an antinuclear antibody (ANA) such as that pathologically produced by B cells.

Assessing the efficacy of treatment may be carried out using any method known in the art, such as by assessing the subject's physical well-being, by blood tests, bone marrow aspirate, etc.

Non-limiting examples of cancers which can be treated by the method of some embodiments of the invention can be any solid or non-solid cancer and/or cancer metastasis or precancer, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to one embodiment, the cancer which can be treated by the method of some embodiments of the invention comprises a hematologic malignancy. An exemplary hematologic malignancy comprises one which involves malignant fusion of the ABL tyrosine kinase to different other chromosomes generating what is termed BCR-ABL which in turn resulting in malignant fusion protein. Accordingly, targeting the fusion point in the mRNA may silence only the fusion mRNA for down-regulation while the normal proteins, essential for the cell, will be, spared.

According to one embodiment, in order to treat a cancerous disease in a subject, the non-coding RNA molecule or RNA silencing molecule is designed to target a RNA of interest or second target RNA associated with the cancerous disease.

According to one embodiment, the target RNA of interest or the second target RNA comprises a product of an oncogene (e.g. mutated oncogene).

According to one embodiment, the target RNA of interest or the second target RNA restores the function of a tumor suppressor.

According to one embodiment, the target RNA of interest or the second target RNA comprises a product of a RAS, MCL-1 or MYC gene.

According to one embodiment, the target RNA of interest or the second target RNA comprises a product of a BCL-2 family of apoptosis-related genes.

Exemplary target genes include, but are not limited to, mutant dominant negative TP53, Bcl-x, IAPs, Flip, Faim3 and SMS1.

According to one embodiment, when the cancer is melanoma, the target RNA of interest or the second target RNA comprises BRAF. Several forms of BRAF mutations are contemplated herein, including e.g. V600E, V600K, V600D, V600G, and V600R.

According to one embodiment, the method is affected by targeting non-coding RNA molecules or RNA silencing molecules in healthy immune cells, such as white blood cells e.g. T cells, B cells or NK cells (e.g. from a patient or from a cell donor) to a target a RNA of interest or the second target RNA such that the immune cells are capable of killing (directly or indirectly) malignant cells (e.g. cells of a hematological malignancy).

According to one embodiment, the method is affected by targeting non-coding RNA molecules or RNA silencing molecules to silence proteins (i.e. target RNA of interest) that are manipulated by cancer factors (i.e. in order to suppress immune responses from recognizing the malignancy), such that the cancer can be recognized and eradicated by the native immune system.

Assessing the efficacy of treatment may be carried out using any method known in the art, such as by assessing the tumor growth or the number of neoplasms or metastases, e.g. by MRI, CT, PET-CT, by blood tests, ultrasound, x-ray, etc.

According to one aspect of the invention, there is provided a method of enhancing efficacy and/or specificity of a chemotherapeutic agent in a subject in need thereof, the method comprising modifying a gene encoding or processed into a non-coding RNA molecule or into an RNA silencing molecule according to the method of some embodiments of the invention, wherein the non-coding RNA molecule or RNA silencing molecule comprises a silencing activity towards a transcript of a gene associated with an enhancement of efficacy and/or specificity of the chemotherapeutic agent.

According to one aspect of the invention, there is provided a method of enhancing efficacy and/or specificity of a chemotherapeutic agent in a subject in need thereof, the method comprising modifying a gene encoding or processed into a non-coding RNA molecule or into an RNA silencing molecule according to the method of some embodiments of the invention, wherein the target RNA of interest or the second target RNA is a transcript of a housekeeping gene, a dominant gene, a gene comprising a high copy number, and/or a gene associated with cell apoptosis, associated with an onset or progression of the disease.

As used herein, the term “chemotherapeutic agent” refer to an agent that reduces, prevents, mitigates, limits, and/or delays the growth of neoplasms or metastases, or kills neoplastic cells directly by necrosis or apoptosis of neoplasms or any other mechanism, or that can be otherwise used, in a pharmaceutically-effective amount, to reduce, prevent, mitigate, limit, and/or delay the growth of neoplasms or metastases in a subject with neoplastic disease (e.g. cancer).

Chemotherapeutic agents include, but are not limited to, fluoropyrimidines; pyrimidine nucleosides; purine nucleosides; anti-folates, platinum agents; anthracyclines/anthracenediones; epipodophyllotoxins; camptothecins (e.g., Karenitecin); hormones; hormonal complexes; antihormonals; enzymes, proteins, peptides and polyclonal and/or monoclonal antibodies; immunological agents; vinca alkaloids; taxanes; epothilones; antimicrotubule agents; alkylating agents; antimetabolites; topoisomerase inhibitors; antivirals; and various other cytotoxic and cytostatic agents.

According to a specific embodiment, the chemotherapeutic agent includes, but is not limited to, abarelix, aldesleukin, aldesleukin, alemtuzumab, alitretinoin, allopurinol, altretamine, amifostine, anastrozole, arsenic trioxide, asparaginase, azacitidine, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukindiftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumabozogamicin, goserelin acetate, histrelin acetate, hydroxyurea, IbritumomabTiuxetan, idarubicin, ifosfamide, imatinibmesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolonephenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycinmithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid.

According to one embodiment, the effect of the chemotherapeutic agent is enhanced by about 5%, 10%, 20%, 30%, 40%, 50%, 60%, 70%, 80%, 90%, 95%, 99% or by 100% as compared to the effect of a chemotherapeutic agent in a subject not treated by the DNA editing agent designed to confer a silencing activity and/or specificity of a non-coding RNA molecule or RNA silencing molecule towards a target RNA of interest or second target RNA.

Assessing the efficacy and/or specificity of a chemotherapeutic agent may be carried out using any method known in the art, such as by assessing the tumor growth or the number of neoplasms or metastases, e.g. by MRI, CT, PET-CT, by blood tests, ultrasound, x-ray, etc.

According to one embodiment, the method is affected by targeting non-coding RNA molecules or RNA silencing molecules in healthy immune cells, such as white blood cells e.g. T cells, B cells or NK cells (e.g. from a patient or from a cell donor) to target a RNA of interest or the second target RNA such that the immune cells are capable of decreasing resistance of the cancer to chemotherapy.

According to one embodiment, the method is affected by targeting non-coding RNA molecules or RNA silencing molecules in healthy immune cells, such as white blood cells e.g. T cells, B cells or NK cells (e.g. from a patient or from a cell donor) to target a RNA of interest or the second target RNA such that the immune cells are resistant to chemotherapy.

According to one embodiment, in order to enhance efficacy and/or specificity of a chemotherapeutic agent in a subject, the non-coding RNA molecule or RNA silencing molecule is designed to target a RNA of interest or second target RNA associated with suppression of efficacy and/or specificity of the chemotherapeutic agent.

According to one embodiment, the target RNA of interest or the second target RNA comprises a product of a drug-metabolising enzyme gene (e.g. cytochrome P450 [CYP] 2C8, CYP2C9, CYP2C19, CYP2D6, CYP3A4, CYP3A5, dihydropyrimidine dehydrogenase, uridine diphosphate glucuronosyltransferase [UGT] 1A1, glutathione S-transferase, sulfotransferase [SULT] 1A1, N-acetyltransferase [NAT], thiopurine methyltransferase [TPMT]) and drug transporters (P-glycoprotein [multidrug resistance 1], multidrug resistance protein 2 [MRP2], breast cancer resistance protein [BCRP]).

According to one embodiment, the target RNA of interest or the second target RNA comprises an anti-apoptotic gene. Exemplary target genes include, but are not limited to, Bcl-2 family members, e.g. Bcl-x, IAPs, Flip, Faim3 and SMS1.

According to one aspect of the invention, there is provided a method of inducing cell apoptosis in a subject in need thereof, the method comprising modifying a gene encoding or processed into a non-coding RNA molecule or into an RNA silencing molecule according to the method of some embodiments of the invention, wherein the non-coding RNA molecule or RNA silencing molecule comprises a silencing activity towards a transcript of a gene associated with apoptosis.

According to one aspect of the invention, there is provided a method of inducing cell apoptosis in a subject in need thereof, the method comprising modifying a gene encoding or processed into a non-coding RNA molecule or into an RNA silencing molecule according to the method of some embodiments of the invention, wherein the target RNA of interest or the second target RNA is a transcript of a housekeeping gene, a dominant gene, a gene comprising a high copy number, and/or a gene associated with cell apoptosis, associated with an onset or progression of the disease.

The term “cell apoptosis” as used herein refers to the cell process of programmed cell death. Apoptosis characterized by distinct morphologic alterations in the cytoplasm and nucleus, chromatin cleavage at regularly spaced sites, and endonucleolytic cleavage of genomic DNA at internucleosomal sites. These changes include blebbing, cell shrinkage, nuclear fragmentation, chromatin condensation, and chromosomal DNA fragmentation.

According to one embodiment, cell apoptosis is enhanced by about 5%, 10%, 20%, 30%, 40%, 50%, 60%, 70%, 80%, 90%, 95%, 99% or by 100% as compared to cell apoptosis in a subject not treated by the DNA editing agent conferring a silencing activity and/or specificity of a non-coding RNA molecule or RNA silencing molecule towards a target RNA of interest or second target RNA.

Assessing cell apoptosis may be carried out using any method known in the art, e.g. cell proliferation assay, FACS analysis etc.

According to one embodiment, in order to induce cell apoptosis in a subject, the non-coding RNA molecule or RNA silencing molecule is designed to target a RNA of interest or second target RNA associated with the apoptosis.

According to one embodiment, the target RNA of interest or the second target RNA comprises a product of a BCL-2 family of apoptosis-related genes.

According to one embodiment, the target RNA of interest or the second target RNA comprises an anti-apoptotic gene. Exemplary genes include, but are not limited to, mutant dominant negative TP53, Bcl-x, IAPs, Flip, Faim3 and SMS1.

According to one aspect of the invention, there is provided a method of generating a eukaryotic non-human organism, wherein at least some of the cells of the eukaryotic non-human organism comprise a genome comprising a polynucleotide sequence encoding a non-coding RNA molecule or a silencing molecule having a nucleic acid sequence alteration which results in reduced expression of a housekeeping gene, a dominant gene, a gene comprising a high copy number, and/or a gene associated with cell apoptosis.

According to one embodiment, the reduced expression is by about 10-25%, 10-50%, 10-99%, 20-90%, 25-75%, 30-80%, 40-50%, 50-60%, 60-70%, 70-80% or 90-99% as compared to a eukaryotic non-human organism not generated by the methods of some embodiments of the invention (e.g. wild type organism of the same species).

According to one embodiment, the reduced expression is by about 5%, 10%, 20%, 30%, 40%, 50%, 60%, 70%, 80%, 90%, 95% or 99%, as compared to a eukaryotic non-human organism not generated by the methods of some embodiments of the invention (e.g. wild type organism of the same species).

The DNA or RNA editing agents and optionally the donor oligos of some embodiments of the invention (or expression vectors or RNP complex comprising same) can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the DNA editing agents and optionally the donor oligos accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinylpyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. DNA editing agent) effective to prevent, alleviate or ameliorate symptoms of a disorder (e.g., cancer or infectious disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Animal models for cancerous diseases are described e.g. in Yee et al., Cancer Growth Metastasis. (2015) 8(Suppl 1): 115-118. Animal models for infectious diseases are described e.g. in Shevach, Current Protocols in Immunology, Published Online: 1 Apr. 2011, DOI: 10.1002/0471142735.im1900s93.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NOs: 1-4 are expressed in a DNA sequence format (e.g., reciting T for thymine), but it can refer to either a DNA sequence that corresponds to an gRNA nucleic acid sequence, or the RNA sequence of a RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Cell Culture

Tissue culture is carried out on human cell lines or in mouse embryonic stem cells. Human Bone Osteosarcoma Epithelial Cells (U2OS), Human retinal pigment epithelial cells (RPE1), Adenocarcinomic human alveolar basal epithelial cells (A549), Cervical cancer cells (HeLa) or human colorectal cancer cells (HCT116) are cultured in tissue culture medium supplemented with essential nutrients (amino acids, carbohydrates, vitamins, minerals), growth factors, hormones as needed. The cells are cultured in a CO₂ humidified incubator with controlled temperature (37° C.) under the appropriate physio-chemical conditions (pH buffer, osmotic pressure).

Survival Assay

Chemo-sensitivity is determined by crystal violet assay as previously described [Taniguchi et al., Cell (2002) 109: 459-72]. Cells are seeded onto 12-well plates at 2×10⁴/well and treated with cisplatin, camptothecin (Sigma), paclitaxel (Sigma), AZD2281 (Axon Medchem) or Nutlin3 (Selleckchem) at indicated doses. After incubation for 3 days, monolayers are fixed in 10% methanol containing 10% acetic acid. Adherent cells are stained with 0.5% crystal violet in methanol. The absorbed dye is resolubilized with methanol containing 0.1% SDS, which is transferred into 96-well plates and measured photometrically (595 nm) in a microplate reader. Cell survival is calculated by normalizing the absorbance to that of non-treated controls.

The same method as above can be scaled up to a 6 well plate format or larger and then forming colonies are counted without resolubilizing the crystal violet, this format is called clonogenic assay and is based on the ability of the treated cells to grow into colony. Another assay that is used is the metabolic activity-based cell viability assay XTT or any other metabolic viability assay. XTT is a colorimetric assay used to assess cell viability as a function of cell number based on metabolic activity. This rapid, sensitive, non-radioactive assay is detected using standard microplate absorbance readers. Cells are grown in a 96-well plate at a density of 10⁴-10⁵ cells/well in 100 μL of culture medium with compounds to be tested and are cultured in a CO₂ incubator for 24-48 hours. Fresh buffers are prepared each time before the assay: 10 mM PMS solution in phosphate-buffered saline is and 4 mg of XTT is dissolved in 4 mL of 37° C. cell culture medium. 10 μL of the PMS solution is added to 4 mL of XTT solution immediately before labeling cells. 25 μL of XTT/PMS solution are added directly to each well containing 100 μL cell culture for 2 hours incubation at 37° C. in a CO₂ incubator and absorbance measurements are taking at 450 nm.

Small RNA and miRNA Isolation

Small RNAs including miRNAs are isolated using the miRvana RNA isolation kit (Ambion, Austin, Tex., USA) following the manufacturer's protocol. RNA is quantified using Qubit or Nanodrop spectrophotometer (Thermo Fisher, Wilmington, Del., USA) and quality is determined by Agilent 6000 nanochip (Agilent Technologies, Palo Alto, Calif., USA).

miRNA Measurement

Quantitative Real-Time PCR Analysis is carried out by as follows: RNAs are reverse transcribed and PCR amplified with miScript reverse transcription kit and miScript SYBR PCR kit (Qiagen, Valencia, Calif., USA) using ABI 7500 real-time PCR system following the manufacturer's protocols. Values from duplicate reactions are averaged and normalized to the level of U6 SnoRNA. Relative expression levels are calculated following comparative Ct method as previously described [Schmittgen and Livak. Nat Protoc (2008) 3: 1101-1108]. Alternatively, miRNAs are detected and relatively quantified using small RNA sequence analysis [as described in www(dot)illumina(dot)com/techniques/sequencing/rna-sequencing/small-ma-seq(dot)html or Wake et al., BMC Genomics (2016) 17(1): 1].

Computational Pipeline to Generate GEiGS Templates

The computational Genome Editing induced Gene Silencing (GEiGS) pipeline applies biological metadata and enables an automatic generation of GEiGS DNA templates that are used to minimally edit non-coding RNA genes (e.g. miRNA genes), leading to a new gain of function, i.e. redirection of their silencing capacity to target sequence of interest.

As illustrated in FIG. 1, the pipeline starts with submitting an input: a) target sequence to be silenced by GEiGS; b) the host organism to be gene edited and to express the GEiGS; c) one can choose whether the GEiGS would be expressed ubiquitously or not. If specific GEiGS expression is required, one can choose from a few options (expression specific to a certain tissue, developmental stage, stress, heat/cold shock, etc.).

When all the required input is submitted, the computational process begins with searching among miRNA datasets (e.g. small RNA sequencing, microarray etc.) and filtering (i.e. retaining) only relevant miRNAs that match the input criteria. Next, the selected mature miRNA sequences are aligned against the target sequence and miRNA with the highest complementary levels are filtered. These naturally target-complementary mature miRNA sequences are then modified to perfectly match the target's sequence. Then, the modified mature miRNA sequences are run through an algorithm that predicts siRNA potency and the top 20 with the highest silencing score are filtered. These final modified miRNA genes are then used to generate 200-500 nt ssDNA or 250-5000 nt dsDNA sequences as follows:

200-500 nt ssDNA oligos and 250-5000 nt dsDNA fragments are designed based on the genomic DNA sequence that flanks the modified miRNA. The pre-miRNA sequence is located in the center of the oligo. The modified miRNA's guide strand (silencing) sequence is 100% complementary to the target. However, the sequence of the modified passenger miRNA strand is further modified to preserve the original (unmodified) miRNA structure, keeping the same base pairing profile.

Next, differential sgRNAs are designed to specifically target the original unmodified miRNA gene, and not the modified swapping version. Finally, comparative restriction enzyme site analysis is performed between the modified and the original miRNA gene and differential restriction sites are summarized.

Therefore, the pipeline output includes:
a) 200-500 nt ssDNA oligo or 250-5000 nt dsDNA fragment sequence with minimally modified miRNA
b) 2-3 differential sgRNAs that target specifically the original miRNA gene and not the modified
c) List of differential restriction enzyme sites among the modified and original miRNA gene

Selection of GEiGS Precursors:

A list of non-coding RNA types that are both Dicer substrates and are processed into small silencing RNA was manually curated from the results previously published in Rybak-Wolf A. et al. [Rybak-Wolf A. et al., Cell (2014) 159, 1153,Äì1167] where the PAR-CLIP technique was used to identify RNA molecules bound by Dicer and Argonaute 2 and 3. Dicer substrates were further filtered to exclude regions overlapping with coding genes, and further curated to remove ambiguous annotations. AGO2 and AGO3 smallRNA sequences were processed with cutadapt v1.7 [Martin M., EMBnet.journal (2011) 17(1):10-12] for removing the sequencing adapters. Processed reads where then aligned to GRCh37 assembly of the Human genome using STAR v2.6.1a [Dobin A. et al., Bioinformatics (2013) 29, 15,Äì21] with parameters “--alignIntronMax 1 --alignEndsType EndToEnd --scoreDelOpen -10000 --scoreInsOpen -10000”. Graphics were captured using the Integrated Genomics Viewer software [Thorvaldsdóttir H. et al., Brief Bioinform (2013) 14(2):178-92].

Target Genes

miRNAs with ubiquitous expression profile are chosen (depends on the application, one might choose miRNAs with expression profile that is specific to a certain tissue, developmental stage, temperature, stress, etc.).

For example, miRNAs are modified into siRNA targeting the GFP, p53, BAX, PUMA, NOXA genes (see Table 1, below).

TABLE 1
Target Genes

| | Query sequence | Query sequence |
|---|---|---|
| Gene name | ID | organism |
| P53 | AB082923 | U2OS cells |
| | (SEQ ID NO: 7) | |
| BAX | NM_001291428 | U2OS cells |
| | (SEQ ID NO: 8) | |
| eGFP | AFA52654 | Aequorea victoria |
| | (SEQ ID NO: 12) | |
| PUMA | NM_001127240 | |
| | (SEQ ID NO: 9) | |
| NOXA | NM_021127 | |
| | (SEQ ID NO: 10) | |
| FAS1 | NM_000043 | |
| | (SEQ ID NO: 11) | |

siRNA Design

Target-specific siRNAs are designed by publically available siRNA-designers such as ThermoFisher Scientific's “BLOCK-iT™ RNAi Designer” and Invivogen's “Find siRNA sequences”.

sgRNAs Design

sgRNAs are designed to target the endogenous miRNA genes using the publically available sgRNA designer, as previously described in Park et al. Bioinformatics, (2015) 31(24): 4014-4016. Two sgRNAs are designed for each cassette, and a single sgRNA is expressed per cell, to initiate gene swapping. sgRNAs correspond to the pre-miRNA sequence that is modified post swapping.

In order to maximize the chance of efficient sgRNA choice, two different publicly available algorithms (CRISPER Design: www(dot)crispr(dot)mit(dot)edu:8079/ and CHOPCHOP: www(dot)chopchop(dot)cbu(dot)uib(dot)no/) are used and the top scoring sgRNA from each algorithm is selected.

Swapping ssDNA Oligo Design

400 b ssDNA oligo is designed based on the genomic DNA sequence of the miRNA gene. The pre-miRNA sequence is located in the center of the oligo. Next, the double stranded siRNA sequences are swapped with the mature miRNA sequences in a way that the guide (silencing) siRNA strand is kept 100% complementary to the target. The sequence of the passenger siRNA strand is modified to preserve the original miRNA structure, keeping the same base pairing profile.

Swapping Plasmid DNA Design

4000 bp dsDNA fragment is designed based on the genomic DNA sequence of the miRNA gene. The pre-miRNA sequence is located in the center of the dsDNA fragment. The fragment is cloned into a standard vector (e.g. Bluescript) and transfected into the cells with the Cas9 system components. Next, the mature miRNA sequences are swapped with the double stranded siRNA sequences in a way that the guide (silencing) siRNA strand is kept 100% complementary to the target. The sequence of the passenger siRNA strand is modified to preserve the original miRNA structure, keeping the same base pairing profile.

sgRNAs sequences:
Human miR-150
1.
(SEQ ID NO: 5)
CCAGCACTGGTACAAGGGTTGGG
2.
(SEQ ID NO: 6)
CCAACCCTTGTACCAGTGCTGGG
List of endogenous miRNA that are swapped:
1.
(SEQ ID NO: 13)
Human miR-150
2.
(SEQ ID NO: 14)
Human miR-210
3.
(SEQ ID NO: 19-21)
Human miR-34
5.
(SEQ ID NO: 15)
Human Let7b
6.
(SEQ ID NO: 16)
Human miR-184
7.
(SEQ ID NO: 17)
Human miR-204
8.
(SEQ ID NO: 18)
Human miR-25
ssDNA Oligos used for gene swapping:
Oligo-1:
(SEQ ID NO: 1)
GFP-siRNA1_hsa-mir150 (5′ → 3′)
Oligo-2:
(SEQ ID NO: 2)
GFP-siRNA6_hsa-mir150 (5′ → 3′)
Oligo-3:
(SEQ ID NO: 3)
TP53-siRNA1_hsa-mir150
Oligo-4:
(SEQ ID NO: 4)
TP53-siRNA2 hsa-mir150 (5′ → 3′)
Oligo-5:
(SEQ ID NO: 243)
TP53-siRNA1-mMIR17 (5′ → 3′)
Oligo-6:
(SEQ ID NO: 244)
TP53-siRNA2-mMIR17 (5′ → 3′)
Oligo-7:
(SEQ ID NO: 245)
HPRT-siRNA1-mMIR17 (5′ → 3′)
Oligo-8:
(SEQ ID NO: 246)
HPRT-siRNA2-mMIR17 (5′ → 3′)
Oligo-9:
(SEQ ID NO: 247)
TP53-siRNA1-mMIR21a (5′ → 3′)
Oligo-10:
(SEQ ID NO: 248)
TP53-siRNA2-mMIR21a (5′ → 3′)
Oligo11:
(SEQ ID NO: 249)
HPRT-siRNA1-mMIR21a (5′ → 3′)
Oligo12:
(SEQ ID NO: 250)
HPRT-siRNA2-mMIR21a (5′ → 3′)
Oligo13:
(SEQ ID NO: 251)
GFP-siRNA1-mMIR17 (5′ → 3′)
Oligo14:
(SEQ ID NO: 252)
GFP-siRNA1-mMIR21a (5′ → 3′)

sgRNA Cloning

The transfection plasmid utilized is composed of 4 modules comprising of

1) mCherry driven by the CMV promoter terminated by a BGH poly(A)signal termination sequence.

2) Cas9 (human codon-optimized) driven by the EF1a core promoter terminated by BGH poly(A)signal termination sequence.

3) pol III (U6) promoter sgRNA for guide 1.

Plasmid Design

For transient expression, a plasmid containing three transcriptional units is used. The first transcriptional unit contains the EF1a core promoter-driving expression of Cas9 and the BGH poly(A)signal terminator. The next transcriptional unit consists of CMV promoter driving expression of mCherry and the BGH poly(A)signal terminator. The third contains the pol III (U6) promoter expressing sgRNA to target miRNA genes (each vector comprises a single sgRNAs).

Design and Cloning of CRISPR/CAS9 to Target miR-173 and miR-390 and Introducing SWAPs to Target GFP, AtPDS3 and AtADH1

For proof of concept, the present inventors have designed changes in the sequences of mature miR-173 and miR-390, in their genomic context, to target GFP, AtPDS3 or AtADH1 (in plant cells), by producing small RNA that reverse complements target genes. In addition, to maintain the secondary structure of the miRNA precursor transcript, further changes in the pri-miRNA were carried out (Table 2, below). These fragments were cloned into PUC plasmids and named DONORs and the DNA fragments are referred as SWAPs. For sequences for modifying miR-173—SWAP1 and SWAP2 to target GFP, SWAP3 and SWAP4 to target AtPDS3 and SWAP9 and SWAP10 to target AtADH1 (see Table 2, below). For sequences for modifying miR-390—SWAP5 and SWAP6 to target GFP, SWAP7 and SWAP8 to target AtPDS3 and SWAP11 and SWAP12 to target AtADH1 (see Table 2, below).

Guide RNAs targeting miR-173 and miR-390 were introduced into CRISPR/CAS9 vector system in order to generate a DNA cleavage in the desired miRNA loci. These were co-introduced to plants with the DONOR vectors via gene bombardment protocol, to introduce desired modifications through Homologous DNA Repair (HDR). These guide RNAs are specified in Table 2, below.

TABLE 2
Sequences and oligos used in the experiments

| SEQ ID NO: | Aim |
|---|---|
| 29 | miR173 |
| 30 | miR390 |
| 31 | sgRNA sequence used for miR173 targeting in CRISPR/CAS9 system- |
| | GEiGS#4 |
| 32 | sgRNA sequence used for miR173 targeting in CRISPR/CAS9 system- |
| | GEiGS#5 |
| 33 | sgRNA sequence used for miR390 targeting in CRISPR/CAS9 system- |
| | GEiGS#1 |
| 34 | sgRNA sequence used for miR390 targeting in CRISPR/CAS9 system- |
| | GEiGS#3 |
| 35 | mature GEiGS-siRNA targeting GFP- used in SWAP5 (based on miR390) |
| | and in SWAP1 (based on miR173) |
| 36 | Complementary strand of mature GEiGS-siRNA targeting GFP- used in |
| | SWAP5 (based on miR390) and in SWAP1 (based on miR173) |
| 37 | mature GEiGS-siRNA targeting GFP- used in SWAP6 (based on miR390) |
| | and in SWAP2 (based on miR173) |
| 38 | Complementary strand of mature GEiGS-siRNA targeting GFP- used in |
| | SWAP6 (based on miR390) and in SWAP2 (based on miR173) |
| 39 | mature GEiGS-siRNA targeting AtPDS3- used in SWAP7 (based on |
| | miR390) and in SWAP3 (based on miR173) |
| 40 | Complementary strand of mature GEiGS-siRNA targeting AtPDS3- used |
| | in SWAP7 (based on miR390) and in SWAP3 (based on miR173) |
| 41 | mature GEiGS-siRNA targeting AtPDS3- used in SWAP8 (based on |
| | miR390) and in SWAP4 (based on miR173) |
| 42 | Complementary strand of mature GEiGS-siRNA targeting AtPDS3- used |
| | in SWAP8 (based on miR390) and in SWAP4 (based on miR173) |
| 43 | mature GEiGS-siRNA targeting AtADH1- used in SWAP11 (based on |
| | miR390) and in SWAP9 (based on miR173) |
| 44 | Complementary strand of mature GEiGS-siRNA targeting AtADH1- used |
| | in SWAP11 (based on miR390) and in SWAP9 (based on miR173) |
| 45 | mature GEiGS-siRNA targeting AtADH1- used in SWAP12 (based on |
| | miR390) and in SWAP10 (based on miR173) |
| 46 | Complementary strand of mature GEiGS-siRNA targeting AtADH1- used |
| | in SWAP12 (based on miR390) and in SWAP10 (based on miR173) |
| 47 | Primary transcript of miR173 (pri-miR173) |
| 48 | Primary transcript of SWAP1 (used in Donor vector for targeting GFP) |
| 49 | Primary transcript of SWAP2 (used in Donor vector for targeting GFP) |
| 50 | Primary transcript of SWAP3 (used in Donor vector for targeting PDS3) |
| 51 | Primary transcript of SWAP4 (used in Donor vector for targeting PDS3) |
| 52 | Primary transcript of SWAP9 (used in Donor vector for targeting ADH1) |
| 53 | Primary transcript of SWAP10 (used in Donor vector for targeting ADH1) |
| 54 | Primary transcript of miR390 (pri-miR390) |
| 55 | Primary transcript of SWAP5 (used in Donor vector for targeting GFP) |
| 56 | Primary transcript of SWAP6 (used in Donor vector for targeting GFP) |
| 57 | Primary transcript of SWAP7 (used in Donor vector for targeting PDS3) |
| 58 | Primary transcript of SWAP8(used in Donor vector for targeting PDS3) |
| 59 | Primary transcript of SWAP11 (used in Donor vector for targeting ADH1) |
| 60 | Primary transcript of SWAP12 (used in Donor vector for targeting ADH1) |
| 61 | Sequence of miR173 loci |
| 62 | Oligo sequence of SWAP1 (used in Donor vector for modification of |
| | miR173 for targeting GFP) |
| 63 | Oligo sequence of SWAP2 (used in Donor vector for modification of |
| | miR173 for targeting GFP) |
| 64 | Oligo sequence of SWAP3 (used in Donor vector for modification of |
| | miR173 for targeting PDS3) |
| 65 | Oligo sequence of SWAP4 (used in Donor vector for modification of |
| | miR173 for targeting PDS3) |
| 66 | Oligo sequence of SWAP9 (used in Donor vector for modification of |
| | miR173 for targeting ADH1) |
| 67 | Oligo sequence of SWAP10 (used in Donor vector for modification of |
| | miR173 for targeting ADH1) |
| 68 | Oligo sequence of miR390 loci |
| 69 | Oligo sequence of SWAP5 (used in Donor vector for modification of |
| | miR390 for targeting GFP) |
| 70 | Oligo sequence of SWAP6 (used in Donor vector for modification of |
| | miR390 for targeting GFP) |
| 71 | Oligo sequence of SWAP7 (used in Donor vector for modification of |
| | miR390 for targeting PDS3) |
| 72 | Oligo sequence of SWAP8(used in Donor vector for modification of |
| | miR390 for targeting PDS3) |
| 73 | Oligo sequence of SWAP11 (used in Donor vector for modification of |
| | miR390 for targeting ADH1) |
| 74 | Oligo sequence of SWAP12 (used in Donor vector for modification of |
| | miR390 for targeting ADH1) |
| 75 | qRT for housekeeping gene- 18S expression (NC_037304)- Forward |
| | primer |
| 76 | qRT for housekeeping gene- 18S expression (NC_037304)- Reverse |
| | primer |
| 77 | qRT for analysis of PDS3 expression (AT4G14210)- Forward primer |
| 78 | qRT for analysis of PDS3 expression (AT4G14210)- Reverse primer |
| 79 | qRT for analysis of ADH1 expression (AT1G77120)- Forward primer |
| 80 | qRT for analysis of ADH1 expression (AT1G77120)- Reverse primer |
| 81 | Forward primer for internal amplification of miR390 and its modified |
| | versions |
| 82 | Reverse primer for internal amplification of miR390 and its modified |
| | versions |
| 83 | Forward primer for external amplification of miR390 and its modified |
| | versions- primary reaction |
| 84 | Reverse for external amplification of miR390 and its modified versions- |
| | primary reaction |
| 85 | Forward primer for external amplification of miR390 and its modified |
| | versions- nested reaction |
| 86 | Reverse for external amplification of miR390 and its modified versions- |
| | nested reaction |
| 87 | Forward primer for internal amplification of miR173 and its modified |
| | versions |
| 88 | Reverse primer for internal amplification of miR173 and its modified |
| | versions |
| 89 | Forward primer for external amplification of miR173 and its modified |
| | versions- primary reaction |
| 90 | Reverse for external amplification of miR173 and its modified versions- |
| | primary reaction |
| 91 | Forward primer for external amplification of miR173 and its modified |
| | versions- nested reaction |
| 92 | Reverse for external amplification of miR173 and its modified versions- |
| | nested reaction |

Plasmid Transfection

For transfection Lipofectamine® 2000 Transfection Reagent (or any other) is used according to the manufacturer's protocol, in short:

For adherent cells: One day before transfection, 0.5-2×10⁵ cells are plated in 500 μl of growth medium without antibiotics so that cells will be 90-95% confluent at the time of transfection.

For suspension cells: Just prior to preparing complexes, 4-8×10⁵ cells in 500 μl of growth medium are plated without antibiotics.

For each transfection sample, complexes are prepared as follows: a) DNA is diluted in 50 μl of Opti-MEM® I Reduced Serum Medium without serum (or other medium without serum) and is mixed gently. b) Lipofectamine™ 2000 is mixed gently before use, then the appropriate amount is diluted in 50 μl of Opti-MEM® I Medium, and is incubated for 5 minutes at room temperature. It should be noted that proceeding into step c should be effected within 25 minutes. c) After the 5 minute incubation, the diluted DNA is combined with diluted Lipofectamine™ 2000 (total volume=100 μl) is mixed gently and incubated for 20 minutes at room temperature (solution may appear cloudy). It should be noted that the complexes are stable for 6 hours at room temperature. d) 100 μl of the complexes is added to each well containing cells and medium and is mixed gently by rocking the plate back and forth. e) cells are incubated at 37° C. in a CO₂ incubator for 18-48 hours prior to testing for transgene expression. Medium may be changed after 4-6 hours.

FACS Sorting of Fluorescent Protein-Expressing Cells

48 hrs after plasmid/RNA delivery, cells are collected and sorted for fluorescent protein expression (e.g. mCherry) using a flow cytometer in order to enrich for fluorescent protein/editing agent expressing cells as previously described [Chiang et al., Sci Rep (2016) 6: 24356]. This enrichment step allows bypassing antibiotic selection and collection of only cells transiently expressing the fluorescent protein, Cas9 and the sgRNA. These cells can be further tested for editing of the target gene by HR events followed by efficient silencing of the target gene i.e. GFP.

Bombardment and Plant Regeneration

Arabidopsis Root Preparation:

Chlorine gas sterilised Arabidopsis (cv. Col-0) seeds were sown on MS minus sucrose plates and vernalised for three days in the dark at 4° C., followed by germination vertically at 25° C. in constant light. After two weeks, roots were excised into 1 cm root segments and placed on Callus Induction Media (CIM: ½ MS with B5 vitamins, 2% glucose, pH 5.7, 0.8% agar, 2 mg/l IAA, 0.5 mg/12,4-D, 0.05 mg/l kinetin) plates. Following six days incubation in the dark, at 25° C., the root segments were transferred onto filter paper discs and placed onto CIMM plates, (½ MS without vitamins, 2% glucose, 0.4 M mannitol, pH 5.7 and 0.8% agar) for 4-6 hours, in preparation for bombardment.

Bombardment

Plasmid constructs were introduced into the root tissue via the PDS-1000/He Particle Delivery (Bio-Rad; PDS-1000/He System #1652257), several preparative steps, outlined below, were required for this procedure to be carried out.

Gold Stock Preparation

40 mg of 0.6 μm gold (Bio-Rad; Cat: 1652262) was mixed with 1 ml of 100% ethanol, pulse centrifuged to pellet and the ethanol removed. This wash procedure was repeated another two times.

Once washed the pellet was resuspended in 1 ml of sterile distilled water and dispensed into 1.5 ml tubes of 50 μl aliquot working volumes.

Bead Preparation

In short, the following was performed:

A single tube was sufficient gold to bombard 2 plates of Arabidopsis roots, (2 shots per plate), therefore each tube was distributed between 4 1,100 psi Biolistic Rupture disks (Bio-Rad; Cat: 1652329).

Bombardments requiring multiple plates of the same sample, tubes were combined and volumes of DNA and CaCl2/spermidine mixture adjusted accordingly, in order to maintain sample consistency and minimise overall preparations.

The following protocol summarises the process of preparing one tube of gold, these should be adjusted according to number of tubes of gold used.

All subsequent processes were carried out at 4° C. in an Eppendorf thermomixer. Plasmid DNA samples were prepared, each tube comprising 11 μg of DNA added at a concentration of 1000 ng/μl

1) 493 μl ddH2O was added to 1 aliquot (7 μl) of spermidine (Sigma-Aldrich; S0266), giving a final concentration of 0.1 M spermidine. 1250 μl 2.5M CaCl2 was added to the spermidine mixture, vortexed and placed on ice.

2) A tube of pre-prepared gold was placed into the thermomixer, and rotated at a speed of 1400 rpm.

3) 11 μl of DNA was added to the tube, vortexed, and placed back into the rotating thermomixer.

4) To bind, DNA/gold particles, 70 μl of spermidine CaCl₂) mixture was added to each tube (in the thermomixer).

5) The tubes were vigorously vortexed for 15-30 seconds and placed on ice for about 70-80 seconds.

6) The mixture was centrifuged for 1 minute at 7000 rpm, the supernatant was removed and placed on ice.

7) 500 μl 100% ethanol was added to each tube and the pellet was resuspended by pipetting and vortexed.

8) The tubes were centrifuged at 7000 rpm for 1 minute.

9) The supernatant was removed and the pellet resuspended in 50 μl 100% ethanol, and stored on ice.

Macro Carrier Preparation

The following was performed in a laminar flow cabinet:

1) Macro carriers (Bio-Rad; 1652335), stopping screens (Bio-Rad; 1652336), and macro carrier disk holders were sterilised and dried.

2) Macro carriers were placed flatly into the macro carrier disk holders.

3) DNA coated gold mixture was vortexed and spread (5 μl) onto the centre of each Biolistic Rupture disk.

Ethanol was allowed to evaporate.

PDS-1000 (Helium Particle Delivery System)

In short, the following was performed:

The regulator valve of the helium bottle was adjusted to at least 1300 psi incoming pressure. Vacuum was created by pressing vac/vent/hold switch and holding the fire switch for 3 seconds. This ensured helium was bled into the pipework.

1100 psi rupture disks were placed into isopropanol and mixed to remove static.

1) One rupture disk was placed into the disk retaining cap.

2) Microcarrier launch assembly was constructed (with a stopping screen and a gold containing microcarrier).

3) Petri dish Arabidopsis root callus was placed 6 cm below the launch assembly.

4) Vacuum pressure was set to 27 inches of Hg (mercury) and helium valve was opened (at approximately 1100 psi).

5) Vacuum was released; microcarrier launch assembly and the rupture disk retaining cap were removed.

6) Bombardment on the same tissue (i.e. each plate was bombarded 2 times).

7) Bombarded roots were subsequently placed on CIM plates, in the dark, at 25° C., for additional 24 hours.

Co-Bombardments

When bombarding GEiGS plasmids combinations, 5 μg (1000 ng/μl) of the sgRNA plasmid was mixed with 8.5 μg (1000 ng/μl) swap plasmid and 11 μl of this mixture was added to the sample. If bombarding with more GEiGS plasmids at the same time, the concentration ratio of sgRNA plasmids to swap plasmids used was 1:1.7 and 11 μg (1000 ng/μl) of this mixture was added to the sample. If co-bombarding with plasmids not associated with GEiGS swapping, equal ratios were mixed and 11 μg (1000 ng/μl) of the mixture was added to each sample.

Plant Regeneration

For shoot regeneration, modified protocol from Valvekens et al. [Valvekens, D. et al., Proc Natl Acad Sci USA (1988) 85(15): 5536-5540] was carried out. Bombarded roots were placed on Shoot Induction Media (SIM) plates, which included ½ MS with B5 vitamins, 2% glucose, pH 5.7, 0.8% agar, 5 mg/l 2 iP, 0.15 mg/l IAA. Plates were left in 16 hours light at 25° C.-8 hours dark at 23° C. cycles. After 10 days, plates were transferred to MS plates with 3% sucrose, 0.8% agar for a week, then transferred to fresh similar plates. Once plants regenerated, they were excised from the roots and placed on MS plates with 3% sucrose, 0.8% agar, until analysed.

Phenotypic Analysis

As described above, such as by looking at the fluorescence and cell morphology or other phenotypes such as growth rate/inhibition and/or apoptosis that are dependent on the target gene such Nutlin3 resistance in the case of TP53 silencing.

Anti-Viral Assay

The assay is based on cytopathic effect (CPE) commonly used to determine the potency of purified interferon stocks. In the CPE assay, anti-viral activity is measured based on its ability to inhibit virus-induced cytopathology as measured by a crystal violet live-cell stain [previously described by Rubinstein et al., J Virol. (1981) 10:755-758].

VSV forms discrete, microscopic plaques in stationary cultures of the WISH amnion cell line. Microplaque formation is rapid, reproducible, and easily quantitated, occurs at temperatures ranging from 33 to 40° C., and does not require a semisolid overlay.

Allyl Alcohol Selection

For selection of plants with allyl alcohol, 10 days post bombardment, roots were placed on SIM media. Roots were immersed in 30 mM allyl alcohol (Sigma-Aldrich, US) for 2 hours. Then the roots were washed three times with MS media, and placed on MS plates with 3% sucrose, 0.8% agar. Regeneration process was carried on as previously described.

Genotyping

Plant tissue samples were treated and amplicons amplified in accordance to the manufacturers recommendations. MyTaq Plant-PCR Kit (BioLine BIO 25056) for short internal amplification and Phire Plant Direct PCR Kit (Thermo Scientific; F-130WH) for longer external amplifications. Oligos used for these amplifications are specified in Table 2, above. Different modifications in the miRNA loci were identified through different digestion patterns of the amplicons, as follows:

For modifications of miR-390—internal amplicon was 978 base pairs long, and for external amplification it was 2629 base pairs. For the identification of swap 7, digestion with NlaIII resulted in a fragment size of 636 base pairs, while in the wt version it was cleaved to 420 and 216 long fragments. For the identification of swap 8, digestion with Hpy188I resulted in fragments size of 293 and 339 base pairs, while in the wt version this site was absent and resulted in a 632-long fragment. For the identification of swaps 11 and 12, digestion with BccI resulted in a fragment size of 662 base pairs, while in the wt version it was cleaved to 147 and 417 long fragments.

For modifications of miR-173-internal amplicon was 574 base pairs long, and for nested external amplification it was 466 base pairs. For the identification of swap 3, digestion with BslI resulted in fragments size of 217 and 249 base pairs in the external amplicon and 317 and 149 in the internal one. In the wt version this site was absent and resulted in a 466-long fragment in the external amplicon and 574 in the internal reaction. For the identification of swap 4, digestion with BtsαI resulted in fragments size of 212 and 254 base pairs in the external amplicon and 212 and 362 in the internal one. In the wt version, this site was absent and resulted in a 466-long fragment in the external amplicon and 574 in the internal reaction. For the identification of swap 9, digestion with NlaIII resulted in fragments size of 317 and 149 base pairs in the external amplicon and 317 and 244 in the internal one. In the wt version, this site was absent and resulted in a 466-long fragment in the external amplicon and 561 in the internal reaction. For the identification of swap 10, digestion with NlaIII resulted in fragments size of 375 and 91 base pairs in the external amplicon and 375 and 186 in the internal one. In the wt version, this site was absent and resulted in a 466-long fragment in the external amplicon and 561 in the internal reaction.

DNA and RNA Isolation

Plant samples were harvested into liquid nitrogen and stored in −80° C. until processed. Grinding of tissue was carried out in tubes placed in dry ice, using plastic Tissue Grinder Pestles (Axygen, US). Isolation of DNA and total RNA from ground tissue was carried out using RNA/DNA Purification kit (cat. 48700; Norgen Biotek Corp., Canada), according to manufacturer's instructions. In the case of low 260/230 ratio (<1.6), of the RNA fraction, isolated RNA was precipitated overnight in −20° C., with 1 μl glycogen (cat. 10814010; Invitrogen, US) 10% V/V sodium acetate, 3 M pH 5.5 (cat. AM9740, Invitrogen, US) and 3 times the volume of ethanol. The solution was centrifuged for 30 minutes in maximum speed, at 4° C. This was followed by two washes with 70% ethanol, air-drying for 15 minutes and resuspending in Nuclease-free water (cat. 10977035; Invitrogen, US).

Reverse transcription (RT) and quantitative Real-Time PCR (qRT-PCR) One microgram of isolated total RNA was treated with DNase I according to manufacturer's manual (AMPD1; Sigma-Aldrich, US). The sample was reverse transcribed, following the instructor's manual of High-Capacity cDNA Reverse Transcription Kit (cat 4368814; Applied Biosystems, US).

For gene expression, Quantitative Real Time PCR (qRT-PCR) analysis was carried out on CFX96 Touch™ Real-Time PCR Detection System (BioRad, US) and SYBR® Green JumpStart™ Taq ReadyMix™ (S4438, Sigma-Aldrich, US), according to manufacturers' protocols, and analysed with Bio-RadCFX manager program (version 3.1). For the analysis of AtADH1 (AT1G77120) the following primer set was used: Forward GTTGAGAGTGTTGGAGAAGGAG SEQ ID NO: 237 and reverse CTCGGTGTTGATCCTGAGAAG SEQ ID NO: 238; For the analysis of AtPDS3 (AT4G14210), the following primer set was used: Forward GTACTGCTGGTCCTTTGCAG SEQ ID NO: 239 and reverse AGGAGCACTACGGAAGGATG SEQ ID NO: 240; For endogenous calibration gene, the 18S ribosomal RNA gene (NC_037304) was used—Forward ACACCCTGGGAATTGGTTT SEQ ID NO: 241 and reverse GTATGCGCCAATAAGACCAC SEQ ID NO: 242.

### Example 1A

### Genome Editing Induced Gene Silencing (GEiGS) Platform

MicroRNAs (miRNAs) MicroRNAs (miRNAs) are small endogenous non-coding RNAs (ncRNAs) of 20 to 24-nucleotide in length, originating from long self-complementary precursors. Mature miRNAs regulate gene expression in two ways; (i) by inhibiting translation or (ii) by degrading coding mRNAs by perfect or near-perfect complement with the target mRNAs. In animals, seminal studies on miRNAs have shown that only the seed region (sequence spanning from position 2 to 8 at the 5′ end), is crucial for target recognition. The seed sequence pairs fully to its responsive element mainly at the 30-untranslated region (UTR) of the target mRNA. The alteration of miRNAs biogenesis mechanism, miRNAs expression level and miRNAs regulatory networks affects important biological pathways such as cellular differentiation and apoptosis and it is detected in various human diseases and syndromes, especially in cancer.

All tumors present specific signatures of miRNAs altered expression. For this reason, miRNAs expression profiles of tumors may represent valid and useful biomarkers for diagnosis, prognosis, patient stratification, definition of risk groups and monitoring of the response to therapy. Equally relevant is the emerging role of miRNAs in viral infections. Data from literature show a mutual interference between viruses and the host cell's miRNA machinery. For instance, viruses may impair the host cell's miRNA pathway by interacting with specific proteins, synthesize their own miRNAs to modify cellular environment or to regulate their own mRNAs, or make use of cellular miRNAs to their favor. However, it is also true that host cell's miRNAs may target viral mRNAs. In many cases, this bidirectional interference is resolved in favor of the viruses that as a result may escape the immune response and complete the replication cycle.

Accordingly, the present inventors are utilizing endogenous ncRNA sequences (e.g. of miRNA) that are re-designed using GEiGS to gain silencing functionality, by Homologous Recombination (HR), in order to specifically silence any RNA of interest. In order to replace chosen sequences, HR uses longer stretches of sequence homology flanking the DSB site to repair DNA lesions and is therefore considered to be accurate mechanism for DSB repair due to the requirement of higher sequence homology between the damaged and intact donor strands of DNA (i.e. the inserted siRNA sequence). This process is considered to be error-free if the DNA template used for repair is identical to the original DNA sequence at the DSB, or if a template-free methods is utilized, or it can introduce very specific mutations into the damaged DNA e.g. swapping genes.

### Example 1B

### Genome Editing Induced Gene Silencing (GEiGS)

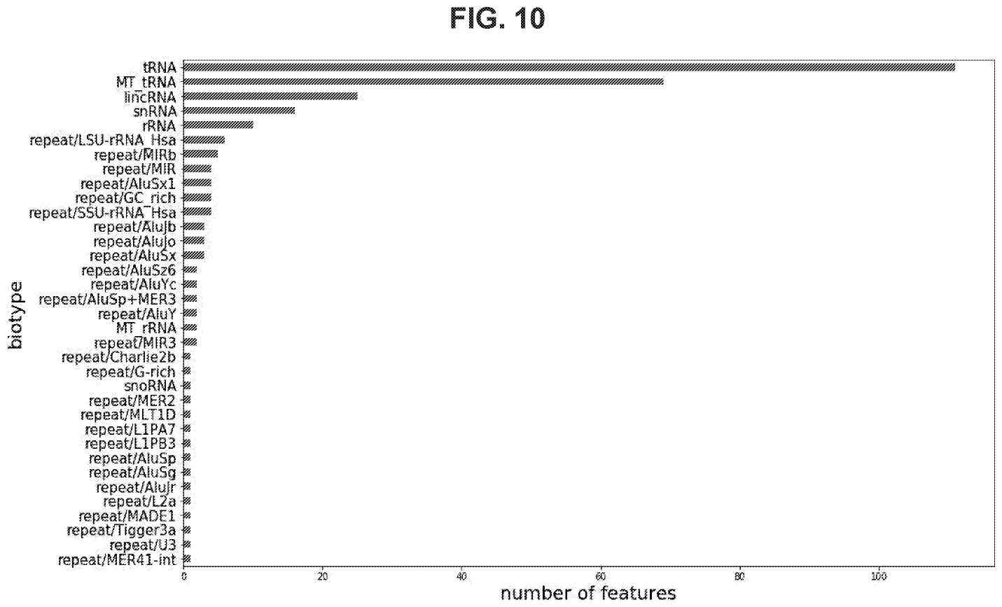

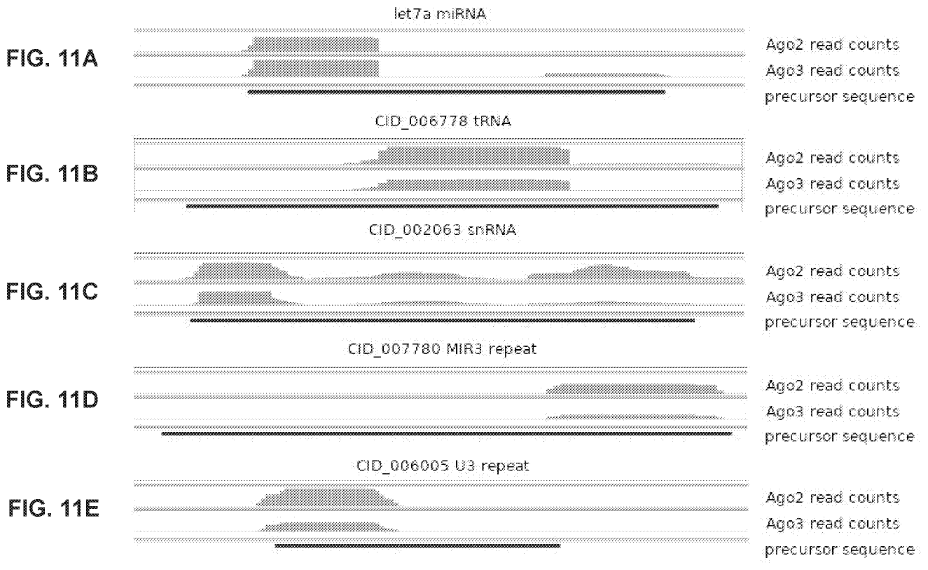

In order to design GEiGS oligos, template non-coding RNA molecules (precursors) that are processed and give raise to derivate small silencing RNA molecules (matures) are required. The present inventors have characterized dicer substrate RNAs (i.e. cellular RNAs that are bound by Dicer) that produce silencing engaged small RNAs (i.e. small RNAs that are bound by Argonaut 2 and Argonaut 3) in human and C. elegans as previously discussed in Rybak-Wolf [Rybak-Wolf, A. et al., Cell (2014) 159: 1153,Äì1167]. Crossing both datasets (dicer bound RNAs & Ago2 and Ago3 bound small RNAs), allowed to generate a list of non-coding RNAs that are precursors of small silencing RNAs (FIG. 10 and FIGS. 11A-E). Two sources of precursor and their corresponding mature sequences were used for generating GEiGS oligos. For miRNAs, sequences were obtained from the miRBase database [Kozomara, A. and Griffiths-Jones, S., Nucleic Acids Res (2014) 42: D68,ÄìD73]. Other type of precursors (including tRNAs, snRNAs, and various types of repeats) were obtained from a recent publication describing Dicer-bound & AGO-bound RNAs [Rybak-Wolf, A. et al., Cell (2014) 159: 1153,Äì1167].

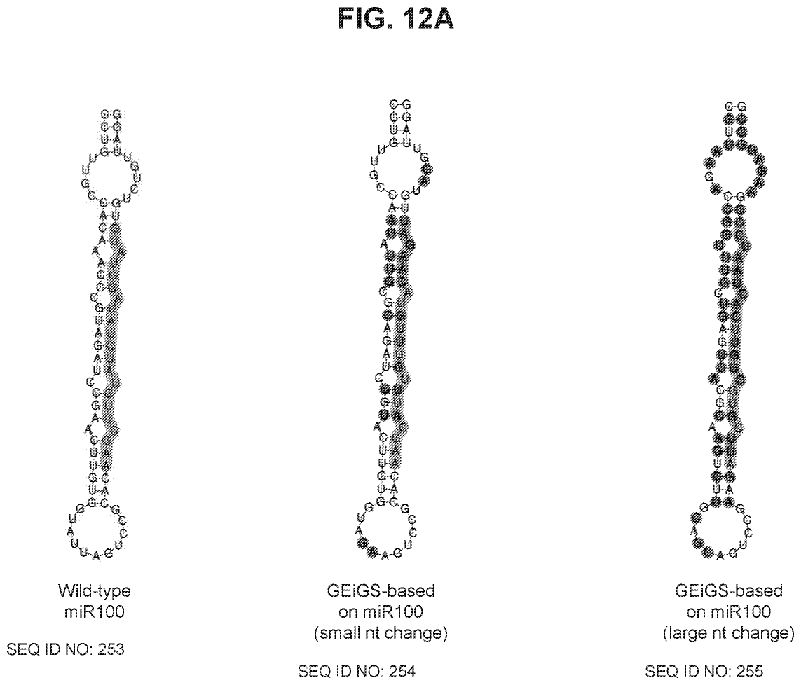

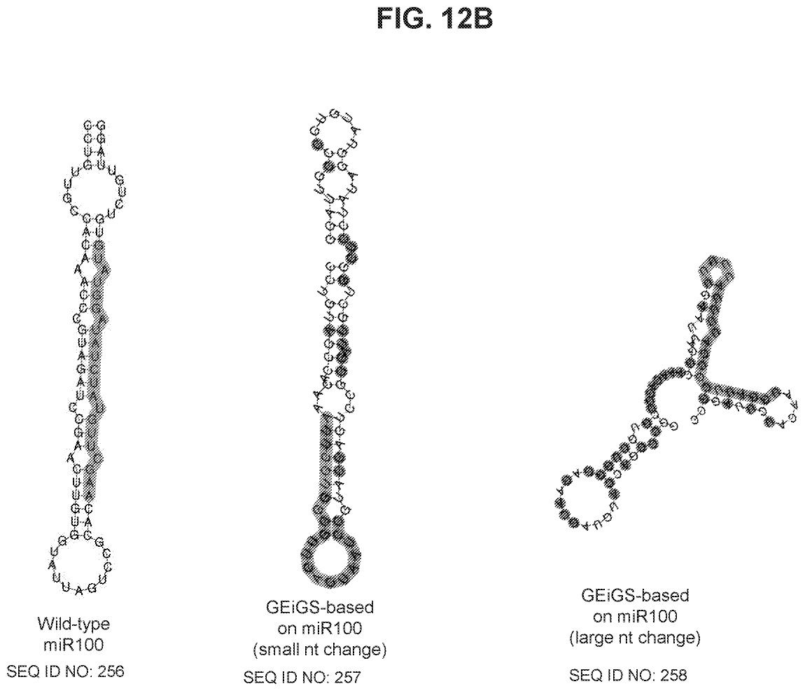

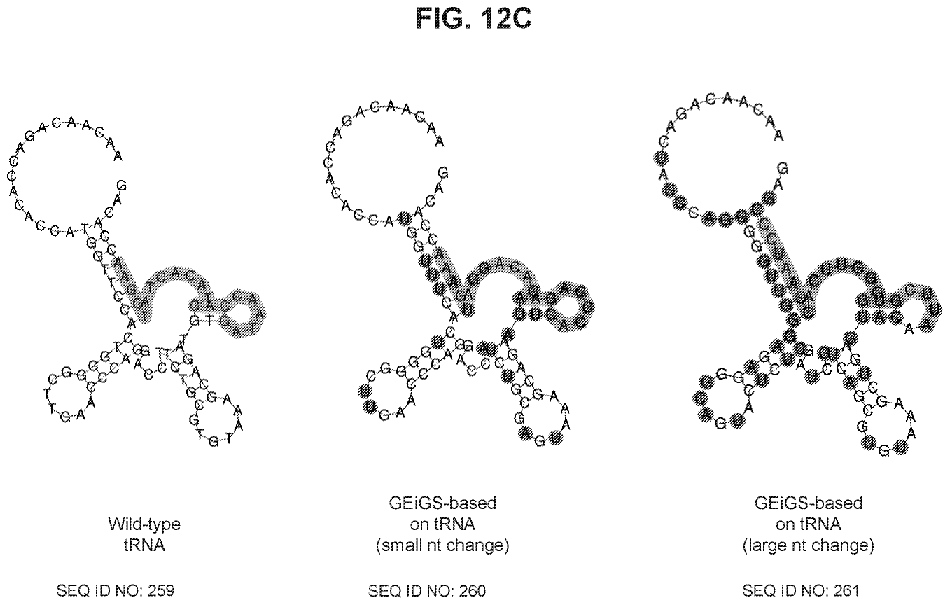

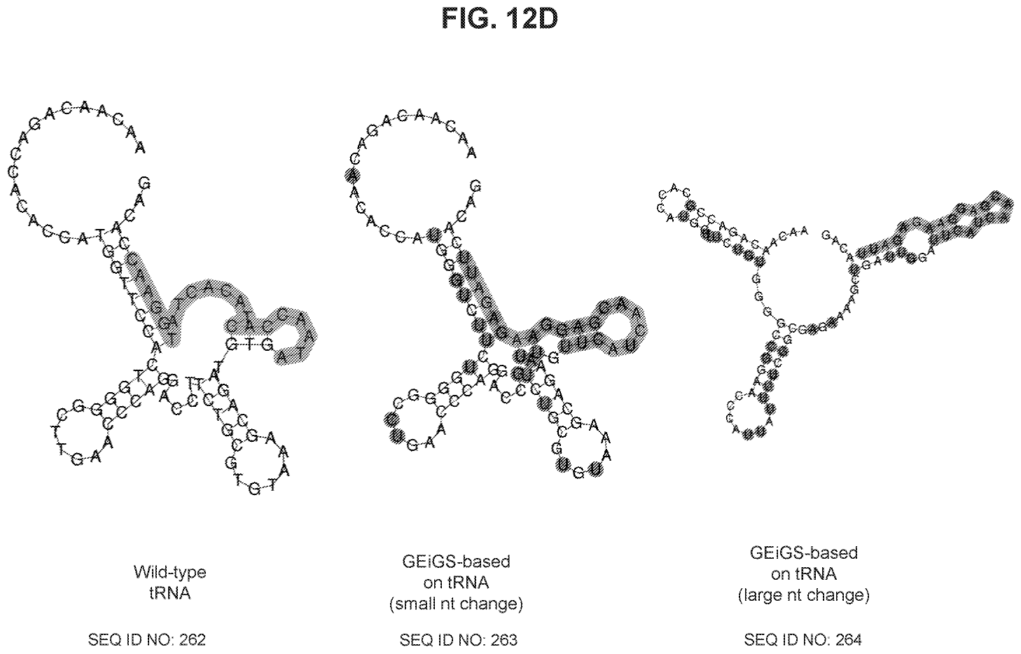

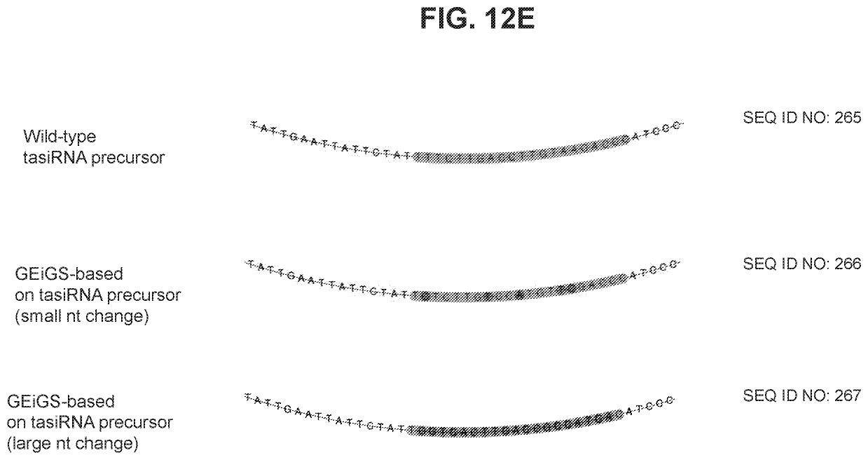

Silencing targets were chosen in a variety of host organisms. siRNAs were designed against these targets using the siRNArules software [Holen, T., RNA (2006) 12: 1620,Äì1625]. Each of these siRNA molecules was used to replace the mature sequences present in each precursor, generating “naive” GEiGS oligos. The structure of these naive sequences was adjusted to approach the structure of the wild type precursor as much as possible using the ViennaRNA Package v2.6 [Lorenz, R. et al., ViennaRNA Package 2.0. Algorithms for Molecular Biology (2011) 6: 26]. Examples for successful structure maintenance versus non-successful structure maintenance can be found in FIG. 12A-D. After the structure adjustment, the number of sequence and secondary structure changes between the wild type and the modified oligo were calculated. These calculations are essential to identify potentially functional GEiGS oligos that require minimal sequence changes with respect to the wild type (FIG. 12A-E).

CRISPR/cas9 small guide RNAs (sgRNAs) against the wild type precursors were generated using the CasOT software [Xiao, A. et al., Bioinformatics (2014) 30: 1180,Äì1182]. sgRNAs were selected where the modifications applied to generate the GEiGS oligo affect the PAM region of the sgRNA, rendering it ineffective against the modified oligo.

### Example 2

### GEiGS of an “Endogenous” Transgene

A quick and robust approach to check the efficiency of GEiGS is to silence a transgene, which will serve as endogenous gene and in addition is also a marker gene like GFP (green fluorescent protein). There are few options to assess the effectiveness of GFP silencing in cells, the present inventors are using FACS analysis, RT-qPCR and microscopy to assess the effectiveness of GFP silencing in cells.

Silencing of GFP is well characterized and there are many available short interfering RNA sequences (siRNA) that are efficient in triggering GFP silencing. Therefore, for gene swapping, the present inventors are using 21 mer siRNA molecules designed to silence GFP. Additionally or alternatively, the present inventors are using public algorithms that predict which siRNA will be effective in initiating gene silencing to a given gene (e.g. GFP). Since the predictions of these algorithms are not 100%, the present inventors are only using sequences that are the outcome of at least two different algorithms.

In order to use siRNA sequences that will silence the GFP gene, the present inventors are swapping them with a known endogenous miRNA gene sequences using the CRISPR/Cas9 system. There are many databases of characterized miRNAs, the present inventors are choosing several known human miRNAs with different expression profiles (e.g. low constitutive expression, highly expressed, induced in stress, etc.). In order to swap the endogenous miRNA sequence with siRNA the present inventors are using the HR approach.

As illustrated in FIG. 2, using HR the present inventors are contemplating two options: 1) use a donor ssDNA oligo sequence of around 200-500 bases which includes the swapping siRNA sequence in the middle or 2) use plasmids expressing 1 Kb-4 Kb insert which is almost 100% identical to the miRNA surrounding in the genome except the 2×21 bp of the miRNA and the *miRNA that is changed into the siRNA of the GFP (500-2000 bp up and downstream the siRNA). The transfection includes a few constructs: CRISPR:Cas9/RFP sensor to track and enriched for positive transformed cells, gRNAs that guide the Cas9 to produce a DSB which is repaired by HR depending on the insertion vector/oligo.

The insertion vector contains two continuous regions of homology surrounding the targeted locus that are replaced (e.g. miRNA) and is modified to carry the mutation of interest (i.e. siRNA). If plasmid is used, the targeting construct is used as a template for homologous recombination ending with the replacement of the miRNA with the siRNA of choice. After transfection to tissue culture cells, FACS is used to enrich for positive Cas9/sgRNA transfected events, cells are scored for GFP silencing under microscope (as illustrated in FIG. 2). It is expected that the positive edited cells will produce siRNA sequences targeting the GFP gene and therefore the GFP expression of the transgene will be silenced compared to control cells.

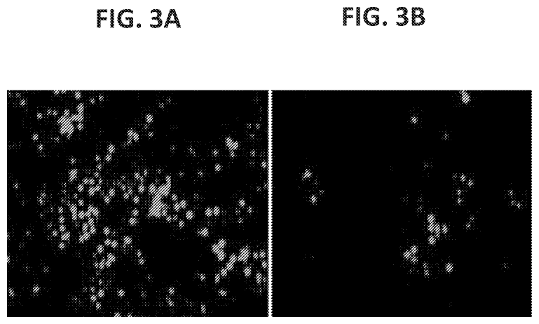

In order to show proof of concept (POC) of GFP silencing using GEiGS, transgenic human cell lines including U2OS, RPE1, A549 or Hela cells that express GFP are being utilized. Cells are transfected with GEiGS methodology and with cassettes to swap endogenous non-coding RNAs (e.g. miRNA) and turn it into a non-coding RNA that is processed into siRNAs targeting GFP to initiate the RNA silencing mechanism against GFP. As illustrated in FIGS. 3A-B, knock down of GFP gene expression levels in human cells results in reduced expression of GFP in cells expressing siGFP (i.e. in which GFP is silenced) as compared to control cells (FIG. 3A).

### Example 3

### GEiGS of Exogenous Transgene (GFP) in Tissue Culture Cells

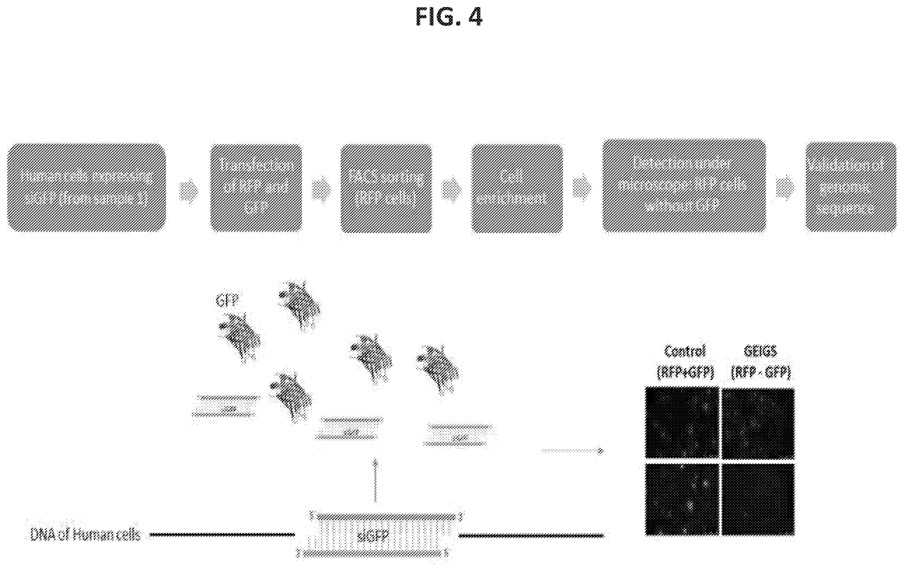

In addition to the former example of GFP silencing (Example 2 above), another way to demonstrate the efficiency of GEiGS is to silence a marker gene like GFP in a transient GFP transfection assay. As illustrated in FIG. 4, human cells are treated using GEiGS in order to redirect silencing specificity of endogenous miRNA through expression of small siRNA molecules targeting the GFP gene (as discussed in Example 2, above). Control untreated cells and GEiGS-GFP cells (i.e. expressing siGFP) are then transfected with a plasmid expressing separately two markers (sensor) GFP+RFP (Red Fluorescent Protein), cells which express only RFP but not GFP in the GEiGS treatment are the results of GFP gene silencing due to siGFP expression. DNA from these cells (Red but lack of GFP expression) are extracted and examined for the correct genome-editing event. Furthermore, the cells can be analyzed for the loss of expression of GFP e.g., by fluorescent detection of GFP or q-PCR, HPLC.

### Example 4

### GEiGS of TP53 or HPRT Expression Inhibits Nutlin3-Induced or 6TG (Thioguanine, 6-TG, 6-Tioguanine) Cell Death/Growth Inhibition in U2OS and RPE1 or Mouse Embryonic Stem (mES) Cells

To show POC of GEiGS in human cells, the present inventors are working with U2OS, RPE1 or mouse embryonic stem cells. U2OS are cells that grow fast and are easy to transfect with high efficiency. These cells originate from bone cancer—osteosarcoma. RPE1 are epithelial cells originated from normal retina (i.e. not from a disease or sick culture) with normal and active TP53 as do mES.

TP53 is a tumor-suppressor protein that induces directly or indirectly apoptotic cell death in response to oncogenic stress. The consequences of DNA damage depend on the cell type and the severity of the damage. Mild DNA damage can be repaired with or without cell-cycle arrest. More severe and irreparable DNA injury leads to the appearance of cells that carry mutations or causes a shift towards induction of the senescence or cell death programs. Although for many years it was argued that DNA damage kills cells via apoptosis or necrosis, technical and methodological progress during the last few years has helped to reveal that this injury might also activate death by autophagy or mitotic catastrophe, which may then be followed by apoptosis or necrosis. The molecular basis underlying the decision-making process is currently the subject of intense investigation.

Today, anyone with interest in cancer research is already well aware of the existence of TP53 and its relevance to practically every aspect of tumor biology. TP53 is undoubtedly one of the most extensively studied genes and proteins. Early studies indicate that transactivation-defective mutants of p53 are capable of inducing apoptosis, implying a transcription-independent role for p53 in apoptosis. DNA-damage leads to mitochondrial translocation of TP53. TP53 binds to Bcl-2 family protein Bcl-xL to influence cytochrome c release. TP53 directly activates the proapoptotic Bcl-2 protein Bax in the absence of other proteins to permeabilize mitochondria and engage the apoptotic program. TP53 can release both proapoptotic multidomain proteins and BH3-only proteins that are sequestered by Bcl-Xl. In addition, TP53 can directly mediate mitochondrial mechanism of apoptosis by facilitating Bax oligomerization, binding to Bcl-xL, but not to Bax, TP53-Bcl-xL interaction releases Bax and released Bax forms oligomers in mitochondrial membrane, leading cytochrome c release and apoptosis (the proline-rich domain, aa 62-91 in mouse, of TP53 is required for this effect) [Jerry et al. Science (2004) 303(5660):1010-4]. TP53 also act as a transcription factor promoting the expression of the pro-apoptotic genes such as BAX, PUMA and NOXA.

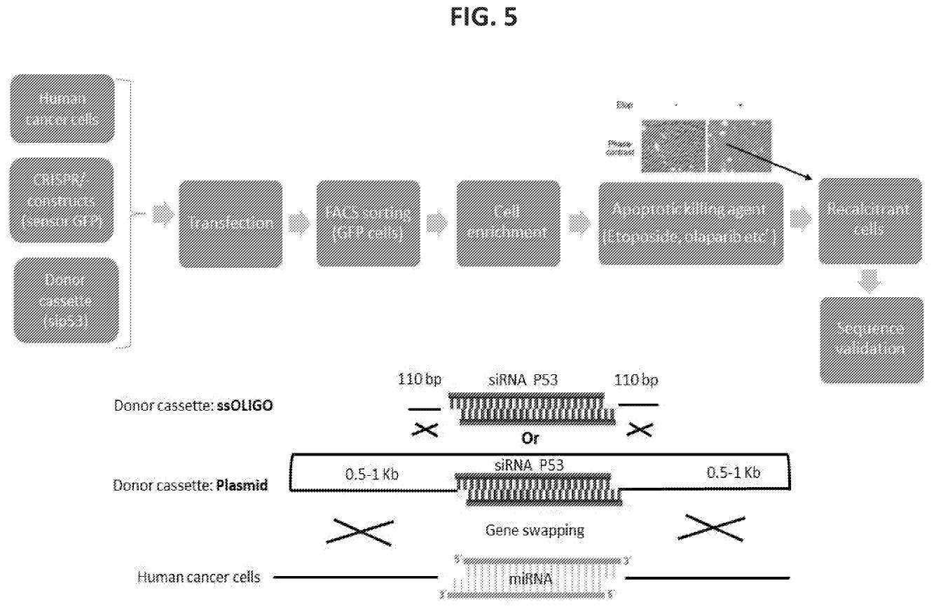

As illustrated in FIG. 5, the present inventors are modifying RPE1 cells to express siRNA directed against TP53, these cells when exposed to Nutlin3 or chemotherapy (e.g. Camptothecin (CPT), etoposide, olaparib, etc.) show inhibition of cell death. One of the assays the present inventors are utilizing is the crystal violet assay in which staining of cells enable to compare cell number (density) and morphology, which differ between healthy and dying cells. Cell clones that are resistant to cell death are verified to the correct genome editing event and for expression of the relevant TP53 siRNA. Furthermore, the cells can be analyzed for the loss of expression of TP53 e.g., by fluorescent detection of GFP or q-PCR, HPLC.

Tioguanine, also known as thioguanine or 6-thioguanine (6-TG) is a medication commonly used to treat acute myeloid leukemia (AML), acute lymphocytic leukemia (ALL), and chronic myeloid leukemia (CML). Tioguanine, an antimetabolite, is a purine analogue of guanine and works by disrupting DNA and RNA. 6-Thioguanine is a thio analogue of the naturally occurring purine base guanine. 6-thioguanine utilises the enzyme hypoxanthine-guanine phosphoribosyltransferase (HGPRTase/HPRT) to be converted to 6-thioguanosine monophosphate (TGMP). High concentrations of TGMP may accumulate intracellularly and hamper the synthesis of guanine nucleotides via the enzyme Inosine monophosphate dehydrogenase (IMP dehydrogenase). TGMP is converted by phosphorylation to thioguanosine diphosphate (TGDP) and thioguanosine triphosphate (TGTP). Simultaneously deoxyribosyl analogs are formed, via the enzyme ribonucleotide reductase. The TGMP, TGDP and TGTP are collectively named 6-thioguanine nucleotides (6-TGN). 6-TGN are cytotoxic to cells by: (1) incorporation into DNA during the synthesis phase (S-phase) of the cell; and (2) through inhibition of the GTP-binding protein (G protein) Rac1, which regulates the Rac/Vav pathway. An additional effect may be derived from the incorporation of 6-thioguanine into RNA. This yields a modified RNA strand which cannot be read by the ribosomes.

In brief, loss or reduction of HPRT gene expression render the cells resistant to 6TG. Accordingly, the present inventors are modifying HPRT gene expression by expressing siRNA directed against HPRT, and analyzing downregulation of HPRT by resistance to 6TG.

### Example 5

### GEiGS of Pro-Apoptotic Genes (BAX, PUMA, NOXA) Inhibits Chemotherapy-Induced Cell Death in Human Cancer Cells

In this experiment the present inventors are using U2OS cells. In order to create cells resistant to chemotherapy agents like CPT, etoposide, olaparib, etc., the present inventors are first using siRNA capable of targeting apoptotic genes like BAX, PUMA and NOXA which are known as pro-apoptotic genes.

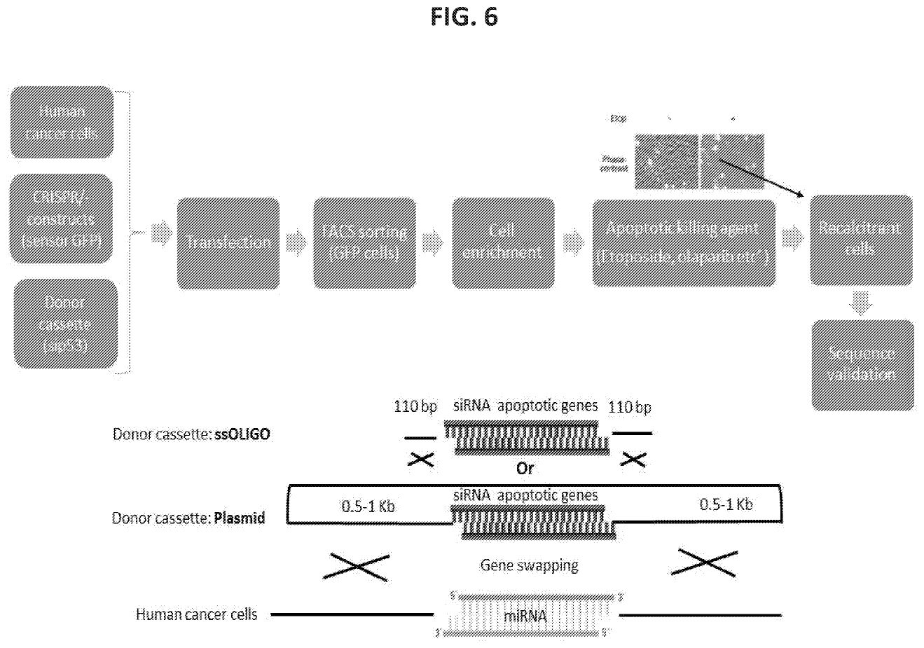

As illustrated in FIG. 6, the present inventors are treating U2OS cells using GEiGS to express siRNA targeting apoptotic genes. Modified cells that express siRNA are expected to be resistant to chemotherapy (e.g. like CPT, etoposide, olaparib, etc.)-induced cell death. After transfection with GEiGS cassettes+RFP sensor, transfected cells are enriched with FACS and cells are exposed to chemotherapy agents. In the control, all cells are sensitive and die or enter senescence (easy to detect under a microscope using Dapi staining, few cells with big nuclei). Clones that are resistant to cell death and or senescence are expected to be positively expressing edited siRNAs and are verified to the have the correct genome editing modification and expression of the relevant siRNA. Furthermore, the cells can be analyzed for the loss of expression of apoptotic genes like BAX, PUMA and NOXA e.g., by fluorescent detection of GFP or q-PCR, HPLC.

### Example 6

### Utilizing GEiGS to Immunize Human Cells Against Viral Infection

In order to prove that GEiGS is a robust method for human immunization with the ability to knock down exogenous pathogenic gene, the present inventors are providing an example of silencing of a virus gene. A lentiviral system is very effective at delivering genetic material to whole model organisms and almost all mammalian cells, including non-dividing non growing cells, as well as difficult-to-transfect cells including neuron, primary and stem cells. The efficiency of lentiviral transduction is close to 100% depending on the Multiplicity Of Infection (MOI), making it ideal as an expression vector system.

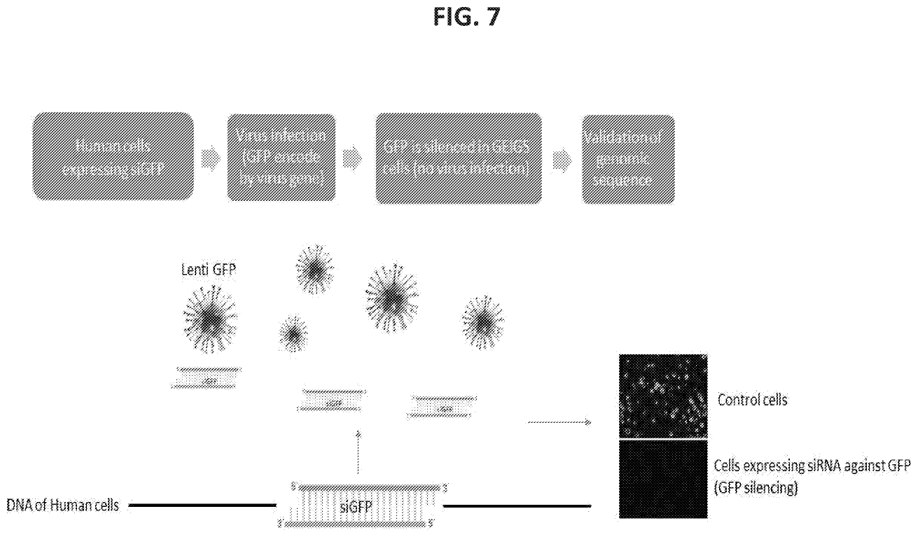

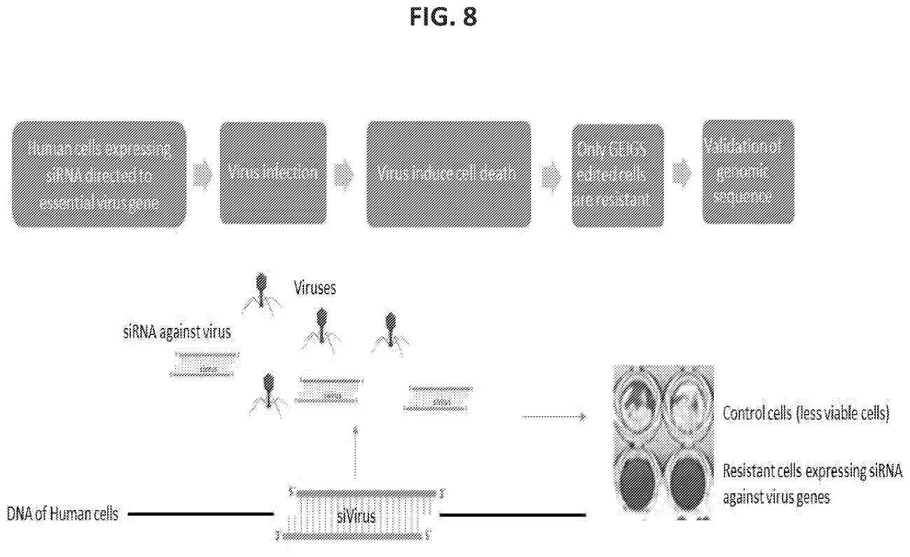

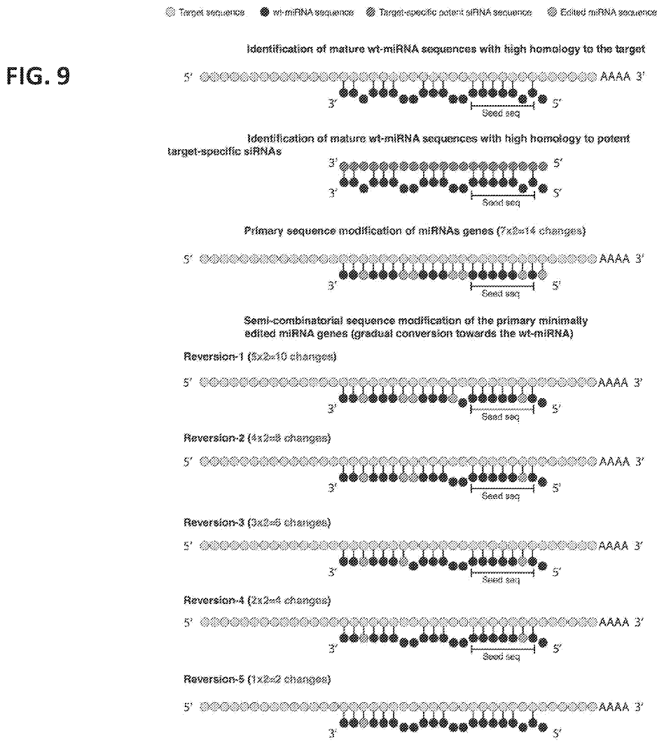

Control cells that are infected with lentivirus expressing-GFP show expression of GFP under the microscope (as illustrated in FIG. 7). GEiGS-GFP cells engineered to express siRNA targeting GFP gene (as illustrated in Example 2, above) are expected to show reduced levels of GFP (as illustrated in FIG. 7). Generating GEiGS cells with no or low GFP gene expression after infection with Virus-GFP (e.g. Lenti-GFP) will prove that silencing of exogenous gene was achieved and that GEiGS is an effective method to immunize human cells against invasive infectious RNA like viruses.

There are few easy options to assess the effectiveness of the GFP gene silencing in the cell, the present inventors are using FACS analysis, RT-qPCR, microscopy and/or immunoblotting. Therefore, for gene swapping, the present inventors designed 21 mer siRNA molecules (as described in Example 2, above). The present inventors are using public algorithms that predict which siRNA will be effective in initiating gene silencing to a given gene (as described in Example 2, above).

### Example 7

### Immunizing Human Cells to Virus Infection by Silencing of an Exogenous Virus Gene (Cell Survival Assay)

In order to prove that GEiGS is a robust method for human immunization with the ability to knock down exogenous genes, in addition to example using lentivirus expressing GFP (Example 6, above), the present inventors are using wild-type RNA virus infection and are scoring for cell survival. The present inventors are providing an example of silencing of a Vesicular stomatitis virus (VSV) gene.

VSV, a Rhabdoviridae RNA virus, can infect many cell types and therefore is a common laboratory virus used to study the properties of viruses in the family Rhabdoviridae, as well as to study viral evolution. VSV is an arbovirus, and its replication occurs in the cytoplasm. The genome of VSV is on a single molecule of negative-sense RNA that has 11,161 nucleotides in length that encodes five major proteins: G protein (G), large protein (L), phosphoprotein, matrix protein (M) and nucleoprotein. In healthy human cells, the virus cannot reproduce (probably because of the interferon response) but in many cancer cells (that have a reduced interferon response) VSV can grow and hence lyse the oncogenic cells. A functional anti-viral assay based on cytopathic effect (CPE) is utilized to determine cell survival as described in detail in the ‘general materials and experimental procedures’ section above. This method allows evaluating and comparing cell survival and viability. Through staining cells it is possible to compare cell number, density and morphology, which differ between healthy and dying cells.

In order to find efficient siRNAs targeting VSV genes, a preliminary experiment with different transfection of siRNAs targeting virus genes is carried out. siRNAs that inhibit VSV-induced cell death are used with GEiGS to edit human WISH cells to express these siRNAs. Control cells that are infected with VSV will show cytopathology effect as measured by a crystal violet compared to GEiGS cells that are expected to be resistant to virus infection.

### Example 8

### GEiGS of the Pro-Apoptotic FAS Gene Expression Reduces 5-Fluorouracil-Induced Apoptosis in HCT116 Cells

It was previously shown by Pedro et al. [Pedro et al. Biochimica et Biophysica Acta (2007) 1772: 40-47] that in HCT116 human colorectal cancer cells expressing wild-type p53, silencing of FAS expression by RNA interference moderates 5-FU-induced apoptosis.

HCT116 cells are treated using GEiGS to express siRNA targeting FAS gene. HCT116 control and GEiGS positive cells (expressing FAS siRNA) are treated with 5-FU (e.g. 1-8 μM) for e.g. 8-48 hours. Cell viability is evaluated by XTT and trypan blue dye exclusion. Apoptosis is assessed by changes in nuclear morphology and caspase 3 activity. 5-FU is cytotoxic in HCT116 cells but when siRNA is used to inhibit Fas, 5-FU-mediated nuclear fragmentation and caspase 3 activity are expected to be markedly reduced.

### Example 9

### Generation of Plants with Modified Endogenous miRNA to Target Different Genes

Minimal modifications in the genomic loci of a miRNA, in its recognition sequence (which will mature to a miRNA) can lead to a new system to regulate new genes, in a non-transgenic manner. Therefore, an agrobacterium-free transient expression method was used, to introduce these modifications by bombardment of Arabidopsis roots, and their regeneration for further analysis. The present inventors had chosen to target two genes, PDS3 and ADH1 in Arabidopsis plants.

Carotenoids play an important role in many physiological processes in plants and the phytoene desaturase gene (PDS3) encodes one of the important enzymes in the carotenoid biosynthesis pathway, its silencing produces an albino/bleached phenotype. Accordingly, plants with reduced expression of PDS3 exhibit reduced chlorophyll levels, up to complete albino and dwarfism.

Alcohol dehydrogenase (ADH1) comprises a group of dehydrogenase enzymes which catalyse the interconversion between alcohols and aldehydes or ketones with the concomitant reduction of NAD+ or NADP+. The principal metabolic purpose for this enzyme is the breakdown of alcoholic toxic substances within tissues. Plants harboring reduced ADH1 expression exhibit increase tolerance to allyl alcohol. Accordingly, plants with reduced ADH1 are resistant to the toxic effect of allyl alcohol, therefore their regeneration was carried out with allyl alcohol selection.

Two well-established miRNAs were chosen to be modified, miR-173 and miR-390, that were previously shown to be expressed throughout plant development [Zielezinski A et al., BMC Plant Biology (2015) 15: 144]. To introduce the modification, a 2-component system was used. First, the CRISPR/CAS9 system was used, to generate a cleavage in the miR-173 and miR-390 loci, through designed specific guide RNAs (Table 2, above), to promote homologous DNA repair (HDR) in the site. Second, A DONOR sequence, with the desired modification of the miRNA sequence, to target the newly assigned genes, was introduced as a template for the HDR (Table 2, above). In addition, since the secondary structure of the primary transcript of the miRNA (pri-miRNA) is important for the correct biogenesis and activity of the mature miRNA, further modifications were introduced in the complementary strand in the pri-miRNA and analysed in mFOLD (www(dot)unafold(dot)rna(dot)Albany(dot)edu) for structure conservation (data not shown). In total, two guides were designed for each miRNA loci, and two different DONOR sequences (modified miRNA sequences) were designed for each gene (Table 2, above).

### Example 10

### Bombardment and Plant Regeneration

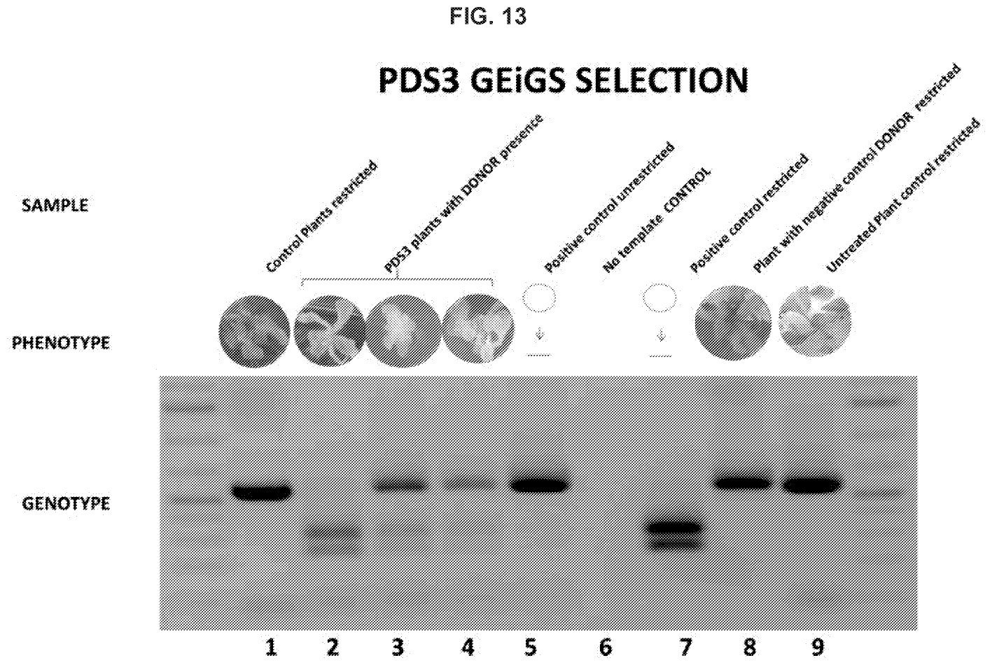

GEiGS constructs were bombarded into pre-prepared roots (as discussed in detail in the materials and experimental procedures section, above) and regenerated. Plantlets were selected via bleached phenotype for PDS3 transformants and survival on allyl alcohol treatment for ADH1 transformants. In order to validate Swap compared to no Swap, i.e. retained wild type, these plants were subsequently screened for insertion through specific primers spanning the modified region followed by restriction digest (FIG. 13).

### Example 11

### Genotype Validation of Phenotype Selection

As discussed above, the Proof of Concept (POC) for the gene editing system was established using well known phenotypic traits, Phytoene desaturase (PDS3) and Alcohol desaturase (ADH1) as targets.

As mentioned above, plants harboring reduced ADH1 expression exhibit increase tolerance to allyl alcohol. Therefore, bombarded plants for modified miRNA to target ADH1 were regenerated in media containing 30 mM allyl alcohol and compared to the regeneration rate of control plants. 118 GEiGS #3+SWAP11 allyl alcohol selected plants survived, compared to 51 control plants on allyl alcohol media (data not shown). Of the selected GEiGS #3+SWAP11, 5 were shown to harbour the DONOR (data not shown). The large amount of plants regenerating in the DONOR-treated plants, might be due to transient expression, during the bombardment process, as well.

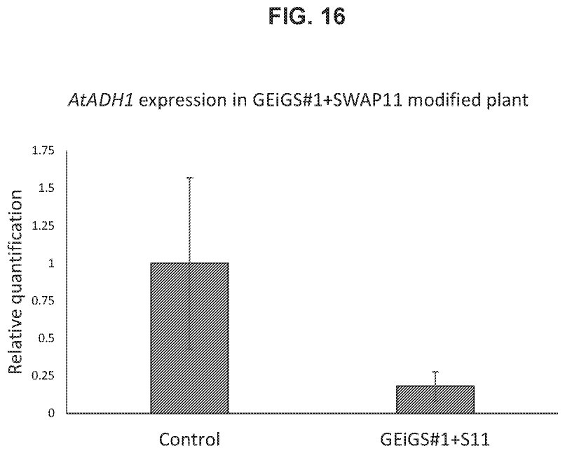

Thus, PDS3 and ADH1 selection through bleached phenotype (FIG. 16) and allyl alcohol selection (FIG. 17), respectively, give an ideal means for transformed plantlet selection for genotyping.

Swap region of 4 kb was assessed primarily through internal primers and specific amplicon differentiation of original wild type to insertion via restriction enzyme digestion variation.

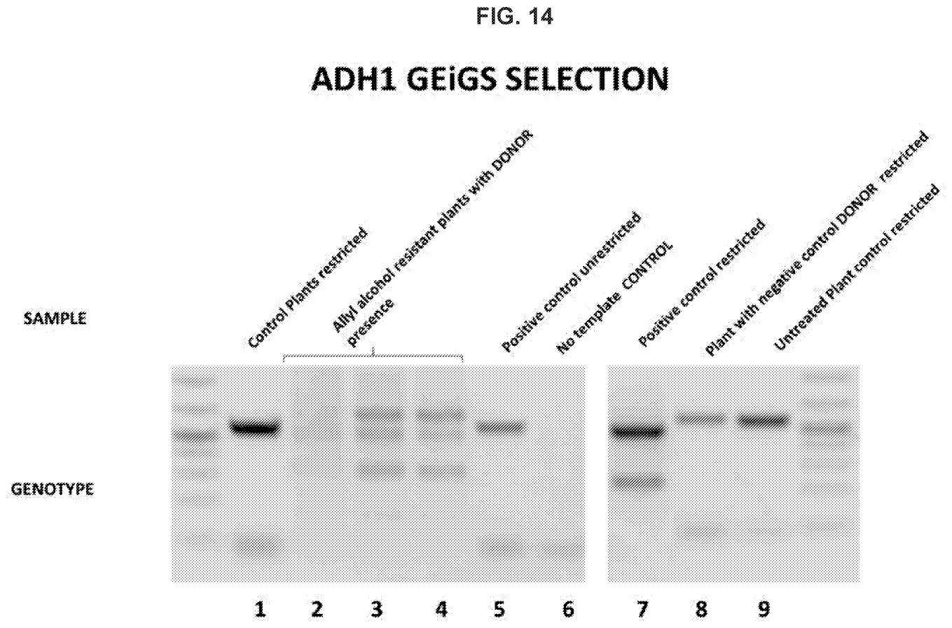

ADH1 (FIG. 14) showed a comparative genotype of allyl alcohol selected plants with the expected DONOR presence restriction pattern when compared to restricted and non-restricted DONOR plasmid. PDS3 (FIG. 13) showed a comparison of bombarded samples phenotypes with and without DONOR and their respective differential restriction enzyme digestion patterns compared to that of restricted and non-restricted DONOR plasmid. These results provided a clear association of PDS3 albino/bleached phenotype to the expected restriction pattern. Subsequent external PCR combining specific internal, within the Swap region, in conjunction with external primer, outside and specific to the genomic region to swap into was carried out (data not shown). Further validation of the Swap was obtained through Sanger sequencing of the PCR amplicons, in order to assess heterozygous, homozygous, or presence of DONOR Swap (data not shown).

### Example 12

### MODIFIED miRNA Reduce the Expression of their New Target Gene

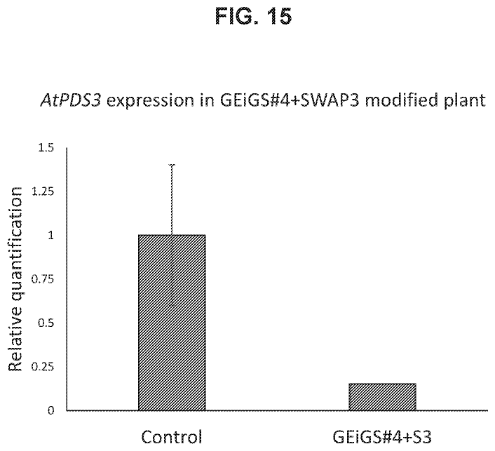

In order to verify the potential of the modified miRNAs in the GEiGS system to down regulate the expression of their newly designated targets, gene expression analysis was carried out using qRT-PCR (quantitative Real-Time PCR). RNA was extracted and reverse transcribed, from the positively identified regenerated plants and compared to regenerated plants, treated in parallel, but were not introduced with the relevant modifying constructs. In the case, where miR-173 was modified to target PDS3 (GEiGS #4+SWAP4), a reduction of 83% in the gene expression level, on average, was observed (FIG. 15). In plants with modified miR-390 to target ADH1 (GEiGS #3+SWAP11), a similar change in gene expression was observed, 82% of the levels in the control plants (FIG. 16). Taken together, these results substantiate the gene editing methods of modifying endogenous miRNAs to successfully target new genes and reduce their expression, by replacing the target recognition sequence in the miRNA transcript in the endogenous locus.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

What is claimed is:

1. A method of modifying a gene encoding or processed into a non-coding RNA molecule having no RNA silencing activity in a eukaryotic cell, wherein the gene encoding or processed into the non-coding RNA molecule is positioned in a coding gene, the method comprising introducing into the eukaryotic cell a DNA editing agent conferring a silencing specificity of said non-coding RNA molecule towards a target RNA of interest, thereby modifying the gene encoding or processed into the non-coding RNA molecule.

2. A method of modifying a gene encoding or processed into a RNA silencing molecule to a target RNA in a eukaryotic cell, wherein the gene encoding or processed into the non-coding RNA molecule is positioned in a coding gene, the method comprising introducing into the eukaryotic cell a DNA editing agent which redirects a silencing specificity of said RNA silencing molecule towards a second target RNA, said target RNA and said second target RNA being distinct, thereby modifying the gene encoding or processed into the RNA silencing molecule.

3. A method of modifying a gene encoding or processed into a non-coding RNA molecule having no RNA silencing activity in a eukaryotic cell, the method comprising introducing into the eukaryotic cell a DNA or RNA editing agent conferring a silencing specificity of said non-coding RNA molecule towards a target RNA of interest, wherein said DNA or RNA editing agent elicits base editing, thereby modifying the gene encoding or processed into the non-coding RNA molecule.

4. A method of modifying a gene encoding or processed into a RNA silencing molecule to a target RNA in a eukaryotic cell, the method comprising introducing into the eukaryotic cell a DNA or RNA editing agent which redirects a silencing specificity of said RNA silencing molecule towards a second target RNA, said target RNA and said second target RNA being distinct, and wherein said DNA or RNA editing agent elicits base editing, thereby modifying the gene encoding or processed into the RNA silencing molecule.

5. A method of modifying a gene encoding or processed into a non-coding RNA molecule having no RNA silencing activity in a eukaryotic cell, the method comprising introducing into the eukaryotic cell a DNA editing agent conferring a silencing specificity of said non-coding RNA molecule towards a target RNA of interest, wherein said target RNA of interest is a transcript of a gene selected from the group consisting of a housekeeping gene, a dominant gene, a gene comprising a high copy number, and a gene associated with cell apoptosis, thereby modifying the gene encoding or processed into the non-coding RNA molecule.

6. A method of modifying a gene encoding or processed into a RNA silencing molecule to a target RNA in a eukaryotic cell, the method comprising introducing into the eukaryotic cell a DNA editing agent which redirects a silencing specificity of said RNA silencing molecule towards a second target RNA, wherein said second target RNA is a transcript of a gene selected from the group consisting of a housekeeping gene, a dominant gene, a gene comprising a high copy number, and a gene associated with cell apoptosis, said target RNA and said second target RNA being distinct, thereby modifying the gene encoding or processed into the RNA silencing molecule.

7. The method of any one of claims 3-6, wherein the gene encoding or processed into the non-coding RNA molecule or into the RNA silencing molecule is positioned in a non-coding gene.

8. The method of any one of claims 3-6, wherein the gene encoding or processed into the non-coding RNA molecule or into the RNA silencing molecule is positioned in a coding gene.

9. The method of any one of claim 1, 2 or 8, wherein the gene encoding or processed into the non-coding RNA molecule or into the RNA silencing molecule is positioned within an exon of coding gene.

10. The method of any one of claim 1, 2, 8 or 9, wherein the gene encoding or processed into the non-coding RNA molecule or into the RNA silencing molecule is positioned within an exon encoding an untranslated region (UTR) of a coding gene.

11. The method of any one of claim 1, 2, 8, 9 or 10, wherein the gene encoding or processed into the non-coding RNA molecule or into the RNA silencing molecule is positioned within an intron of coding gene.

12. The method of any one of claims 1-11, wherein the gene encoding or processed into the non-coding RNA molecule or into the RNA silencing molecule is endogenous to the eukaryotic cell.

13. The method of any one of claim 1, 3, 5 or 7-12, wherein said modifying said gene encoding or processed into said non-coding RNA molecule comprises imparting said non-coding RNA molecule with at least 45% complementarity towards said target RNA of interest.

14. The method of any one of claim 2, 4, 6 or 7-12, wherein said modifying said gene encoding or processed into the RNA silencing molecule comprises imparting said RNA silencing molecule with at least 45% complementarity towards said second target RNA.

15. The method of any one of claim 1, 3, 5 or 7-13, wherein said silencing specificity of said non-coding RNA molecule is determined by measuring an RNA or protein level of said target RNA of interest.

16. The method of any one of claim 2, 4, 6, 7-12 or 14, wherein said silencing specificity of said RNA silencing molecule is determined by measuring an RNA or protein level of said second target RNA.

17. The method of any one of claims 1-16, wherein said silencing specificity of the non-coding RNA molecule or the RNA silencing molecule is determined phenotypically.

18. The method of any one of claims 1-17, wherein said silencing specificity of the non-coding RNA molecule or the RNA silencing molecule is determined genotypically.

19. The method of any one of claims 1-18, wherein said non-coding RNA molecule or said RNA silencing molecule is processed from a precursor.

20. The method of any one of claims 1-19, wherein said non-coding RNA molecule or said RNA silencing molecule is processed into small RNA engaged with RNA-induced silencing complex (RISC).

21. The method of claim 20, wherein said small RNA engaged with said RISC is selected from the group consisting of a small interfering RNA (siRNA), a short hairpin RNA (shRNA), a microRNA (miRNA), a Piwi-interacting RNA (piRNA), phased small interfering RNA (phasiRNA), trans-acting siRNA (tasiRNA), a small nuclear RNA (snRNA), a small nucleolar RNA (snoRNA), a long non-coding RNA (lncRNA), a ribosomal RNA (rRNA), transfer RNA (tRNA), a repeat-derived RNA, and an autonomous and non-autonomous transposable element RNA.

22. The method of claim 20 or 21, wherein said small RNA engaged with said RISC is modified to preserve originality of structure and to be recognized by cellular RNAi factors.

23. The method of any one of claims 1-22, wherein said modifying said gene is affected by a modification selected from the group consisting of a deletion, an insertion, a point mutation and a combination thereof.

24. The method of claim 23, wherein said modification is in:
a stem region of said non-coding RNA molecule or said RNA silencing molecule; or
a loop region of said non-coding RNA molecule or said RNA silencing molecule; or
a non-structured region of said non-coding RNA molecule or said RNA silencing molecule; or
a stem region and a loop region of said non-coding RNA molecule or said RNA silencing molecule; or
a stem region and a loop region and in non-structured region of said non-coding RNA molecule or said RNA silencing molecule.

25. The method of any one of claims 23-24, wherein said modification comprises a modification of at most 200 nucleotides.

26. The method of any one of claims 23-25, wherein said method does not comprise introducing into said eukaryotic cell donor oligonucleotides.

27. The method of any one of claims 23-25, wherein said method further comprises introducing into said eukaryotic cell donor oligonucleotides.

28. The method of any one of claims 1-27, wherein said DNA editing agent comprises at least one sgRNA.

29. The method of any one of claim 1-2 or 5-28, wherein said DNA editing agent elicits base editing.

30. The method of any one of claims 1-29, wherein said DNA or RNA editing agent does not comprise an endonuclease.

31. The method of any one of claims 1-29, wherein said DNA or RNA editing agent comprises an endonuclease.

32. The method of claim 31, wherein said endonuclease comprises Cas9.

33. The method of claim 31 or 32, wherein said endonuclease comprises a catalytically inactive endonuclease.

34. The method of any one of claim 3-4 or 29-33, wherein said DNA or RNA editing agent comprises an enzyme which is capable of epigenetic editing.

35. The method of claim 34, wherein said enzyme which is capable of said epigenetic editing is selected from the group consisting of a DNA methyltransferase, a methylase, an acetyltransferase; optionally wherein wherein said enzyme which is capable of said epigenetic editing is selected from the group consisting of a DNA (cytosine-5)-methyltransferase 3A (DNMT3a), a Histone acetyltransferase p300, a Ten-eleven translocation methylcytosine dioxygenase 1 (TET1), Lysine (K)-specific demethylase 1A (LSD1) and Calcium and integrin binding protein 1 (CIB1).

36. The method of any one of claims 1-35, wherein said DNA editing agent comprises a DNA editing system selected from the group consisting of a meganuclease, a zinc finger nucleases (ZFN), a transcription-activator like effector nuclease (TALEN), CRISPR-endonuclease, dCRISPR-endonuclease, and a homing endonuclease.

37. The method of any one of claims 1-36, wherein said DNA editing agent is applied to the cell as DNA, RNA or RNP.

38. The method of any one of claims 1-37, wherein said DNA or RNA editing agent is linked to a reporter for monitoring expression in a eukaryotic cell.

39. The method of any one of claims 1-38, wherein said target RNA of interest or said second target RNA is endogenous to said eukaryotic cell.

40. The method of any one of claims 1-38, wherein said target RNA of interest or said second target RNA is exogenous to said eukaryotic cell.

41. The method of any one of claim 1-4 or 7-40, wherein said target RNA of interest or said second target RNA is a transcript of a gene selected from the group consisting of a housekeeping gene, a dominant gene, a gene comprising a high copy number and a gene associated with cell apoptosis.

42. The method of any one of claim 5-6 or 41, wherein said gene associated with cell apoptosis is selected from the group consisting of BAX, PUMA and NOXA.

43. The method of any one of claims 1-42, wherein said eukaryotic cell is obtained from a eukaryotic organism selected from the group consisting of a plant, a mammal, an invertebrate, an insect, a nematode, a bird, a reptile, a fish, a crustacean, a fungi and an algae.

44. A plant cell generated according to the method of any one of claims 1-43.

45. A plant comprising the plant cell of claim 44.

46. A method of producing a plant comprising a reduced expression of a housekeeping gene, a dominant gene, a gene comprising a high copy number and/or a gene associated with cell apoptosis, the method comprising:
(a) breeding the plant of claim 45; and
(b) selecting for progeny plants that have reduced expression of said housekeeping gene, said dominant gene, said gene comprising a high copy number, and/or gene associated with cell apoptosis, and which do not comprise said DNA editing agent,
thereby producing said plant with reduced expression of said housekeeping gene, said dominant gene, said gene comprising a high copy number and/or gene associated with cell apoptosis.

47. The method of claim 46, wherein said breeding comprises crossing or selfing.

48. A method producing a plant or plant cell of any one of claims 44-45 comprising growing the plant or plant cell under conditions which allow propagation.

49. A seed of the plant of claim 45, or the plant generated according to the method of any one of claims 46-48.

50. A method of treating a disease in a subject in need thereof, the method comprising modifying a gene encoding or processed into a non-coding RNA molecule or into an RNA silencing molecule according to the method of any one of claims 1-43, wherein said target RNA of interest or said second target RNA is a transcript of a housekeeping gene, a dominant gene, a gene comprising a high copy number, and/or a gene associated with cell apoptosis, associated with an onset or progression of the disease.
